(19) United States
(12) Patent Application Publication
(10) Pub. No.: US 2024/0147746 A1
(43) Pub. Date: May 2, 2024

(54) ORGANIC COMPOUND AND USE THEREOF

(71) Applicants: Wuhan Tianma Microelectronics Co., Ltd., Wuhan (CN); Wuhan Tianma Microelectronics Co., Ltd. Shanghai Branch, Shanghai (CN)

(72) Inventors: Dongyang Deng, Wuhan (CN); Wenpeng Dai, Wuhan (CN); Lilian Kuang, Wuhan (CN)

(73) Assignees: Wuhan Tianma Microelectronics Co., Ltd., Wuhan (CN); Wuhan Tianma Microelectronics Co., Ltd. Shanghai Branch, Shanghai (CN)

(21) Appl. No.: 18/393,814

(22) Filed: Dec. 22, 2023

(30) Foreign Application Priority Data
Apr. 4, 2023 (CN) 202310377368.9

(51) Int. Cl. H10K 50/11; C07D 471/04; C07F 7/08; H10K 50/15; H10K 50/16; H10K 50/17; H10K 85/60

(52) U.S. Cl. CPC H10K 50/11; C07D 471/04; C07F 7/0816; H10K 50/15; H10K 50/16; H10K 50/171; H10K 85/6572

(57) ABSTRACT

Provided are an organic compound and use thereof, where the organic compound has excellent thermal stability and thin-film stability, is stable when the organic electroluminescent device works, and when applied to the organic electroluminescent device as a charge generation layer material, enables the device to have a high current efficiency, a low drive voltage and a long lifetime.

CROSS-REFERENCE TO RELATED APPLICATION(S)

This application claims priority to Chinese Patent Application No. CN 202310377368.9 filed Apr. 4, 2023, the disclosure of which is incorporated herein by reference in its entirety.

### TECHNICAL FIELD

The present disclosure belongs to the technical field of organic light-emitting materials and relates to an organic compound and use thereof.

### BACKGROUND

Light-emitting devices are devices for converting electric energy into light energy by using organic compounds. Organic light-emitting devices may be formed in a variety of structures, one of which is a tandem organic light-emitting device. In the tandem device, light-emitting units each including a light-emitting layer are stacked between the anode and the cathode. A charge generation layer is disposed between adjacent light-emitting portions for the generation and movement of charges, and the charge generation layer requires a low drive voltage and a high efficiency.

At present, the charge generation layer in the tandem device faces low material selectivity and also has problems such as high drive voltages and low efficiencies mainly due to deficiencies of the materials themselves and poor matching of energy levels of adjacent layers. Therefore, it is desirable in the art to be able to develop materials that can be used in the charge generation layer to improve the efficiency and lifetime of the OLED (organic light-emitting diode) device and reduce the drive voltage.

### SUMMARY

The present disclosure is to provide an organic compound and use thereof.

A first aspect of the present disclosure is to provide an organic compound. The organic compound has a structure represented by Formula I:

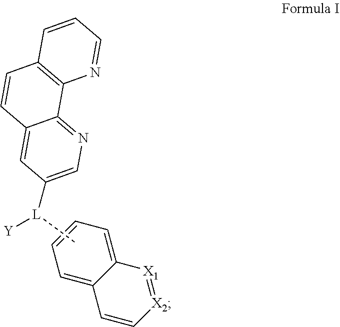

wherein X₁ and X₂ are each independently selected from CR₁ or N; R₁ is selected from hydrogen, a deuterium atom, substituted or unsubstituted C1 to C20 alkyl, substituted or unsubstituted C6 to C30 aryl, substituted or unsubstituted C3 to C20 cycloalkyl, substituted or unsubstituted C1 to C20 alkoxy, substituted or unsubstituted C3 to C30 heterocyclyl or a substituted or unsubstituted C6 to C30 fused ring group;

L is selected from substituted or unsubstituted C6 to C30 aryl or substituted or unsubstituted C3 to C30 heteroaryl; and

Y is selected from substituted or unsubstituted C6 to C30 aryl or substituted or unsubstituted C3 to C30 heteroaryl.

In the present disclosure, C6 to C30 may each independently be C6, C9, C10, C12, C13, C14, C15, C16, C18, C20, C22, C24, C26, C28 or C29, etc.

C3 to C30 may each independently be C4, C6, C9, C10, C12, C13, C14, C15, C16, C18, C20, C22, C24, C26, C28 or C29, etc.

C1 to C20 may each independently be C1, C3, C5, C6, C9, C10, C12, C13, C14, C15, C16, C18 or C20, etc.

C3 to C20 may each independently be C3, C5, C6, C9, C10, C12, C13, C14,

C15, C16, C18 or C20, etc.

The compound provided by the present disclosure diverges in three directions with the L group as the center, and the distribution of HOMO and LUMO energy levels, the triplet energy level and the singlet energy level are adjusted through the selection of the L and Y groups so that the compound has suitably separated HOMO and LUMO and can better match the adjacent layers. The compound is twisted, has strong spatially steric nature and thus has high singlet and triplet energy levels and a high glass transition temperature so that the compound has excellent thermal stability and thin-film stability.

In addition, such a material is simple to prepare, and the raw materials thereof are easily available. When the material is applied to an OLED device as an NCGL material, the luminescence efficiency of the device can be significantly improved, and the drive voltage of the device can be effectively reduced. Therefore, the material is an OLED material with excellent performance.

A second aspect of the present disclosure is to provide a charge generation layer material. The charge generation layer material includes the organic compound described in the first aspect.

A third aspect of the present disclosure is to provide an electron transport layer material. The electron transport layer material includes the organic compound described in the first aspect.

A fourth aspect of the present disclosure is to provide an OLED device. The OLED device includes an anode, a cathode, at least two organic light-emitting units disposed between the anode and cathode, and a charge generation layer connected to adjacent light-emitting units, wherein the charge generation layer includes the organic compound described in the first aspect.

A fifth aspect of the present disclosure is to provide an OLED device. The OLED device includes an anode, a cathode, at least two organic light-emitting units disposed between the anode and cathode, and a charge generation layer connected to adjacent light-emitting units, wherein each of the at least two organic light-emitting units includes the organic compound described in the first aspect.

A sixth aspect of the present disclosure is to provide a display panel. The display panel includes the OLED device described in the fourth or fifth aspect.

A seventh aspect of the present disclosure is to provide an organic light-emitting display device. The organic light-emitting display device includes the display panel described in the sixth aspect.

An eighth aspect of the present disclosure is to provide an electronic device. The electronic device includes the display panel described in the seventh aspect.

Compared with the prior art, the present disclosure has the beneficial effects described below.

The organic compound provided by the present disclosure has excellent thermal stability and thin-film stability, is stable when the organic electroluminescent device works, and when applied to the organic electroluminescent device as a charge generation layer material, enables the device to have a high current efficiency, a low drive voltage and a long lifetime.

### BRIEF DESCRIPTION OF DRAWINGS

FIG. 1 is a schematic structural diagram of a tandem OLED device according to the present disclosure; in the figure, 1 is a substrate, 2 is an anode, 3 is a first hole transport layer, 4 is a second hole transport layer, 5 is a first light-emitting layer, 6 is a first electron transport layer, 7 is an N-type electron generation layer, 8 is a P-type electron generation layer, 9 is a third hole transport layer, 10 is a second light-emitting layer, 11 is a second electron transport layer, 12 is a third electron transport layer, 13 is a cathode, and 14 is a capping layer.

### DETAILED DESCRIPTION

Technical solutions of the present disclosure are further described below through embodiments. It is to be understood by those skilled in the art that the embodiments described below are used for a better understanding of the present disclosure and are not to be construed as specific limitations to the present disclosure.

A first aspect of the present disclosure is to provide an organic compound, wherein the organic compound has a structure represented by Formula I:

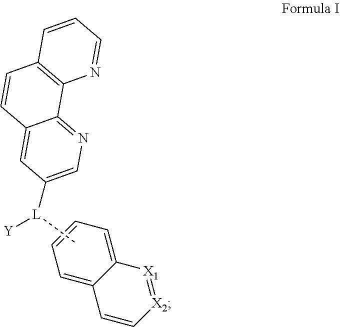

wherein X₁ and X₂ are each independently selected from CR₁ or N; R₁ is selected from hydrogen, a deuterium atom, substituted or unsubstituted C1 to C20 alkyl, substituted or unsubstituted C6 to C30 aryl, substituted or unsubstituted C3 to C20 cycloalkyl, substituted or unsubstituted C1 to C20 alkoxy, substituted or unsubstituted C3 to C30 heterocyclyl or a substituted or unsubstituted C6 to C30 fused ring group;

L is selected from substituted or unsubstituted C6 to C30 aryl or substituted or unsubstituted C3 to C30 heteroaryl; and

Y is selected from substituted or unsubstituted C6 to C30 aryl or substituted or unsubstituted C3 to C30 heteroaryl.

In the present disclosure, C6 to C30 may each independently be C6, C9, C10, C12, C13, C14, C15, C16, C18, C20, C22, C24, C26, C28 or C29, etc.

C3 to C30 may each independently be C4, C6, C9, C10, C12, C13, C14, C15, C16, C18, C20, C22, C24, C26, C28 or C29, etc.

C1 to C20 may each independently be C1, C3, C5, C6, C9, C10, C12, C13, C14, C15, C16, C18 or C20, etc.

C3 to C20 may each independently be C3, C5, C6, C9, C10, C12, C13, C14, C15, C16, C18 or C20, etc.

The compound provided by the present disclosure diverges in three directions with the L group as the center, and the distribution of HOMO and LUMO energy levels, the triplet energy level and the singlet energy level are adjusted through the selection of the L and Y groups so that the compound has suitably separated HOMO and LUMO and can better match the adjacent layers. The compound is twisted, has strong spatially steric nature and thus has high singlet and triplet energy levels and a high glass transition temperature so that the compound has excellent thermal stability and thin-film stability.

In addition, such a material is simple to prepare, and the raw materials thereof are easily available. When the material is applied to an OLED device as an NCGL material, the luminescence efficiency of the device can be significantly improved, and the drive voltage of the device can be effectively reduced. Therefore, the material is an OLED material with excellent performance.

In some preferred embodiments, a substituent in substituted C6 to C30 aryl or substituted C3 to C30 heteroaryl is selected from at least one of deuterium, cyano, halogen, unsubstituted or halogenated C1 to C10 linear or branched alkyl, unsubstituted or halogenated C1 to C10 alkoxy, C6 to C20 aryl, C2 to C20 heteroaryl or C6 to C18 arylamine.

In some preferred embodiments, one of X₁ and X₂ is selected from CR₁ and the other is selected from N.

In some preferred embodiments, X₁ and X₂ are both selected from N.

In some preferred embodiments, L is selected from phenyl, biphenyl, naphthyl, pyridyl,

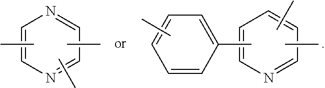

In the present disclosure, the compound formed by connecting phenanthroline and an N-substituted naphthalene group by an L group through the meta-position connection mode has suitable steric hindrance, and the three-dimensional configuration is suitable for the interaction between the compound and metals such as Yb, thereby enabling charge generation, effectively reducing the voltage of the device and improving the working stability of the device. For the compound formed by connecting phenanthroline and an N-substituted naphthyl group through the para-position connection mode, the electron cloud distribution and the spatial structure of the compound can be adjusted through the selection of the Y group, thereby enabling the charge generation and achieving a low voltage.

In some preferred embodiments, L is selected from

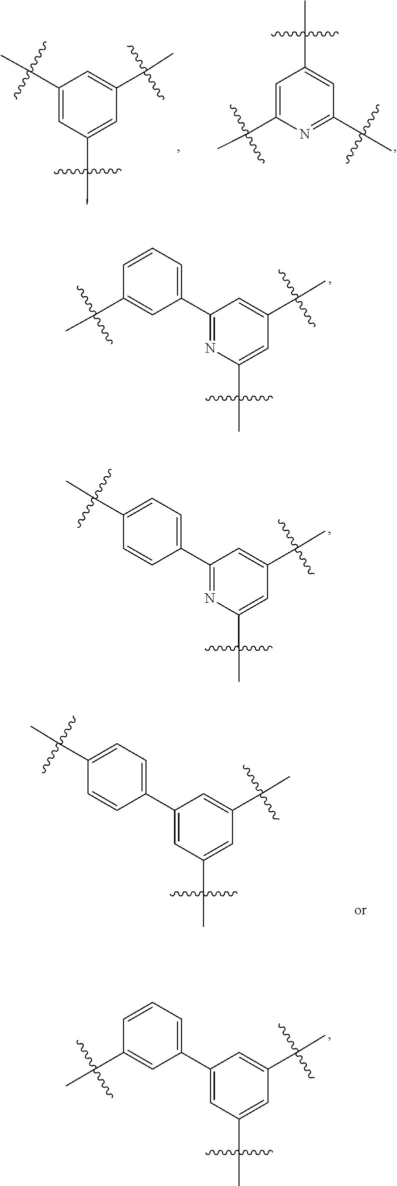 [SMILES: *c1cc(*)cc(*)c1 ; *c1cc(*)nc(*)c1 ; *c1cccc(-c2cc(*)cc(*)n2)c1 ; *c1ccc(-c2cc(*)cc(*)n2)cc1 ; *c1ccc(-c2cc(*)cc(*)c2)cc1 ; *c1cccc(-c2cc(*)cc(*)c2)c1]

wherein the wavy line represents a position of attachment to a group.

In some preferred embodiments, Y is selected from substituted or unsubstituted phenyl, substituted or unsubstituted naphthyl, substituted or unsubstituted dibenzofuryl, substituted or unsubstituted fluorenyl, spirobifluorenyl, benzodibenzofuryl or substituted or unsubstituted dibenzothienyl.

In some preferred embodiments, Y is selected from any one of phenyl, biphenyl, naphthyl, anthryl, anthryl-phenyl, phenanthryl, phenanthryl-phenyl, pyrenyl, cyano, carbazolyl, acridinyl, phenanthrolinyl, triarylamino, dibenzothienyl, dibenzofuryl, benzoxazolyl, dimethylfluorenyl, spirobifluorenyl or benzoquinolyl.

In some preferred embodiments, the organic compound includes any one of the following compounds:

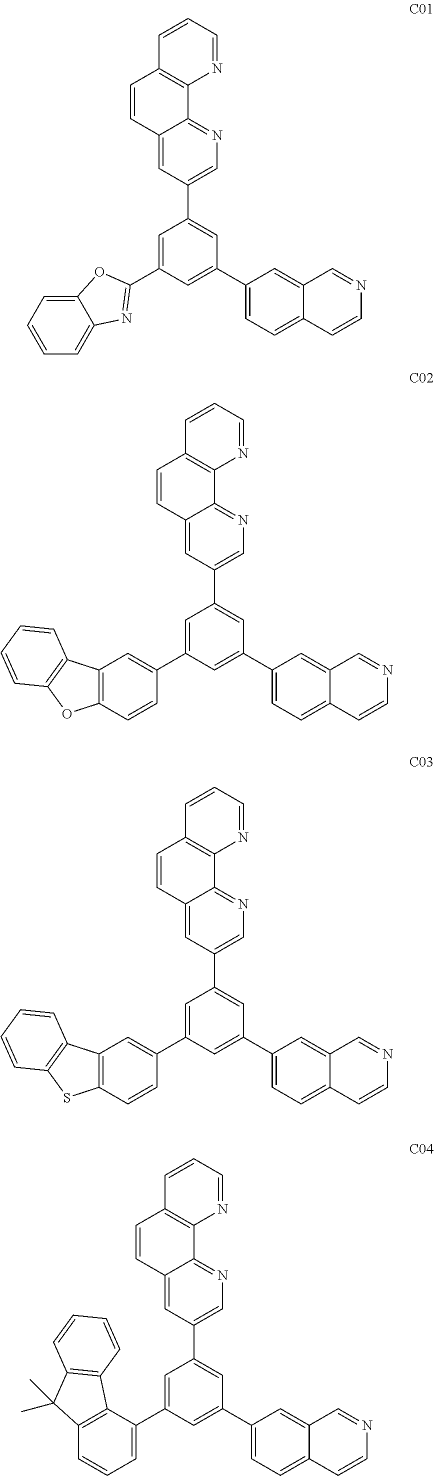

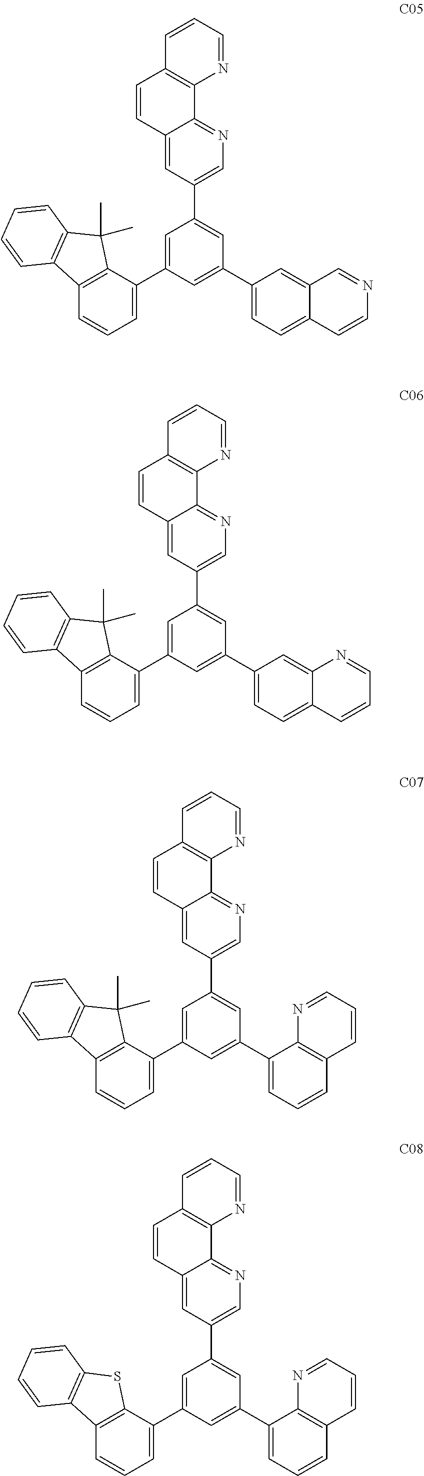

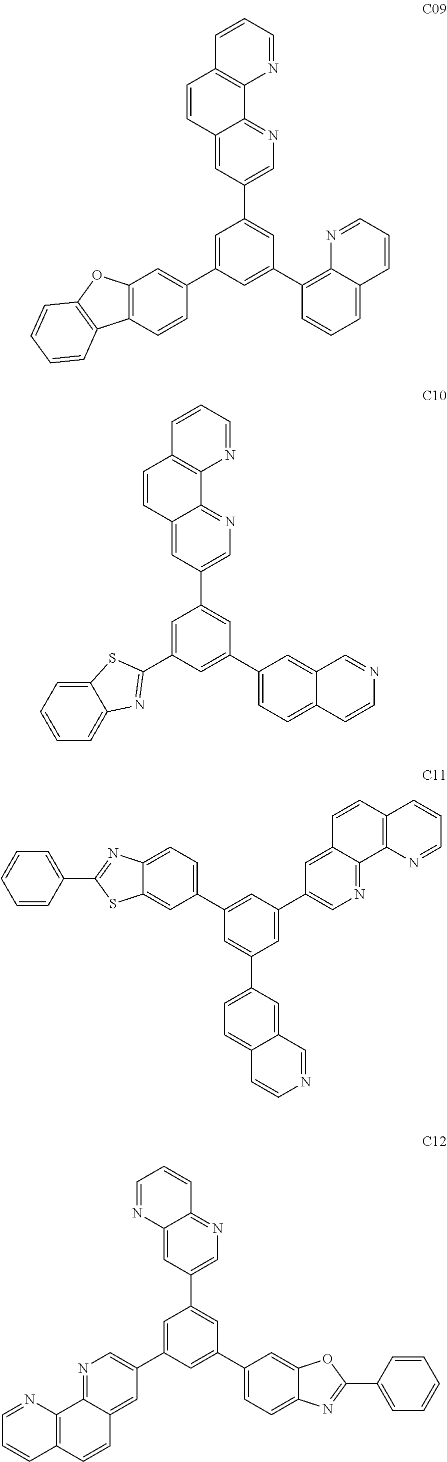

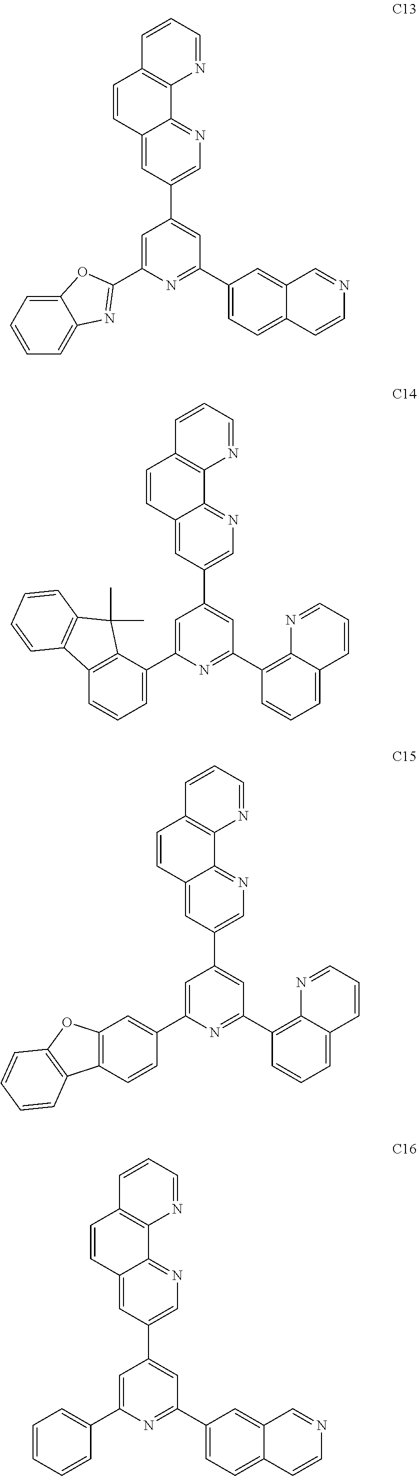

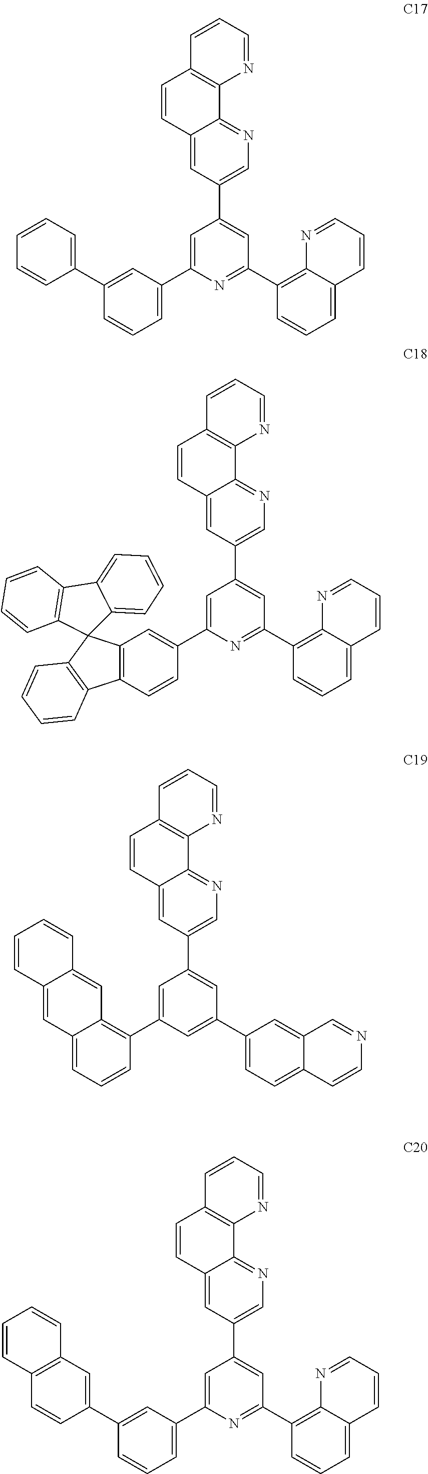

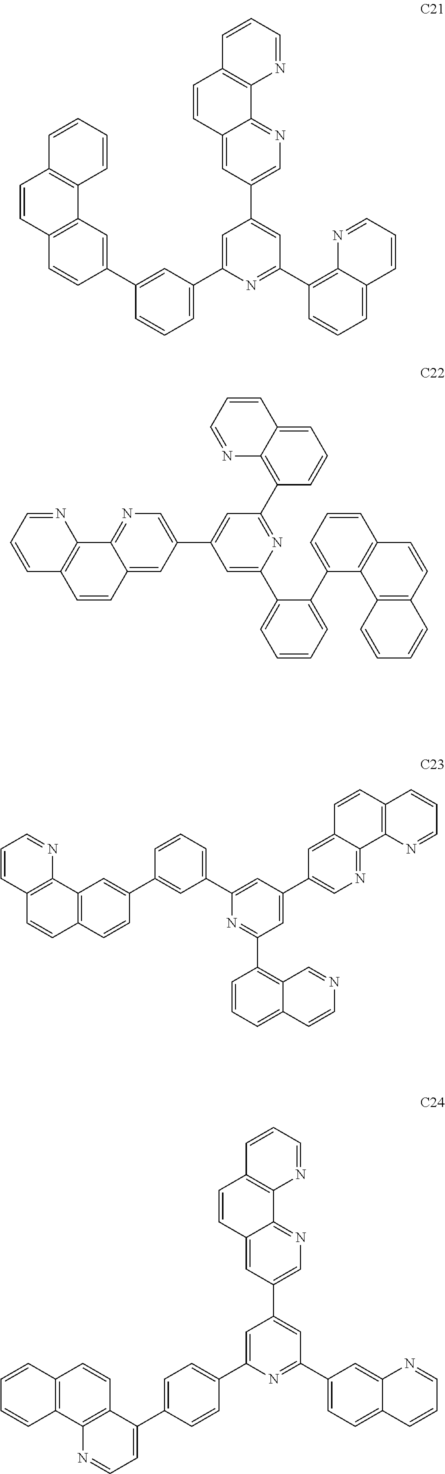

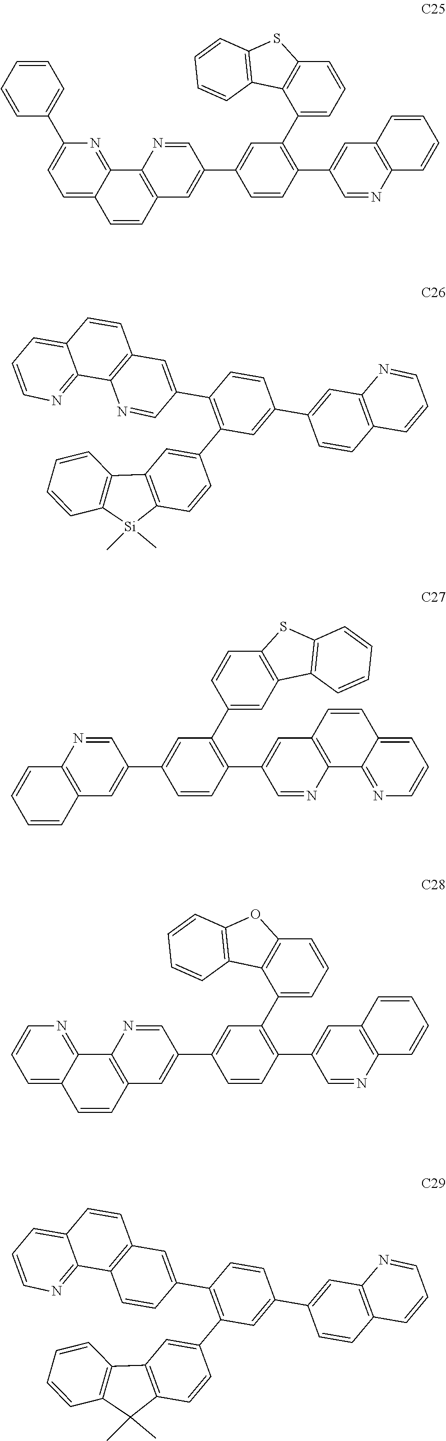

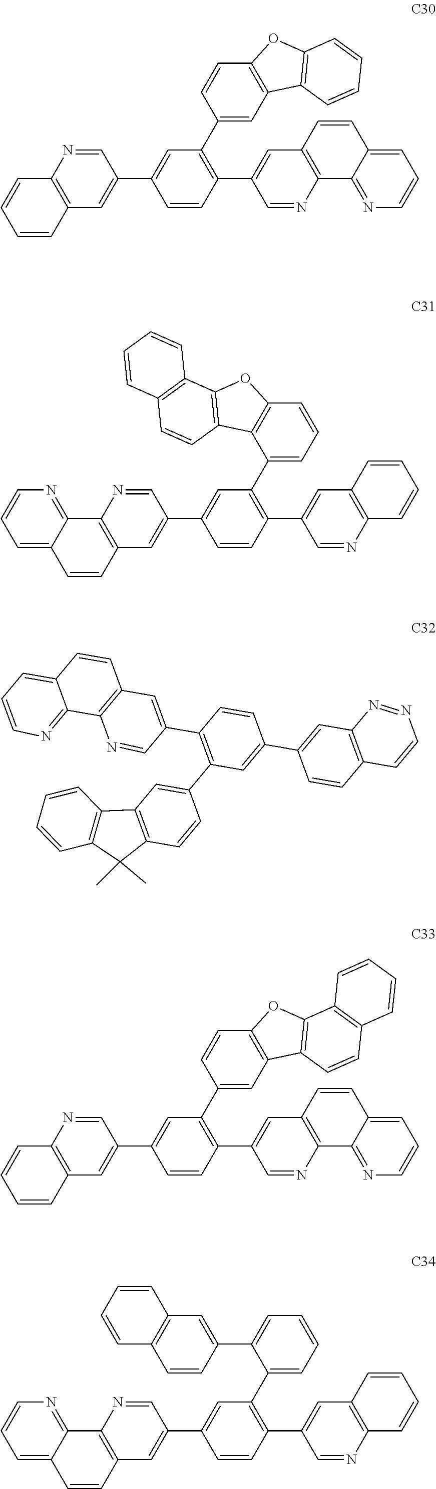

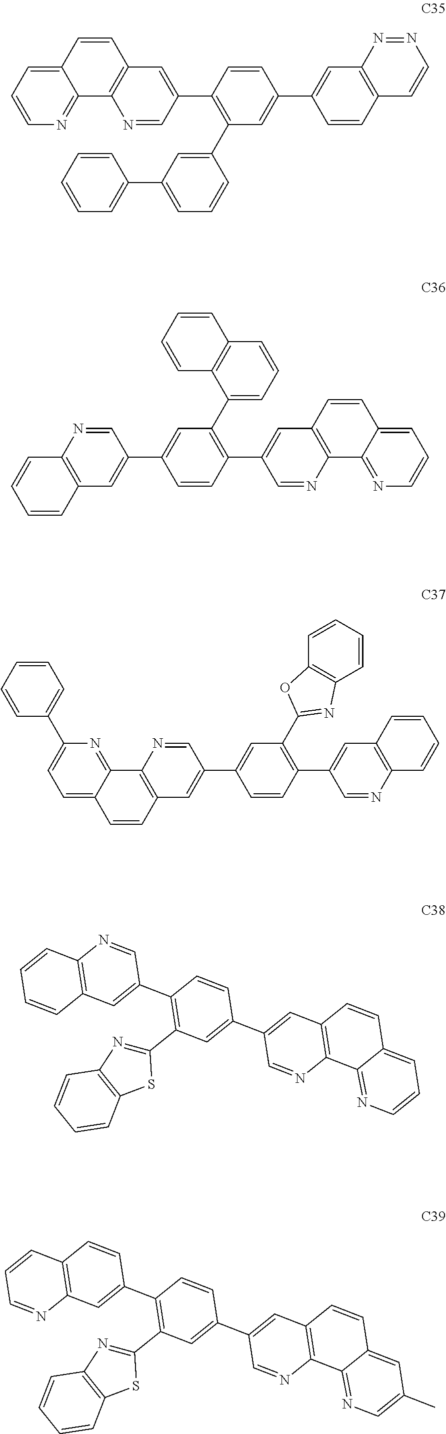

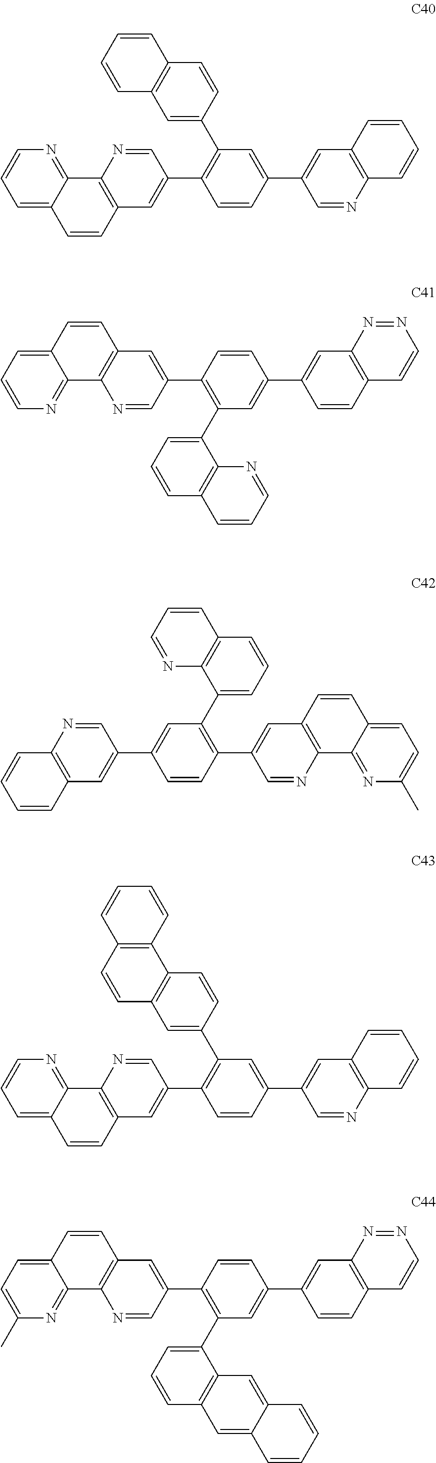

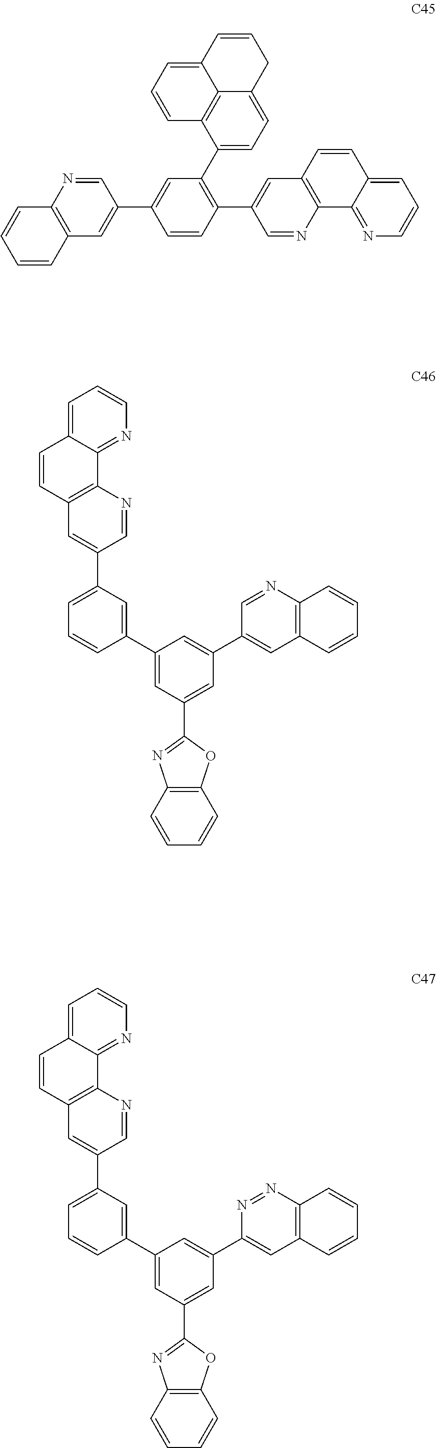

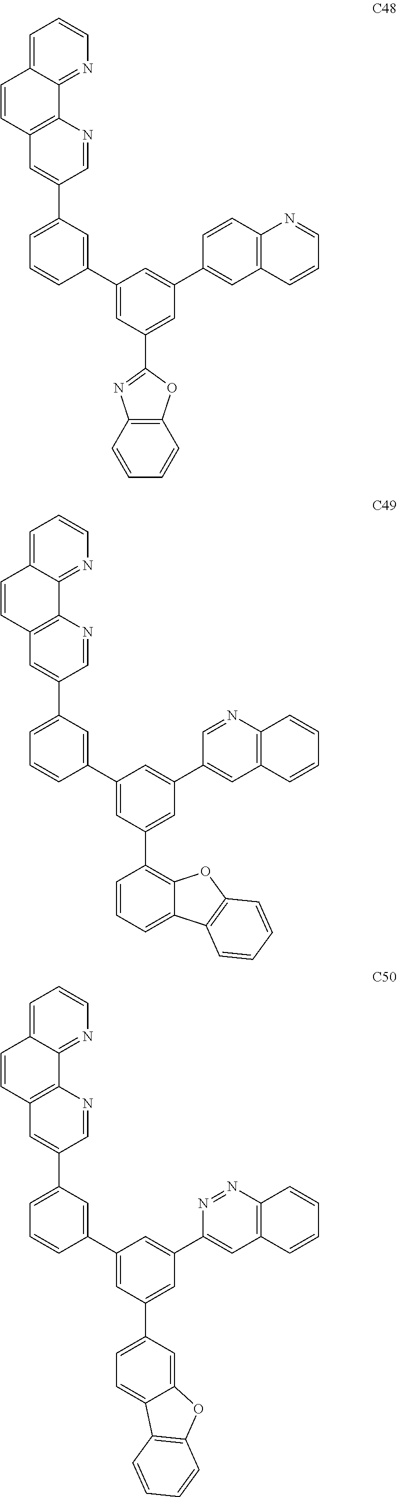

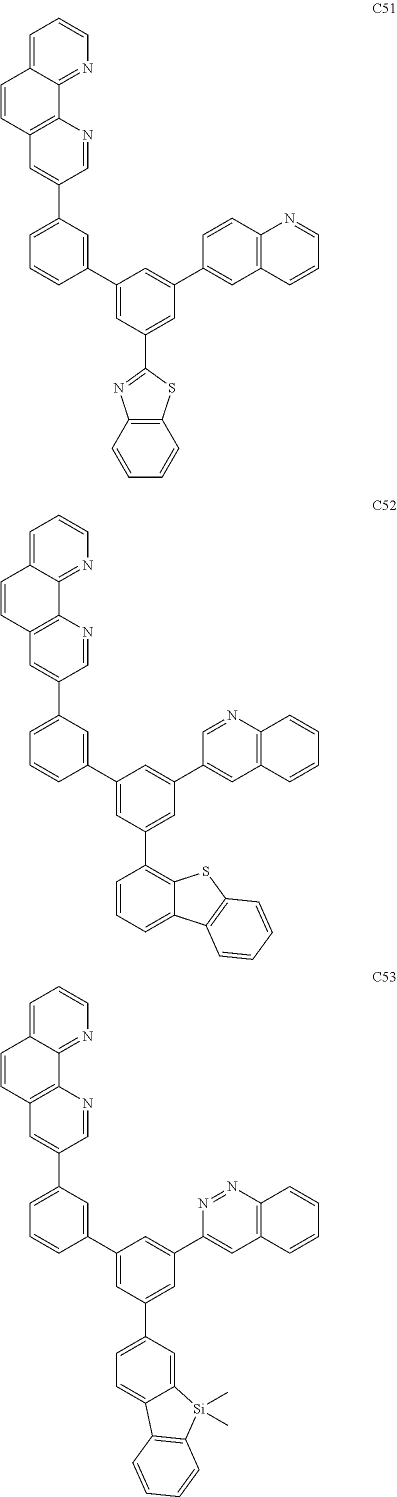

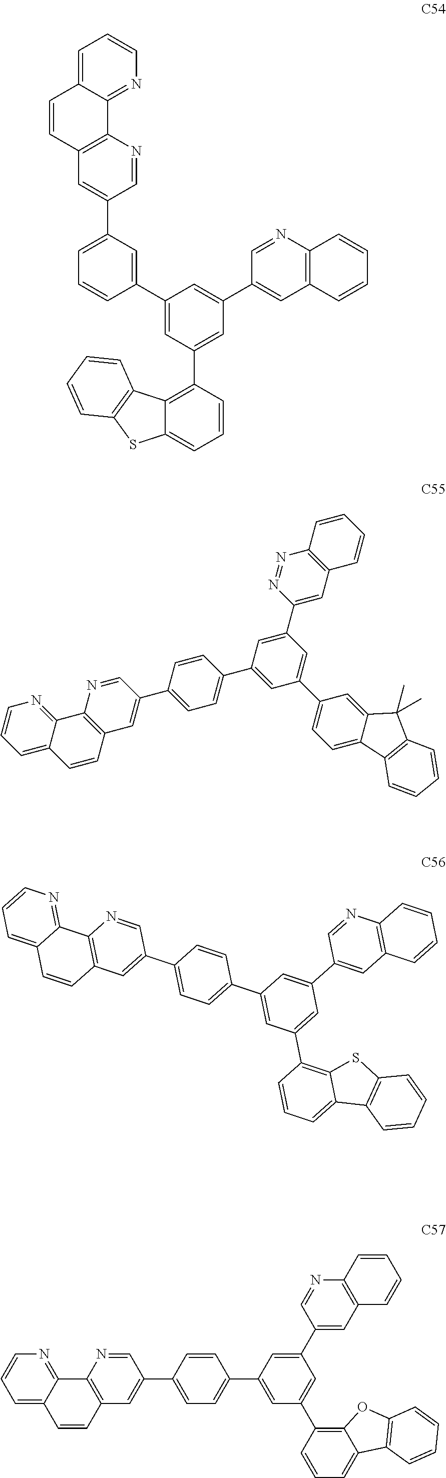

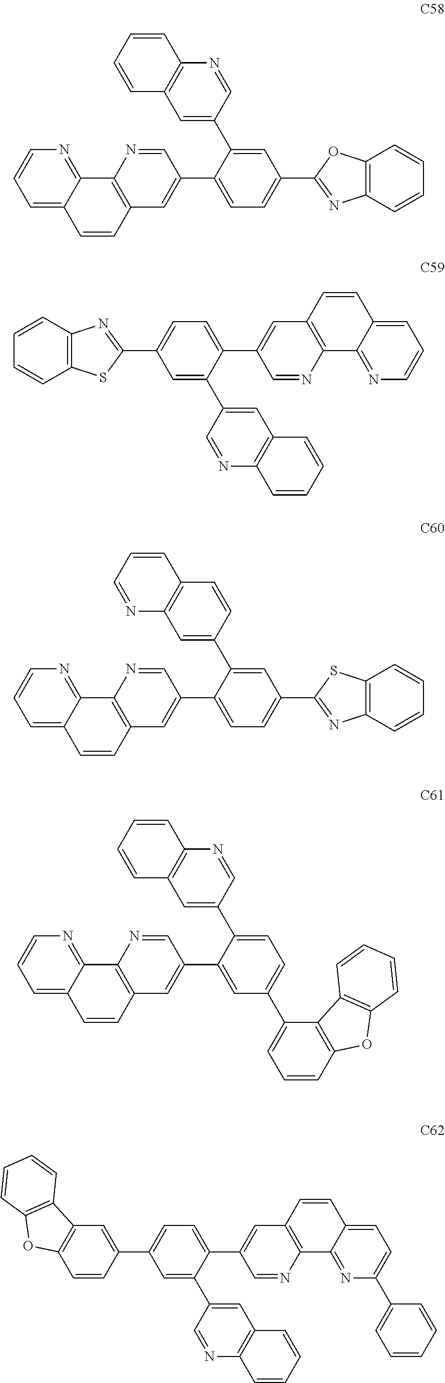

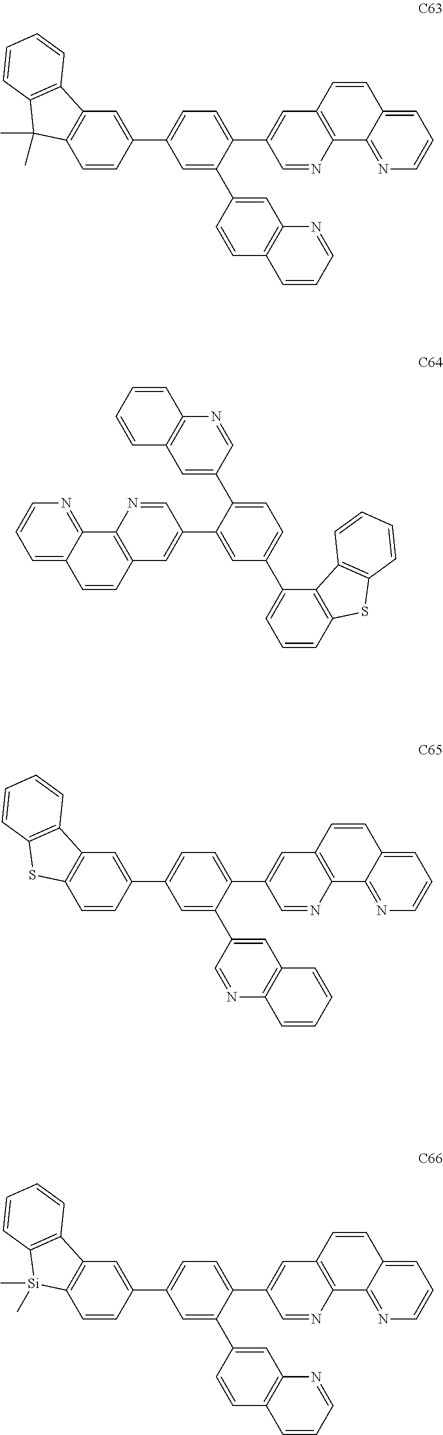

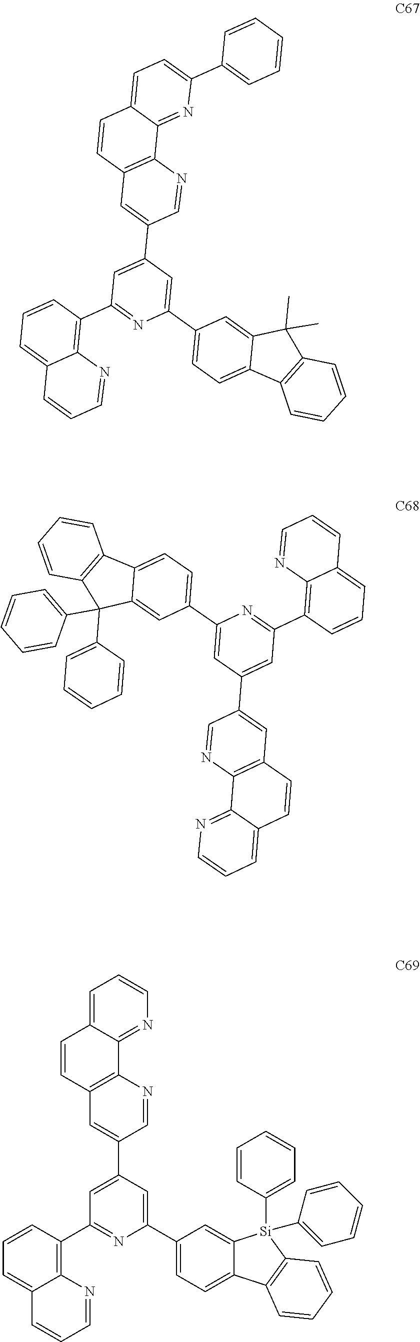

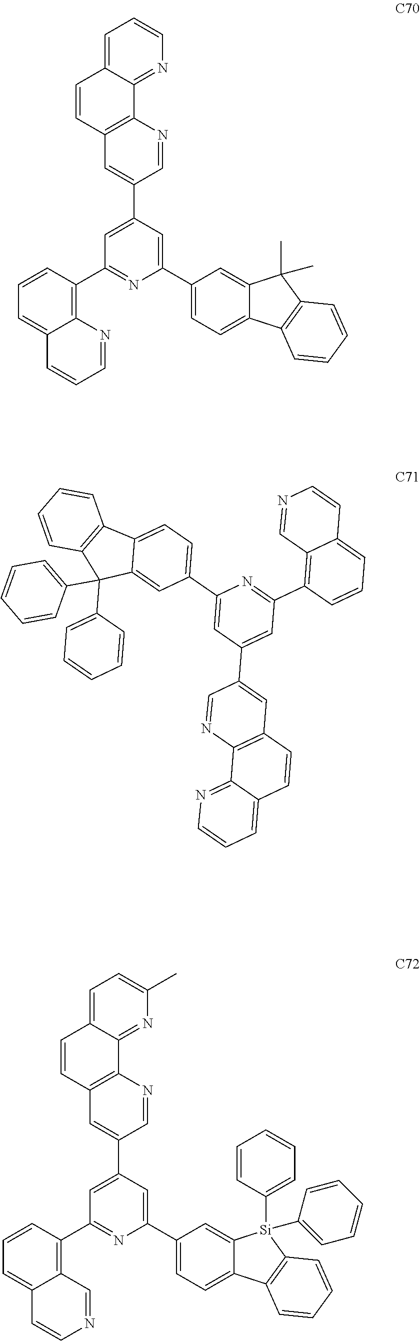

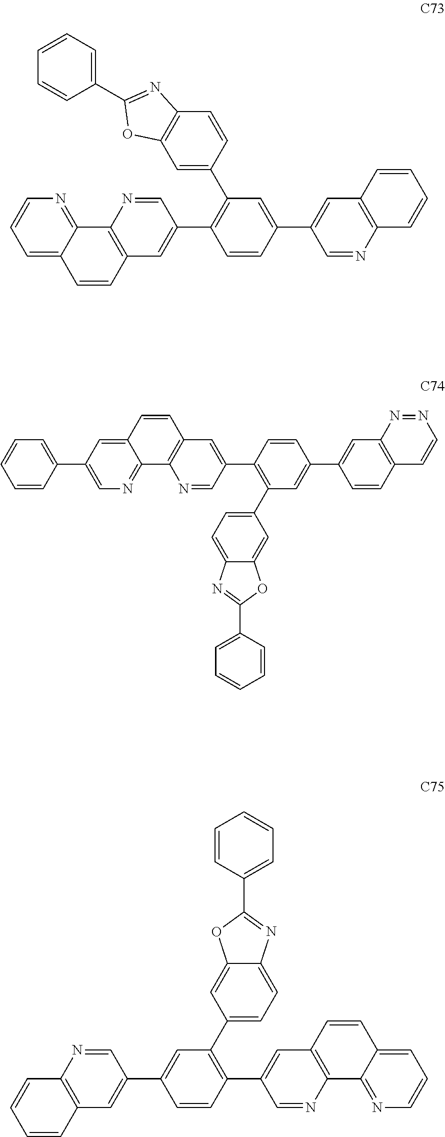

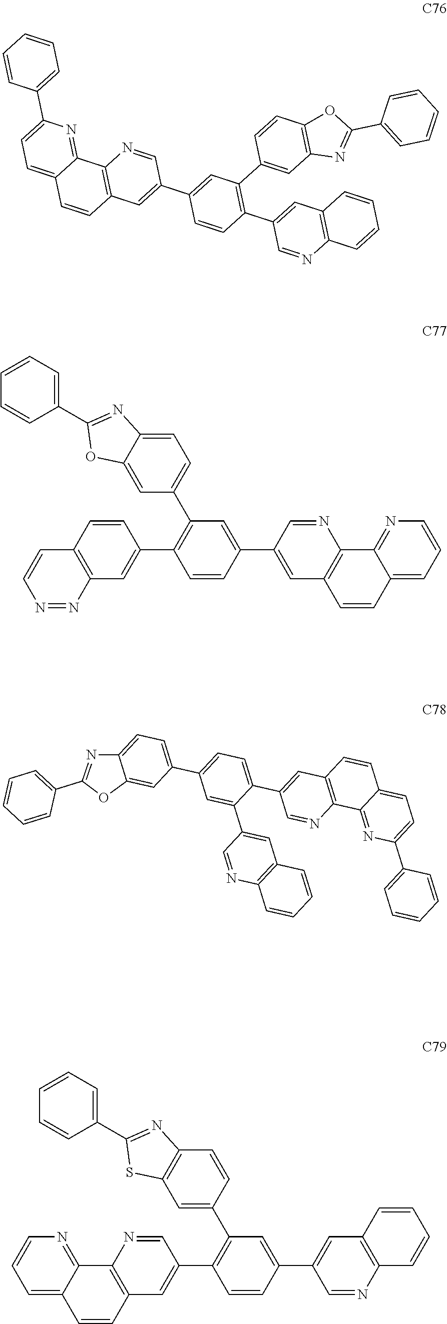

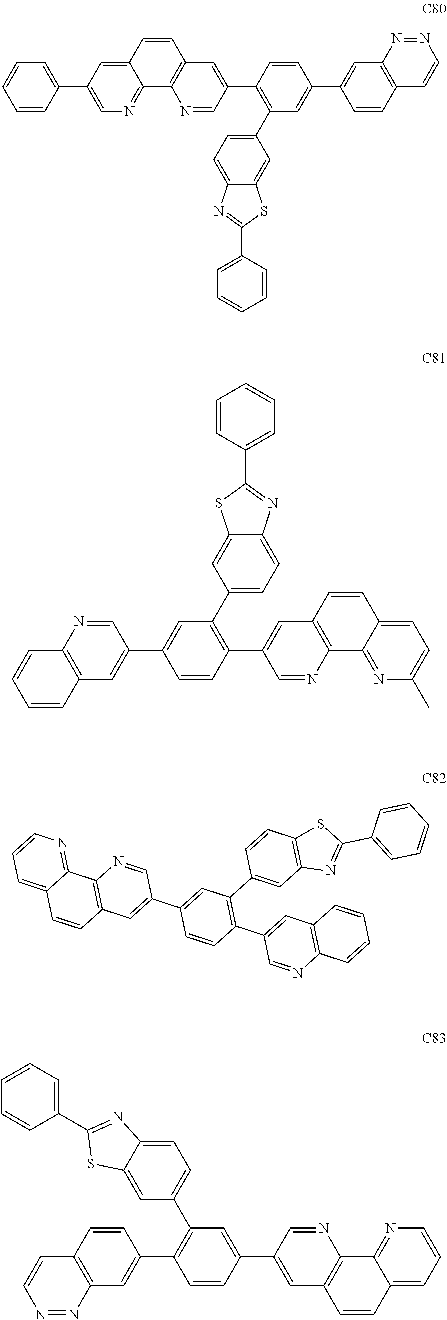

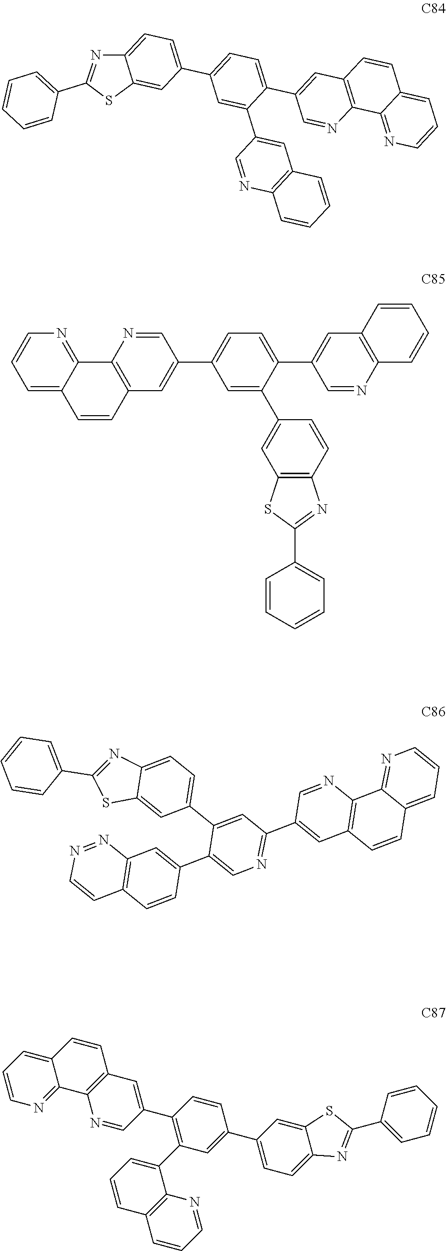

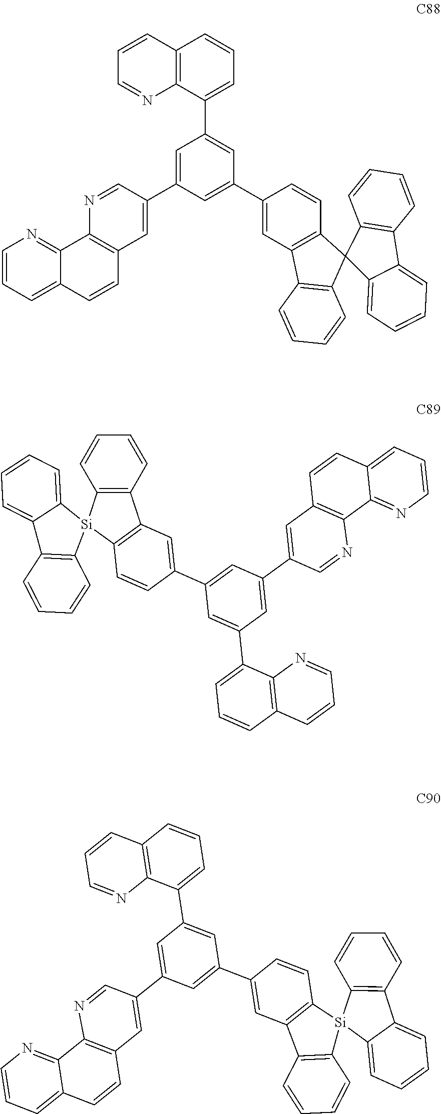

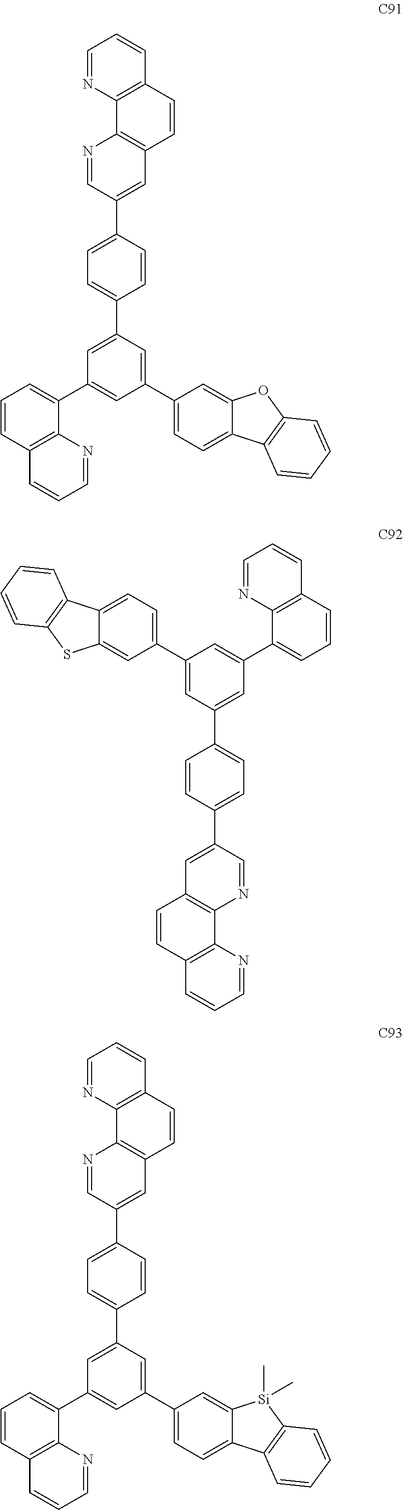

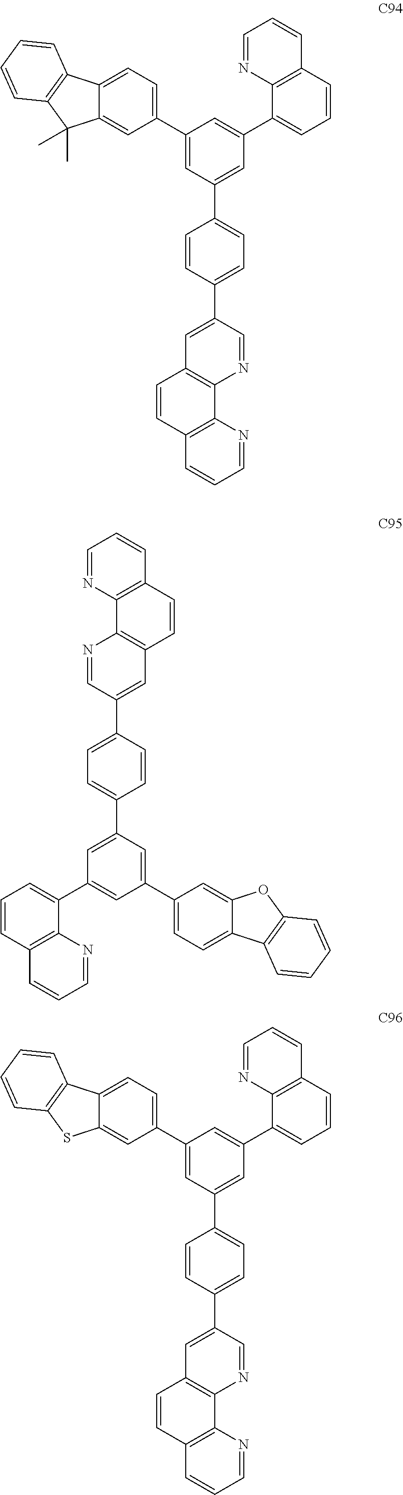

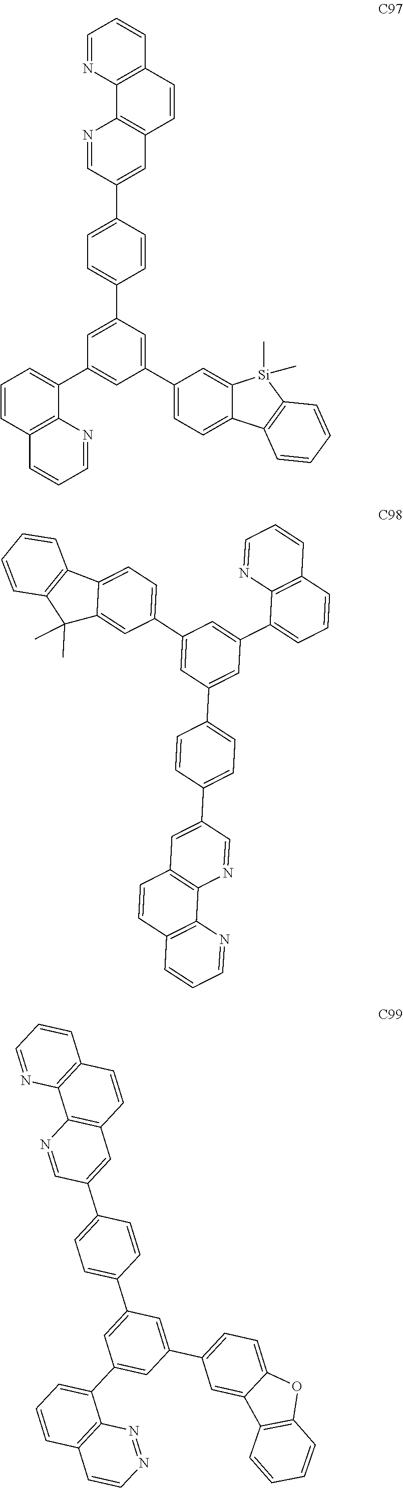

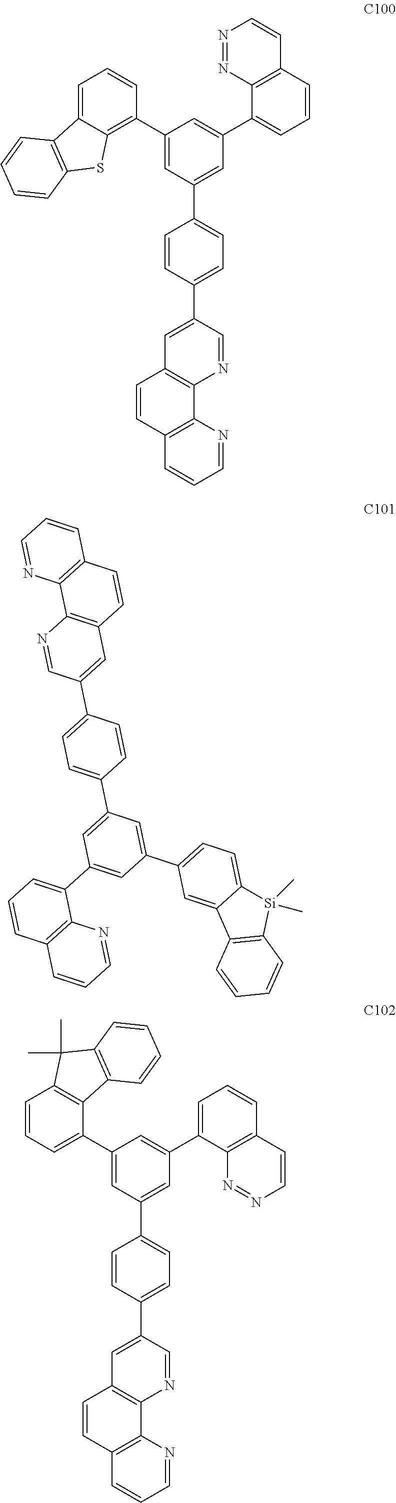

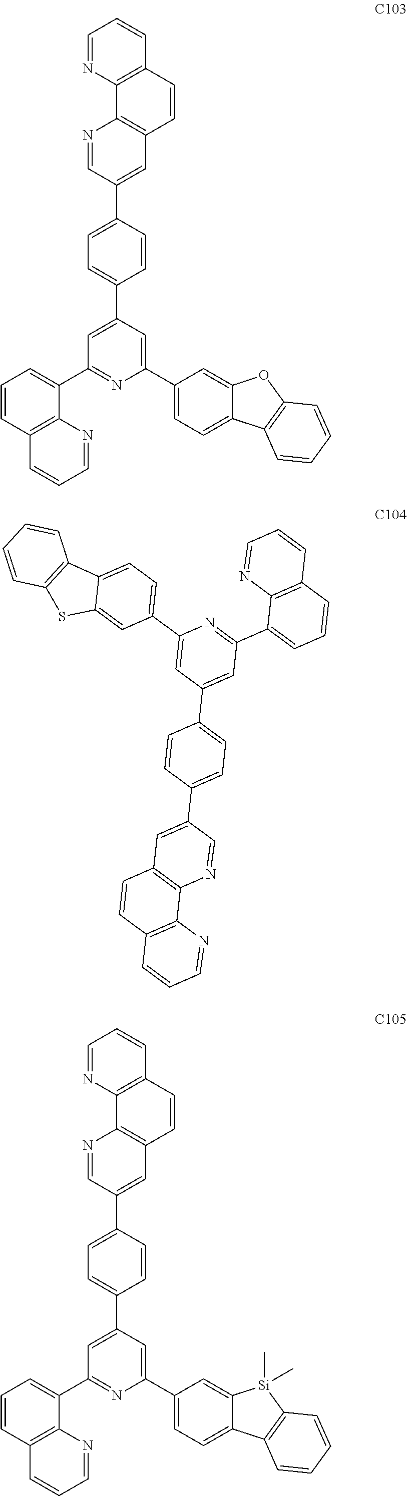

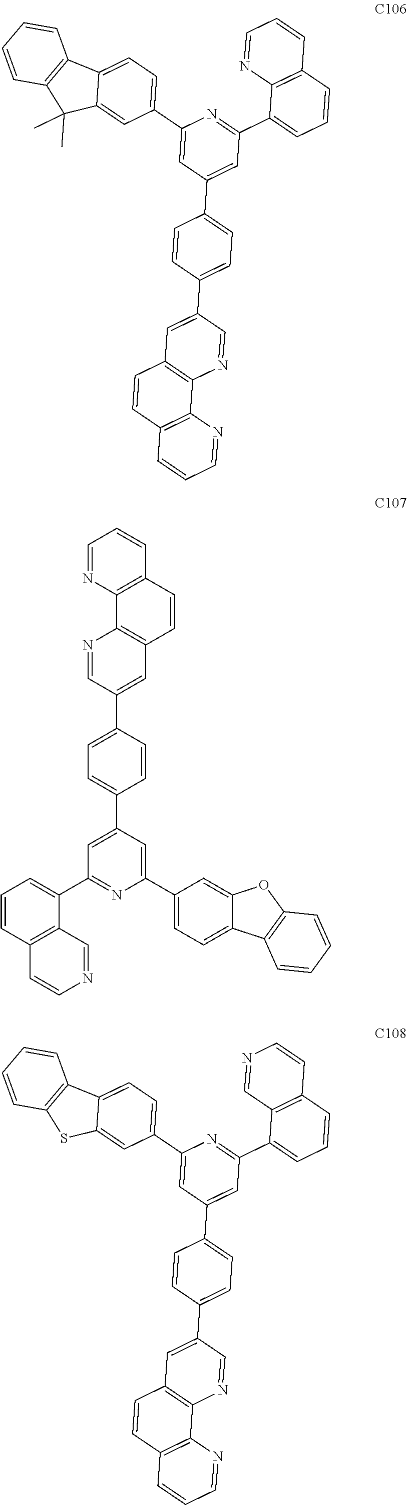

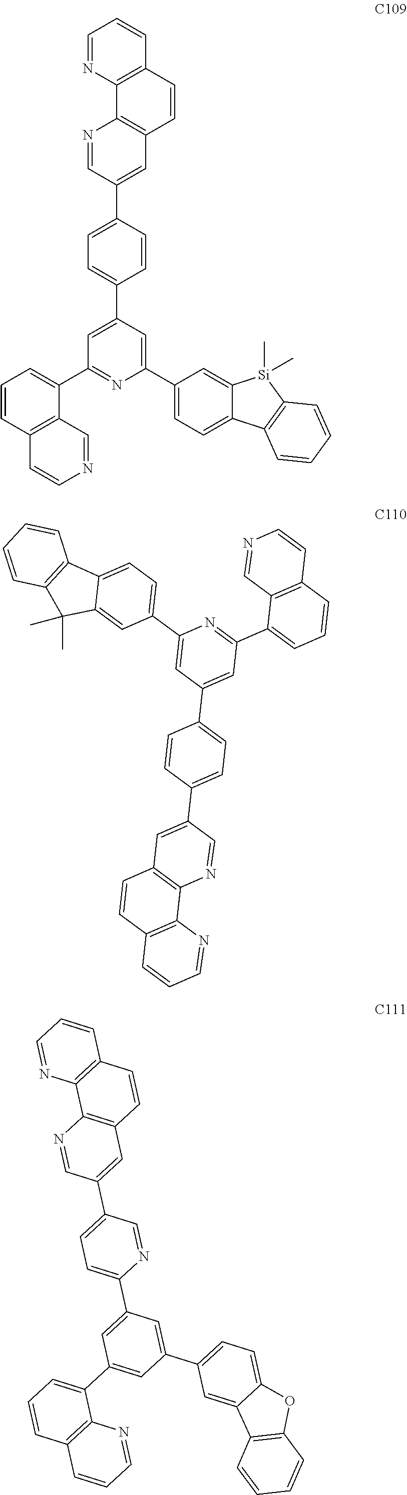

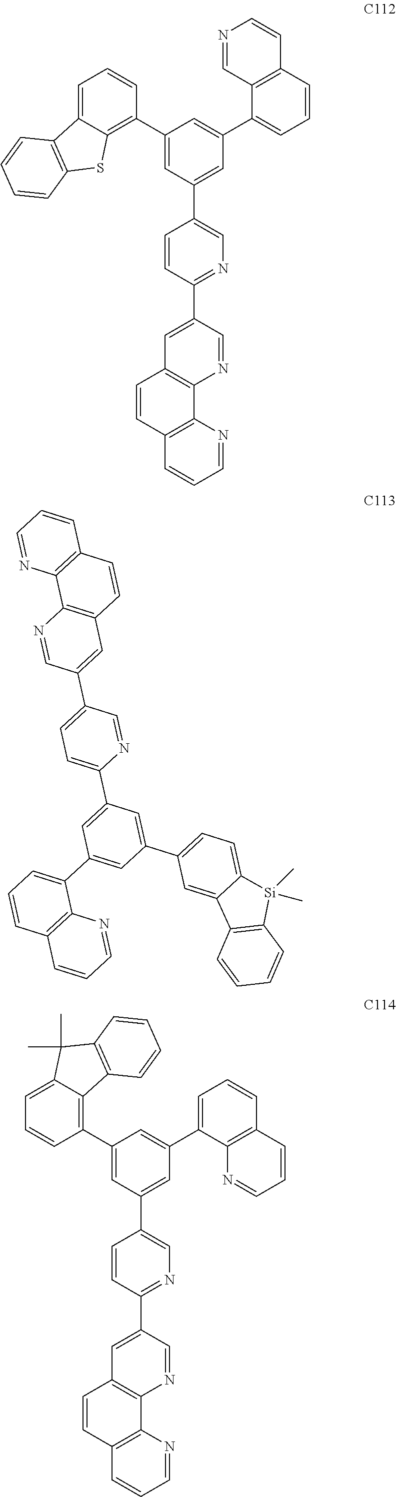

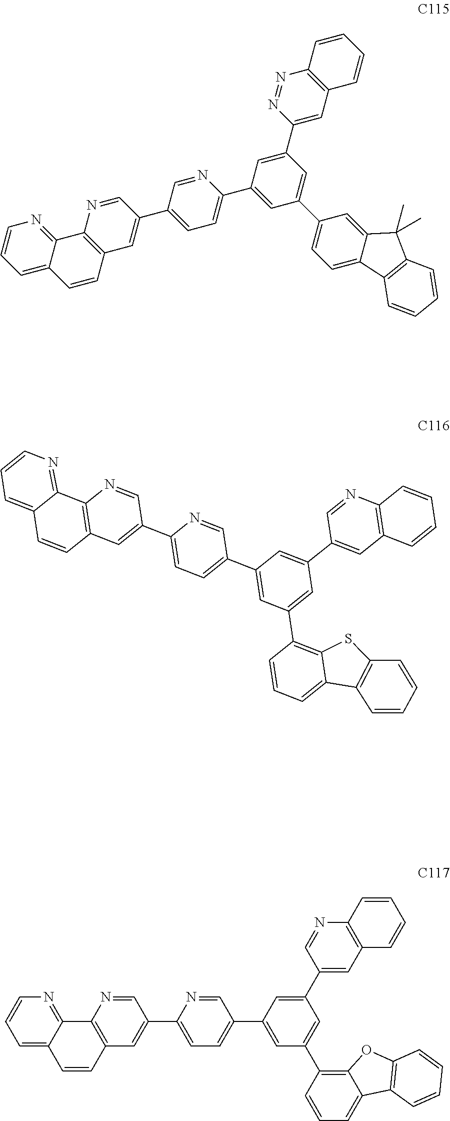

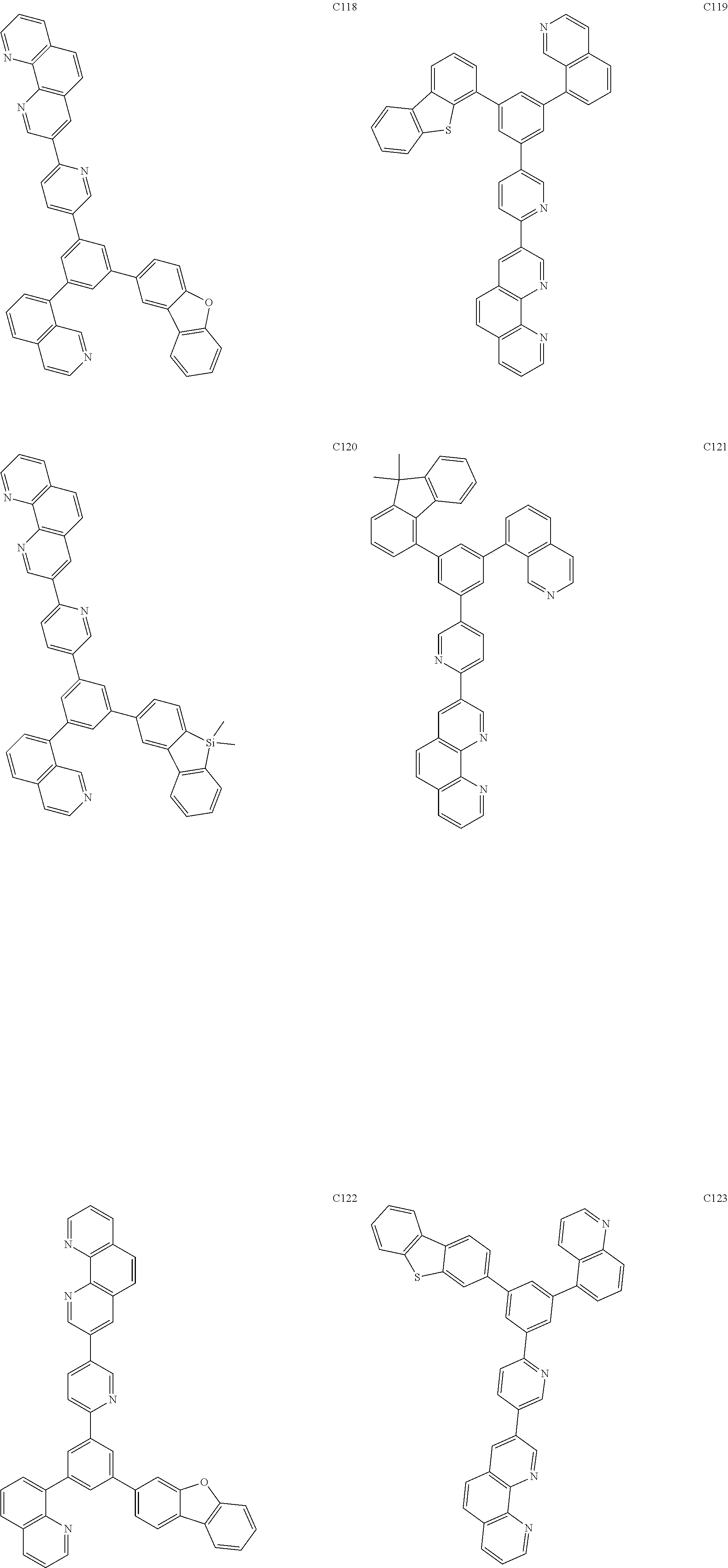

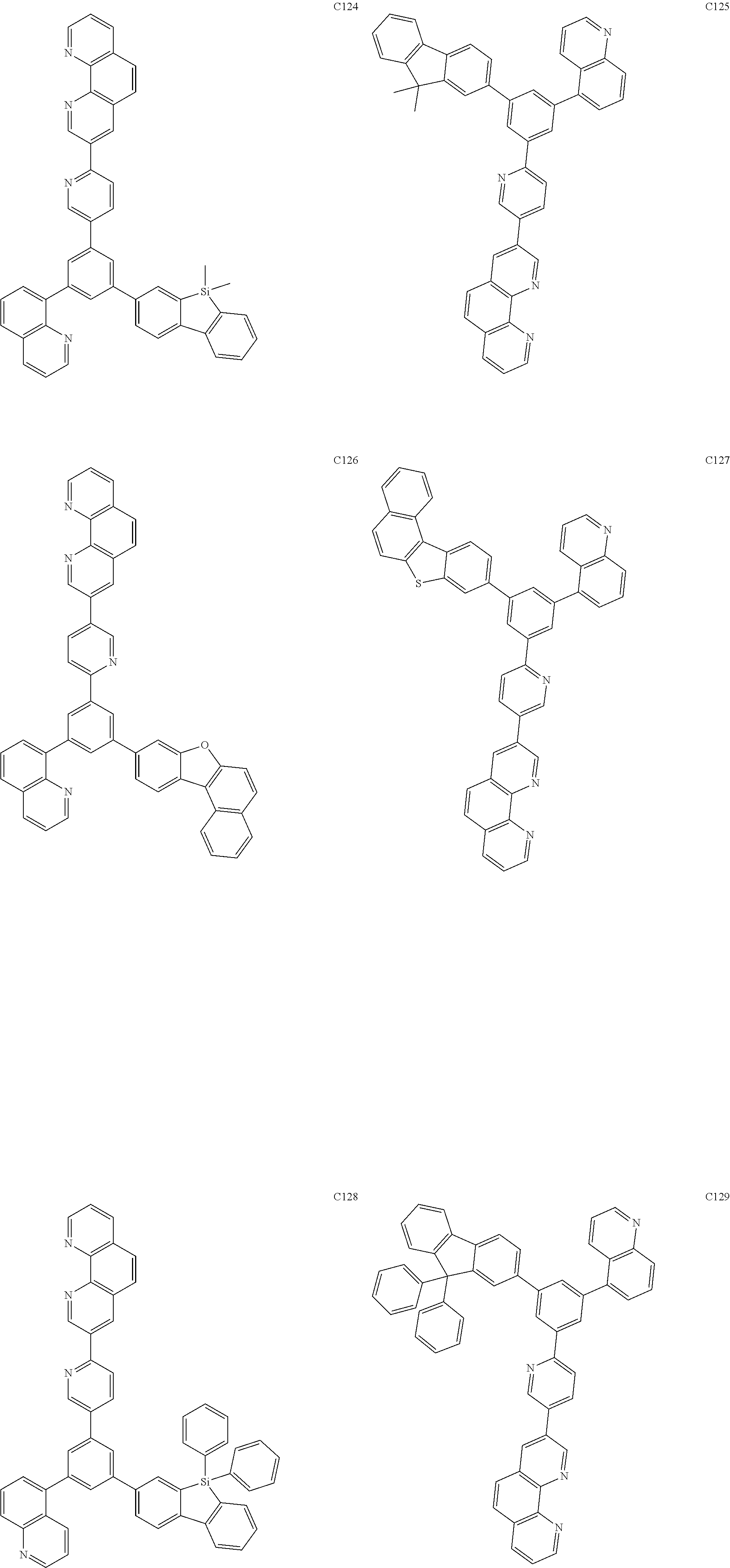

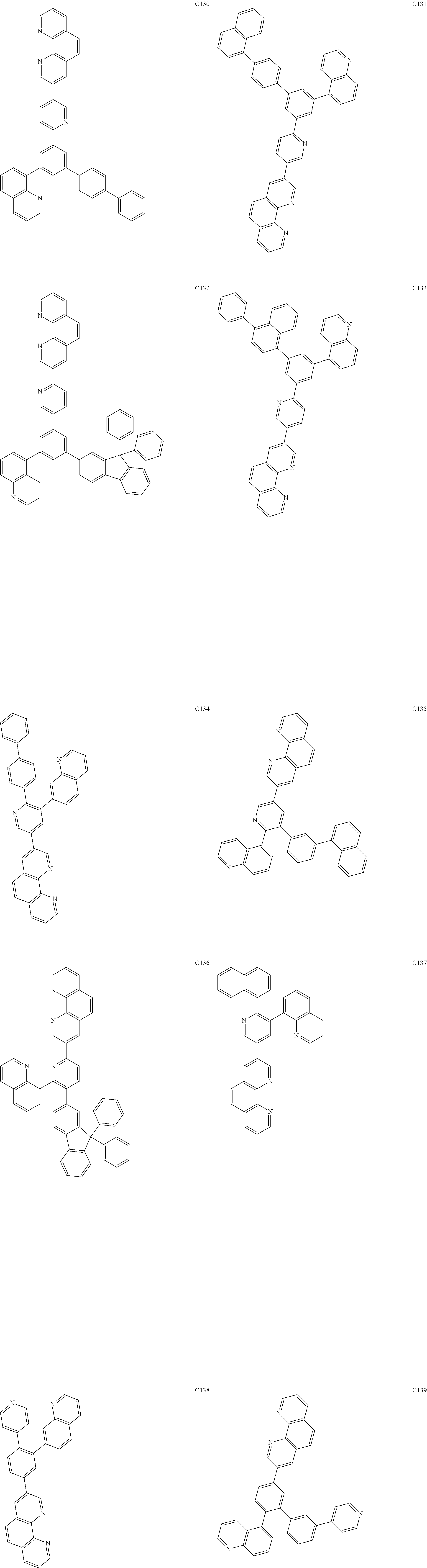

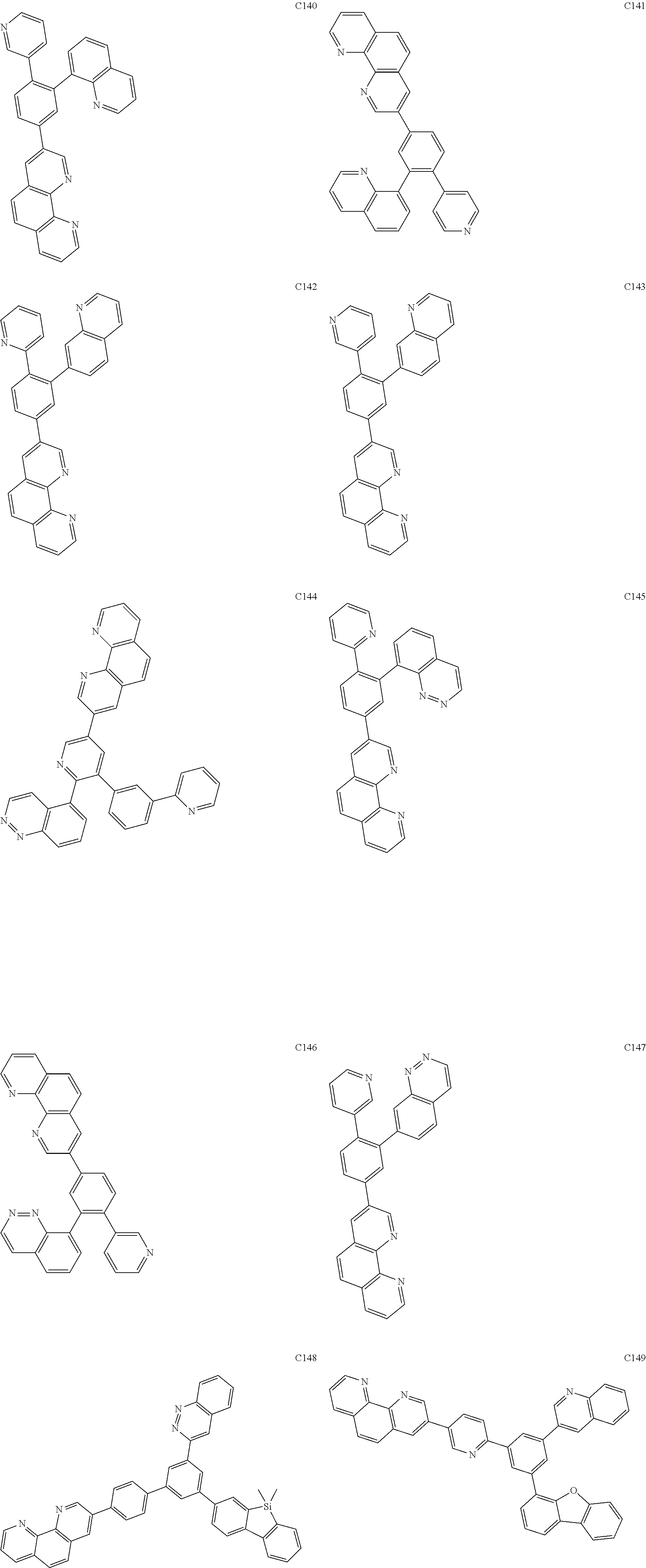

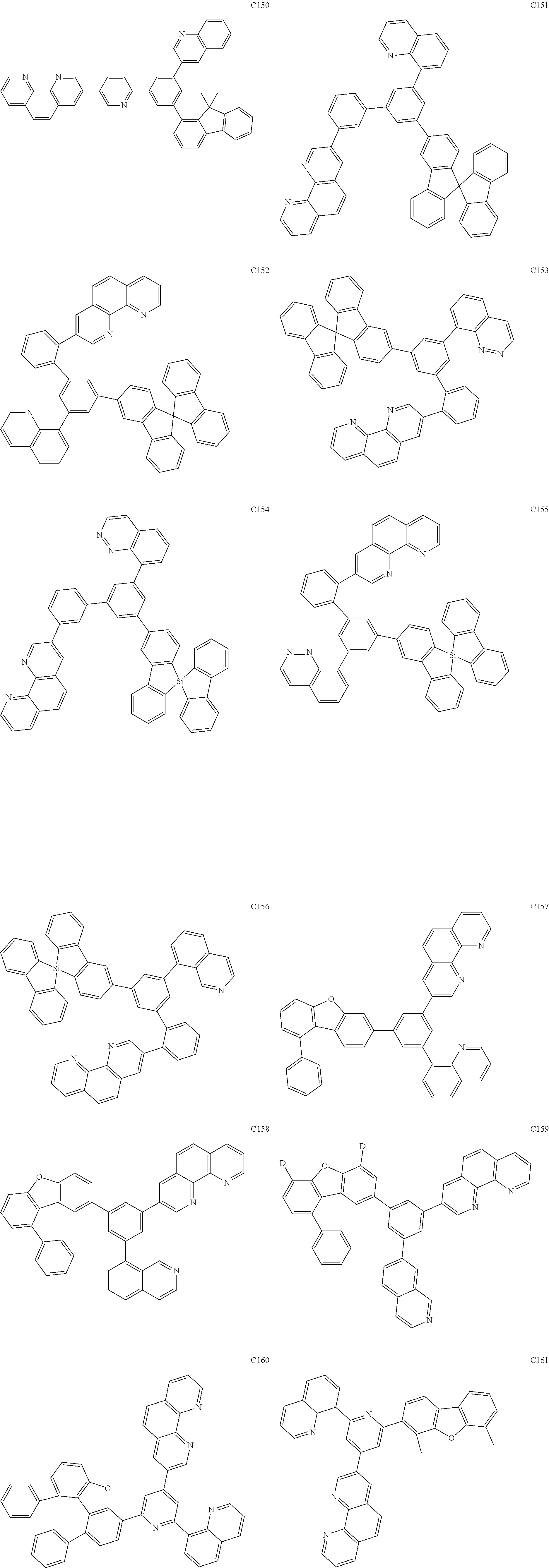

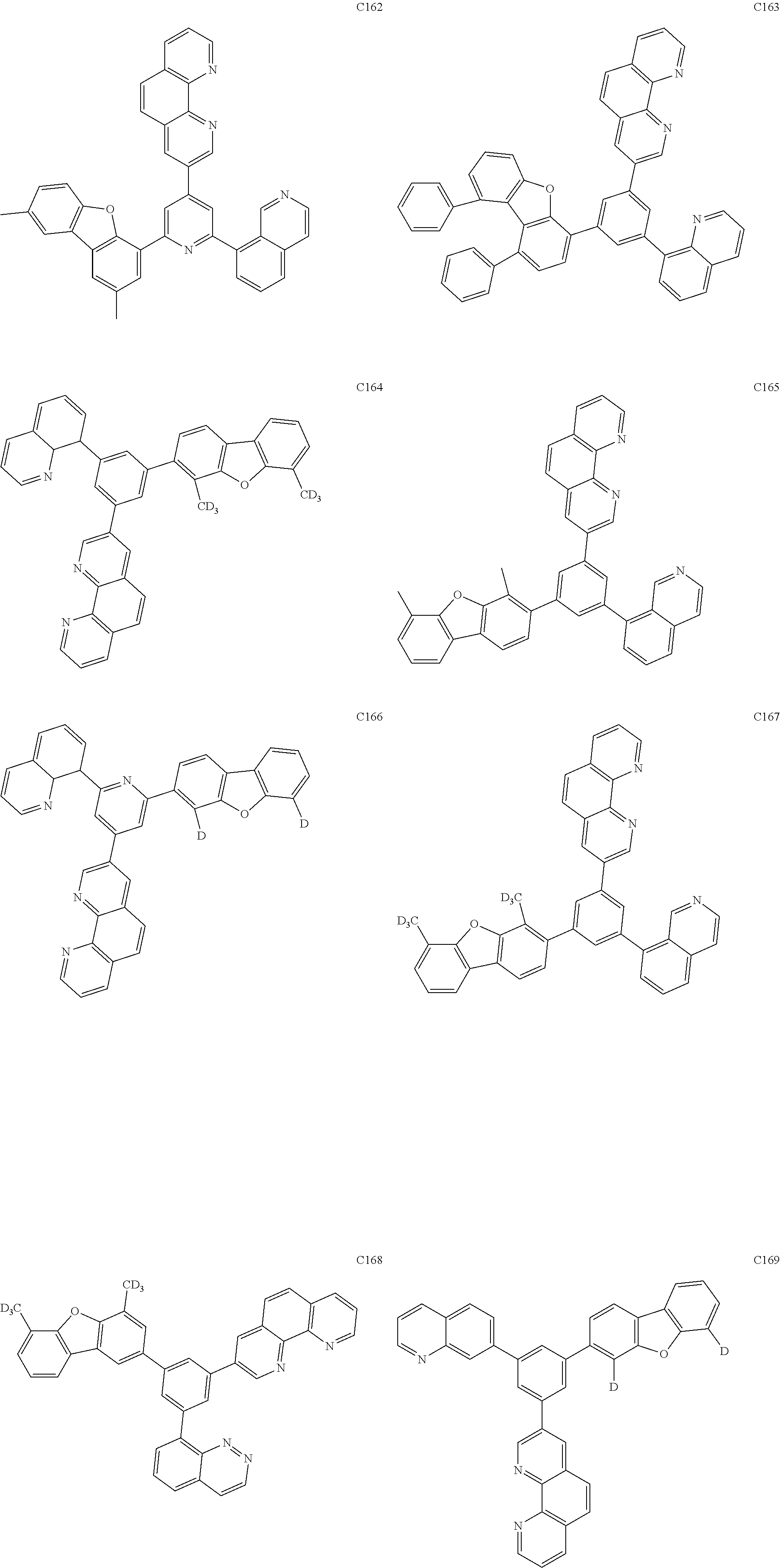

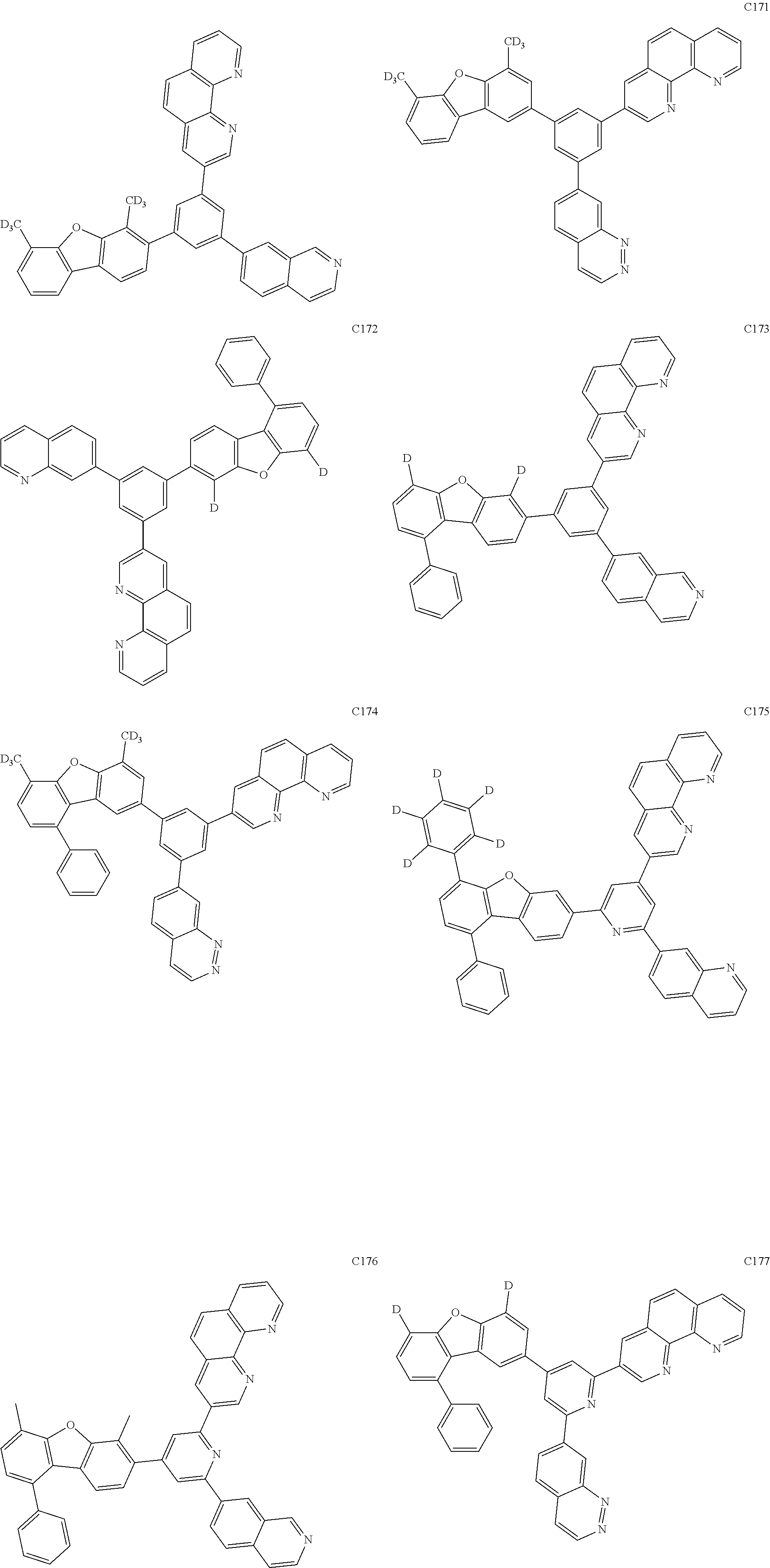

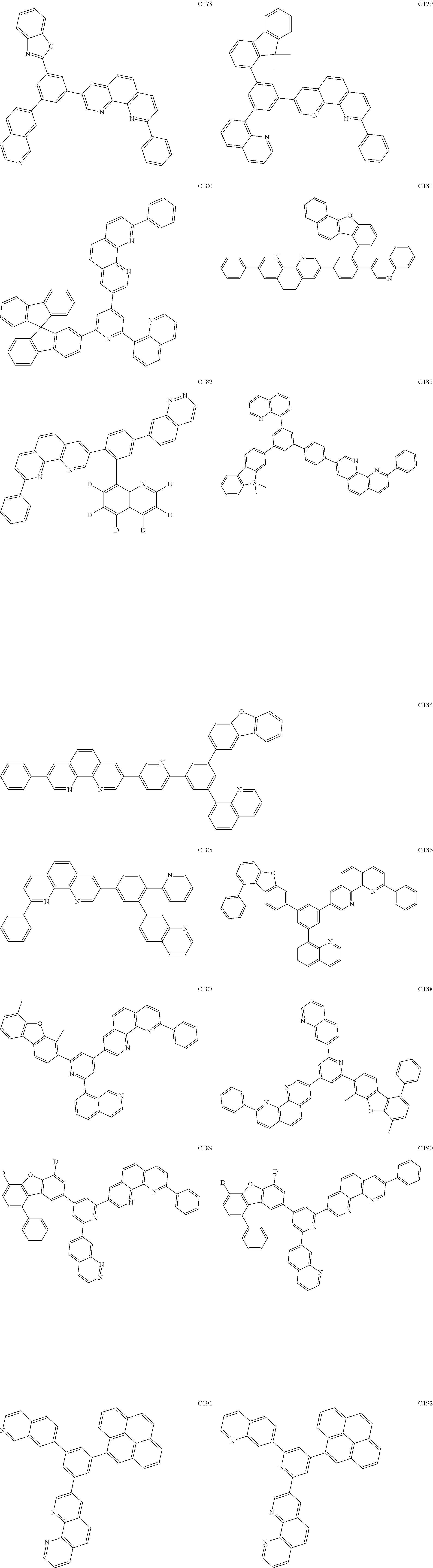

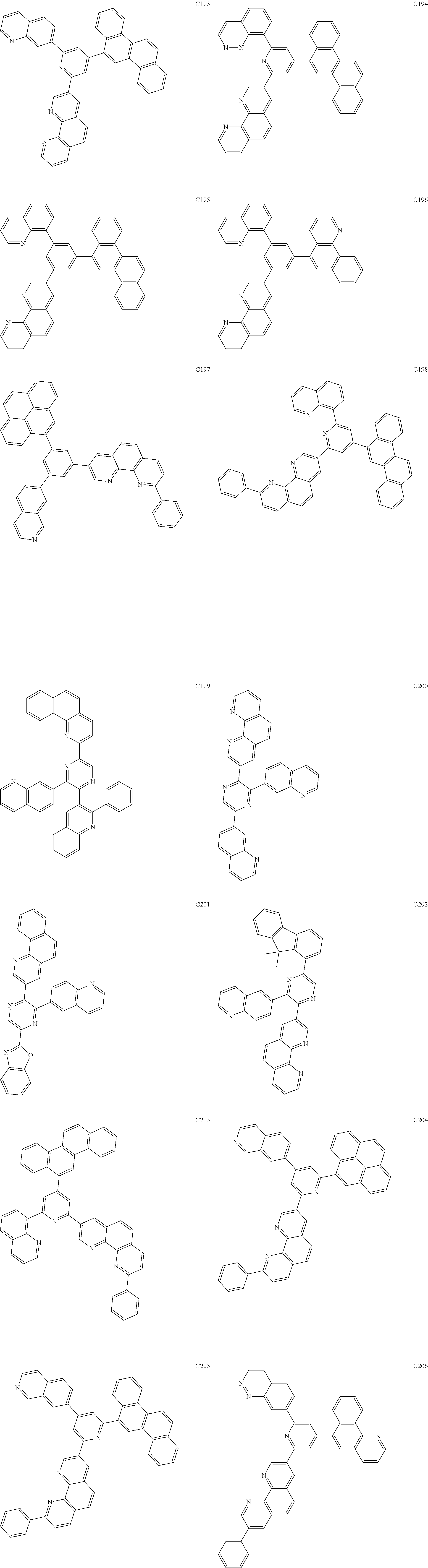

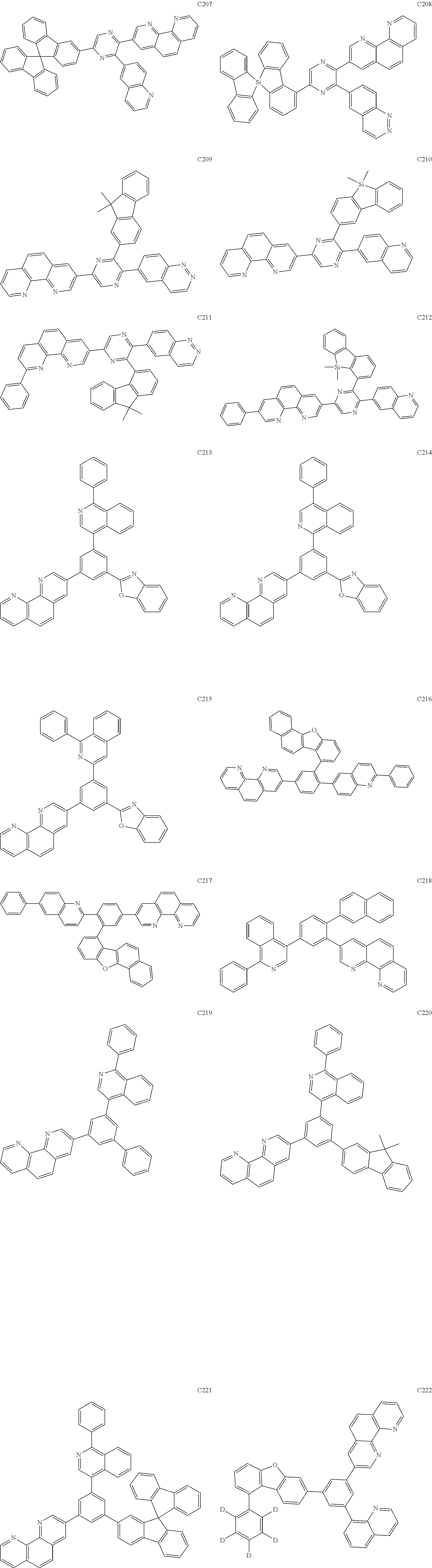

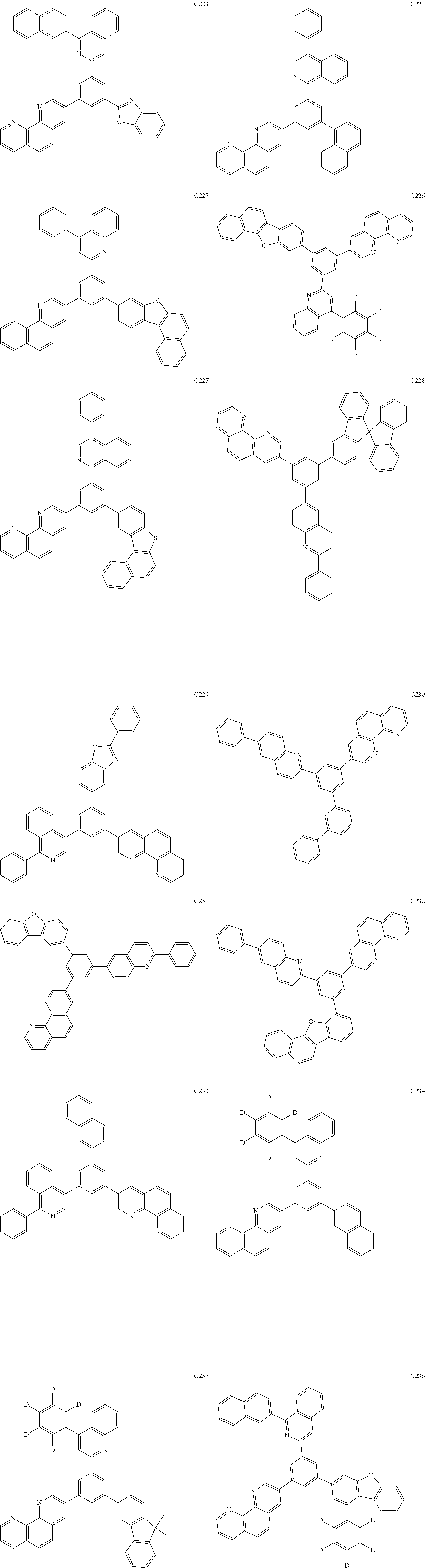

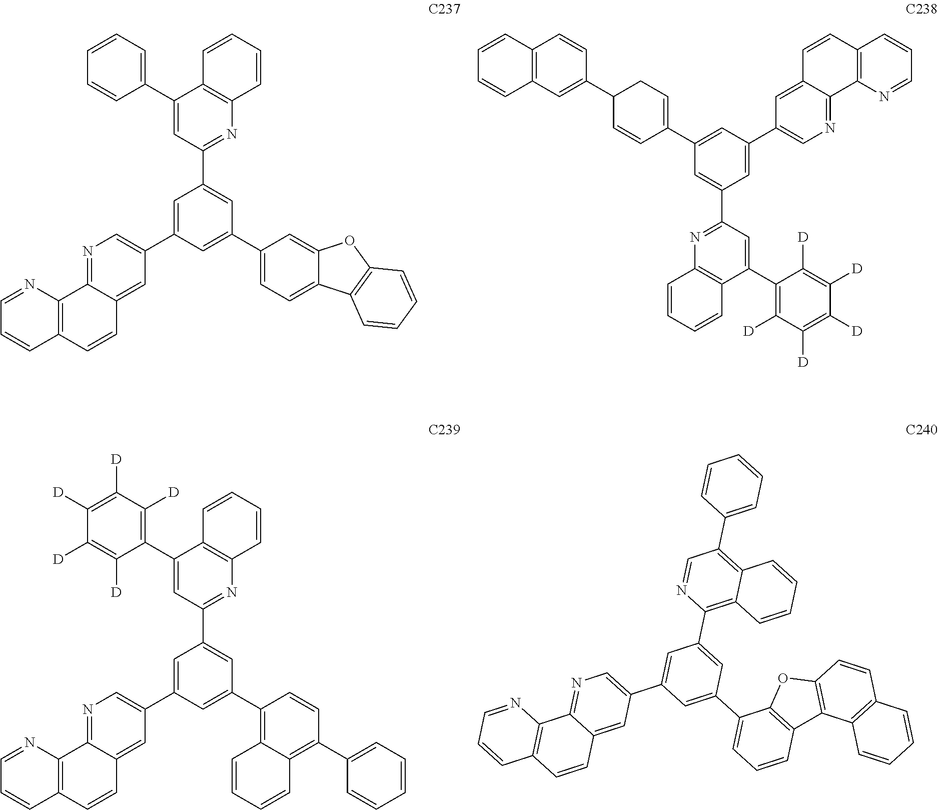

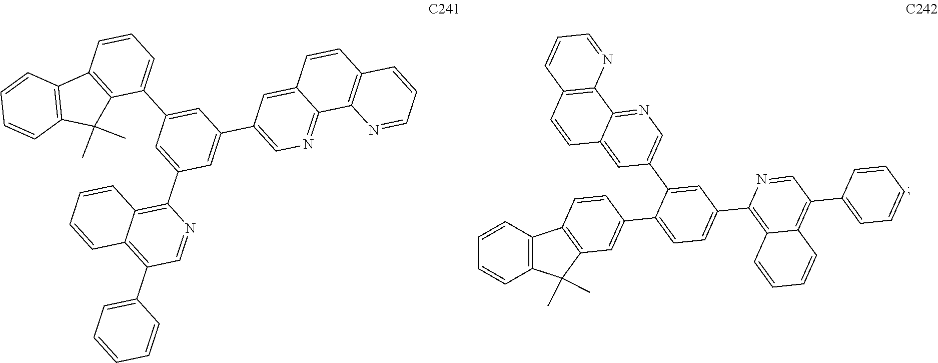

wherein D represents deuterium.

A second aspect of the present disclosure is to provide a charge generation layer material. The charge generation layer material includes the organic compound described in the first aspect.

A third aspect of the present disclosure is to provide an electron transport layer material. The electron transport layer material includes the organic compound described in the first aspect.

A fourth aspect of the present disclosure is to provide an OLED device. The OLED device includes an anode, a cathode, at least two organic light-emitting units disposed between the anode and cathode, and a charge generation layer connected to adjacent light-emitting units, wherein the charge generation layer includes the organic compound described in the first aspect.

A fifth aspect of the present disclosure is to provide an OLED device. The OLED device includes an anode, a cathode, at least two organic light-emitting units disposed between the anode and cathode, and a charge generation layer connected to adjacent light-emitting units, wherein each of the at least two organic light-emitting units includes the organic compound described in the first aspect.

In some preferred embodiments, each of the at least two organic light-emitting units includes any one or a combination of at least two of a hole injection layer, a hole transport layer, a light-emitting layer, an electron blocking layer, a hole blocking layer, an electron transport layer and an electron injection layer.

In some preferred embodiments, each of the at least two organic light-emitting units includes an electron transport layer, and the electron transport layer includes the organic compound described in the first aspect.

In the OLED device provided by the present disclosure, the material of the anode may be a metal, a metal oxide or a conductive polymer, wherein the metal includes copper, gold, silver, iron, chromium, nickel, manganese, palladium, platinum, etc. as well as alloys thereof, the metal oxide includes indium tin oxide (ITO), indium zinc oxide (IZO), zinc oxide, indium gallium zinc oxide (IGZO), etc., and the conductive polymer includes polyaniline, polypyrrole, poly(3-methylthiophene), etc. In addition to the above materials that facilitate hole injection and combinations thereof, the material of the anode further includes known materials suitable for the anode.

In the OLED device, the material of the cathode may be a metal or a multilayer metal material, wherein the metal includes aluminum, magnesium, silver, indium, tin, titanium, etc. as well as alloys thereof, and the multilayer metal material includes LiF/Al, LiO₂/Al, BaF₂/Al, etc. In addition to the above materials that facilitate electron injection and combinations thereof, the material of the cathode further includes known materials suitable for the cathode.

In the OLED device, the organic thin-film layer includes at least one light-emitting layer (EML) and any one or a combination of at least two of a hole injection layer (HIL), a hole transport layer (HTL), an electron blocking layer (EBL), a hole blocking layer (HBL), an electron transport layer (ETL) or an electron injection layer (EIL) that are arranged on two sides of the at least one light-emitting layer.

The OLED device may be prepared by the following method: forming an anode on a transparent or opaque smooth substrate, forming organic thin layers on the anode, and forming a cathode on the organic thin layers. The organic thin layers may be formed by using known film forming methods such as evaporation, sputtering, spin coating, impregnation, and ion plating.

A sixth aspect of the present disclosure is to provide a display panel. The display panel includes the OLED device described in the fourth aspect.

A seventh aspect of the present disclosure is to provide an organic light-emitting display device. The organic light-emitting display device includes the display panel described in the sixth aspect.

An eighth aspect of the present disclosure is to provide an electronic device. The electronic device includes the display panel described in the seventh aspect.

Several preparation examples of the organic compound of the present disclosure are exemplarily described below.

### EXAMPLE 1 COMPOUND C01

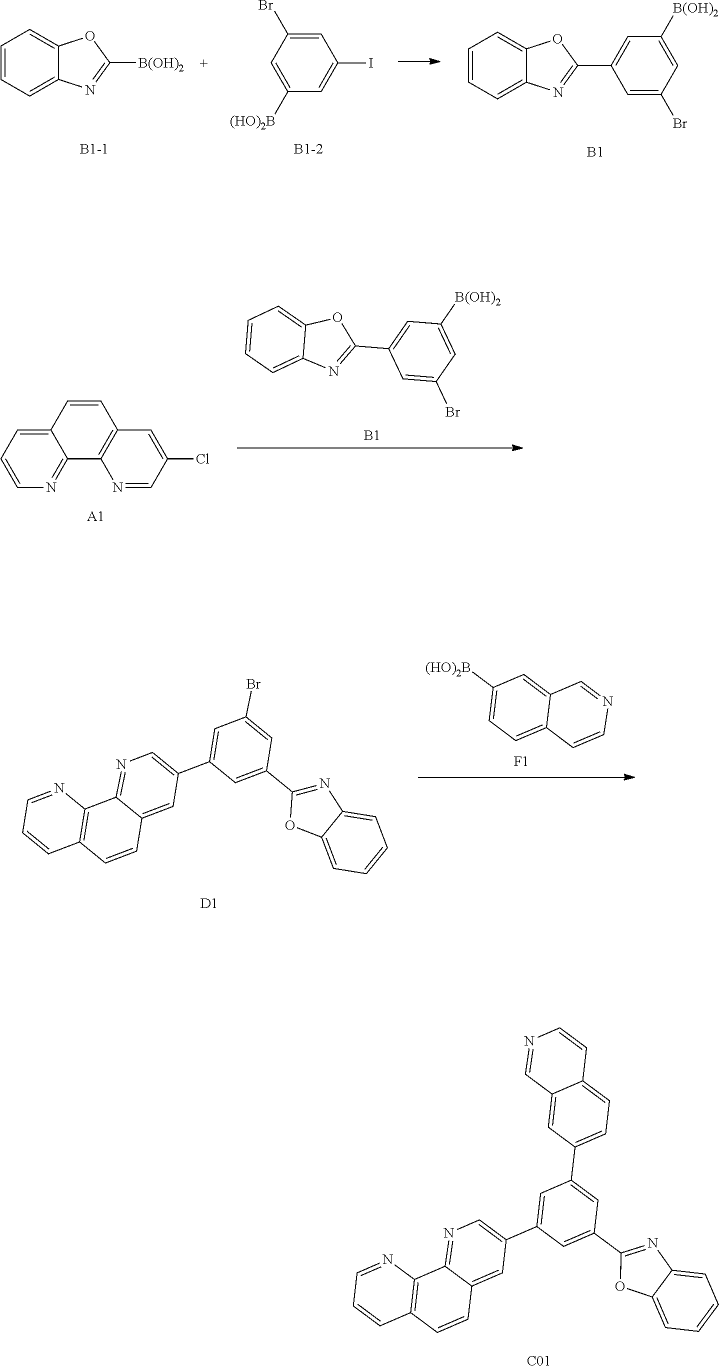

Synthesis of Reactant B1: Nitrogen was introduced in a 250 mL three-necked flask, 0.05 mol of raw material B1-1, 100 mL of THF, 0.04 mol of raw material B1-2 and 0.0004 mol of tetrakis(triphenylphosphine)palladium were added and stirred, 0.06 mol of K₂CO₃ aqueous solution (2 M) was added, and the resulting mixture was heated to 75° C. and reacted at reflux for 10 h. The samples were collected and subjected to TLC until the reaction was completed. The reaction was naturally cooled. The reaction product was extracted with 200 mL of dichloromethane, the layers were separated, and the extract liquor was dried with anhydrous sodium sulfate and filtered. The filtrate was rotary evaporated and purified by a silica gel column to give the reactant B1. The elemental analysis results were as follows: molecular formula: C₁₃H₉BBrNO₃, Theoretical: C, 49.11; H, 2.85; B, 3.40; Br, 25.13; N, 4.41; 0, 15.10; Found: C, 49.13; H, 2.86; B, 3.40; Br, 25.12; N, 4.40; O, 15.10. m/z: Calc: 316.99, Found: 317.26.

Synthesis of Intermediate D1: In a 250 mL round-bottom flask, reactant A1 (10 mmol), reactant B1 (12 mmol) and Na₂CO₃ (80 mmol) were added to toluene/EtOH (anhydrous ethanol)/H₂O (75/25/50, mL) solvent respectively to obtain a mixed solution. Then Pd(PPh₃)₄ (0.48 mmol) was added to the above mixed solution and reacted at reflux for 20 h in a nitrogen atmosphere to give an intermediate. The obtained intermediate was cooled to room temperature, added to water, filtered through a Celite pad, extracted with dichloromethane, then washed with water, dried with anhydrous magnesium sulfate, filtered, evaporated, and purified by silica gel column chromatography to give the intermediate D1. The elemental analysis results were as follows: molecular formula: C₂₅H₁₄BrN₃O, Theoretical: C, 66.39; H, 3.12; Br, 17.67; N, 9.29; O, 3.54; Found: C, 66.41; H, 3.11; Br, 17.67; N, 9.29; O, 3.54. m/z: Calc: 451.03, Found: 451.40.

Synthesis of Product C01: In a 250 mL round-bottom flask, intermediate D1 (10 mmol), reactant F1 (12 mmol) and Na₂CO₃ (80 mmol) were added to toluene/EtOH (anhydrous ethanol)/H₂O (75/25/50, mL) solvent respectively to obtain a mixed solution. Then Pd(PPh₃)₄ (0.48 mmol) was added to the above mixed solution and reacted at reflux for 20 h in a nitrogen atmosphere to give an intermediate. The obtained intermediate was cooled to room temperature, added to water, filtered through a Celite pad, extracted with dichloromethane, then washed with water, dried with anhydrous magnesium sulfate, filtered, evaporated, and purified by silica gel column chromatography to give the final product C01.

MALDI-TOF: m/z: Calc for C₃₄H₂₀N₄O: 500.16, Found: 500.37.

The elemental analysis results of the compound were as follows: Calc for C₃₄H₂₀N₄O(%): C, 81.58; H, 4.03; N, 11.19; O, 3.20; Found: C, 81.59; H, 4.02; N, 11.18; O , 3.20.

### EXAMPLE 2 COMPOUND C07

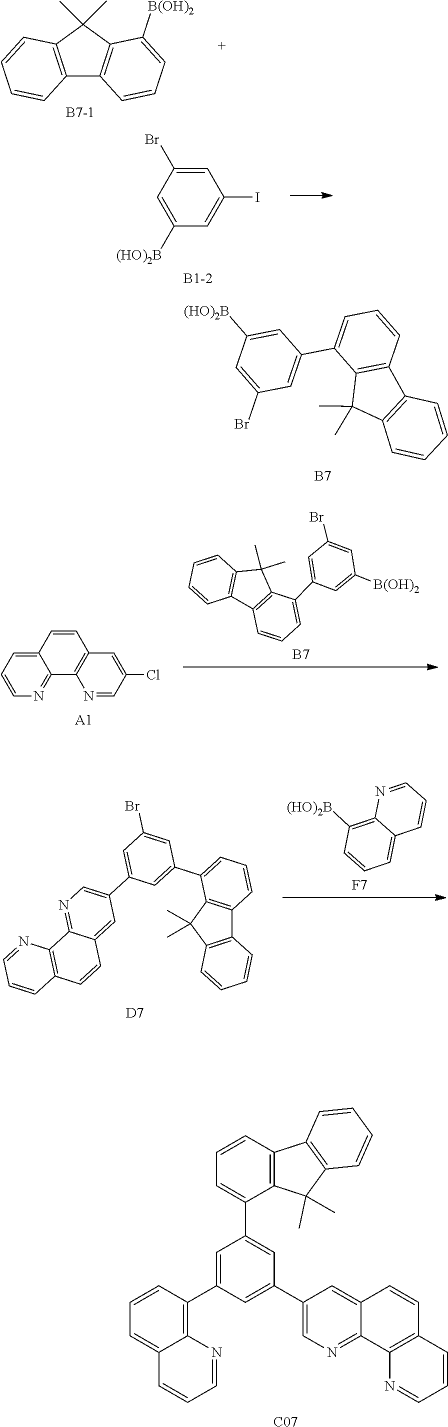

Synthesis of Reactant B7: Nitrogen was introduced in a 250 mL three-necked flask, 0.05 mol of raw material B7-1, 100 mL of THE, 0.04 mol of raw material B1-2 and 0.0004 mol of tetrakis(triphenylphosphine)palladium were added and stirred, 0.06 mol of K₂CO₃ aqueous solution (2 M) was added, and the resulting mixture was heated to 78° C. and reacted at reflux for 10 h. The samples were collected and subjected to TLC until the reaction was completed. The reaction was naturally cooled. The reaction product was extracted with 200 mL of dichloromethane, the layers were separated, and the extract liquor was dried with anhydrous sodium sulfate and filtered. The filtrate was rotary evaporated and purified by a silica gel column to give the reactant B7. The elemental analysis results were as follows: molecular formula: C₂₁H₁₈BBrO₂, Theoretical: C, 64.17; H, 4.62; B, 2.75; Br, 20.33; O, 8.14; Found: C, 64.15; H, 4.63; B, 2.75; Br, 20.34; O, 8.14. m/z: Calc: 392.06, Found: 392.47.

Synthesis of Intermediate D7: In a 250 mL round-bottom flask, reactant A1 (10 mmol), reactant B7 (12 mmol) and Na₂CO₃ (80 mmol) were added to toluene/EtOH (anhydrous ethanol)/H₂O (75/25/50, mL) solvent respectively to obtain a mixed solution. Then Pd(PPh₃)₄ (0.48 mmol) was added to the above mixed solution and reacted at reflux for 20 h in a nitrogen atmosphere to give an intermediate. The obtained intermediate was cooled to room temperature, added to water, filtered through a Celite pad, extracted with dichloromethane, then washed with water, dried with anhydrous magnesium sulfate, filtered, evaporated, and purified by silica gel column chromatography to give the intermediate D7. The elemental analysis results were as follows: molecular formula: C₃₃H₂₃BrN₂, Theoretical: C, 75.14; H, 4.40; Br, 15.15; N, 5.31; Found: C, 75.12; H, 4.41; Br, 15.16; N, 5.31. m/z: Calc: 526.10, Found: 526.39.

Synthesis of Product C07: In a 250 mL round-bottom flask, intermediate D7 (10 mmol), reactant F7 (12 mmol) and Na₂CO₃ (80 mmol) were added to toluene/EtOH (anhydrous ethanol)/H₂O (75/25/50, mL) solvent respectively to obtain a mixed solution.

Then Pd(PPh₃)₄ (0.48 mmol) was added to the above mixed solution and reacted at reflux for 20 h in a nitrogen atmosphere to give an intermediate. The obtained intermediate was cooled to room temperature, added to water, filtered through a Celite pad, extracted with dichloromethane, then washed with water, dried with anhydrous magnesium sulfate, filtered, evaporated, and purified by silica gel column chromatography to give the final product C07. MALDI-TOF (m/z): Calc for C₄₂H₂₉N₃: 575.24; Found: 575.68.

The elemental analysis results of the compound were as follows: Calc for C₄₂H₂₉N₃ (%): C, 87.62; H, 5.08; N, 7.30; Found: C, 87.64; H, 5.07; N, 7.30.

### EXAMPLE 3 COMPOUND C18

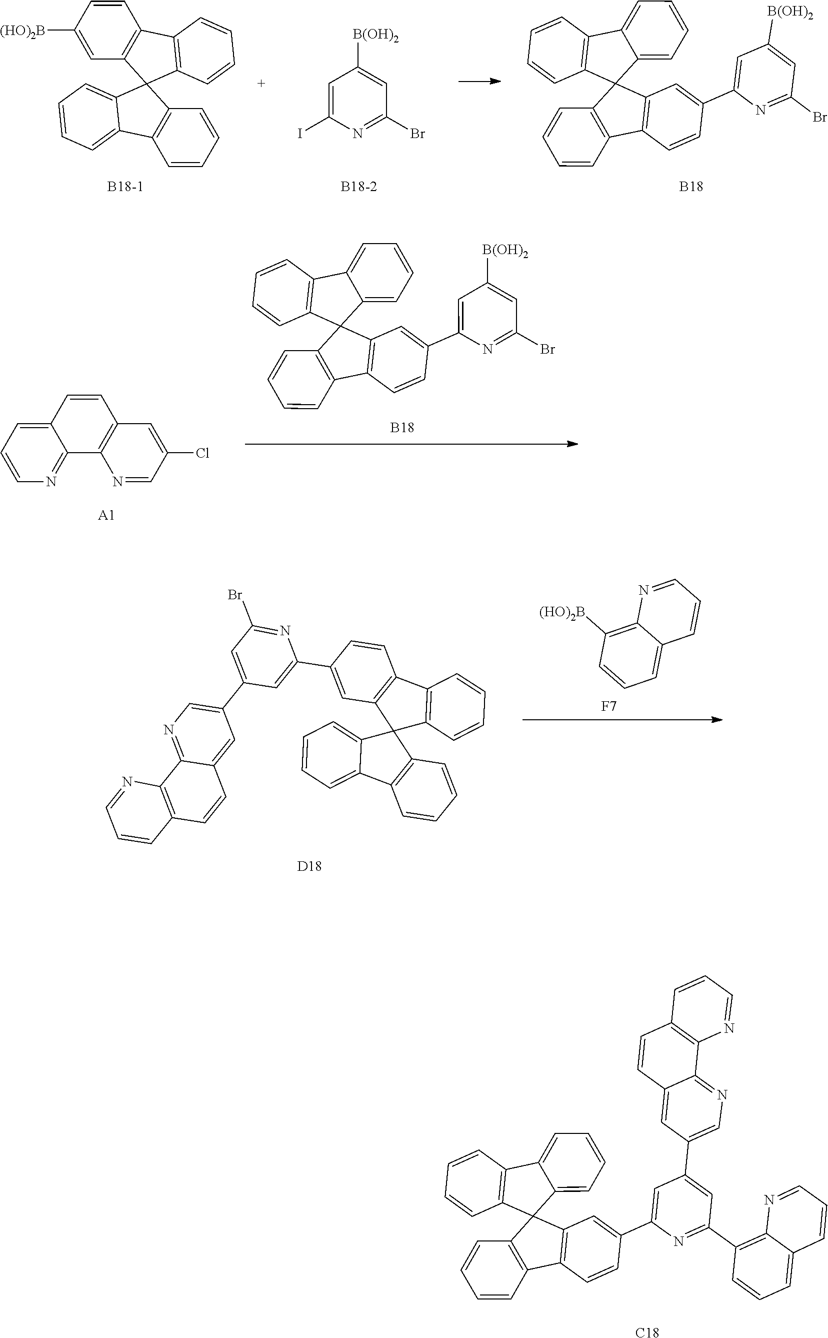

Synthesis of Reactant B18: Nitrogen was introduced in a 250 mL three-necked flask, 0.05 mol of raw material B18-1, 100 mL of THF, 0.04 mol of raw material B18-2 and 0.0004 mol of tetrakis(triphenylphosphine)palladium were added and stirred, 0.06 mol of K₂CO₃ aqueous solution (2 M) was added, and the resulting mixture was heated to 80° C. and reacted at reflux for 10 h. The samples were collected and subjected to TLC until the reaction was completed. The reaction was naturally cooled. The reaction product was extracted with 200 mL of dichloromethane, the layers were separated, and the extract liquor was dried with anhydrous sodium sulfate and filtered. The filtrate was rotary evaporated and purified by a silica gel column to give the reactant B18. The elemental analysis results were as follows: molecular formula: C₁₃H₉BBrNO₃, Theoretical: C, 69.80; H, 3.71; B, 2.09; Br, 15.48; N, 2.71; O, 6.20; Found: C, 69.78; H, 3.72; B, 2.09; Br, 15.49; N, 2.71; O, 6.20. m/z: Calc: 515.07, Found: 515.45.

The synthesis method of Compound C18 is similar to the synthesis method of Compound C07, except that B7 was replaced with an equal molar amount of B18, wherein the elemental analysis results of the resulting D18 were as follows: molecular formula: C₄₂H₂₄BrN₃), Theoretical: C, 77.54; H, 3.72; Br, 12.28; N, 6.46; Found: C, 77.56; H, 3.73; Br, 12.29; N, 6.46; and m/z: Calc: 649.12, Found: 649.38.

C18: MALDI-TOF: m/z: Calc for C₅₁H₃₀N₄: 698.25; Found: 698.56.

The elemental analysis results of the compound were as follows: Calc for C₅₁H₃₀N₄ (%): C, 87.66; H, 4.33; N, 8.02; Found: C, 87.67; H, 4.32; N, 8.01.

### EXAMPLE 4 COMPOUND C31

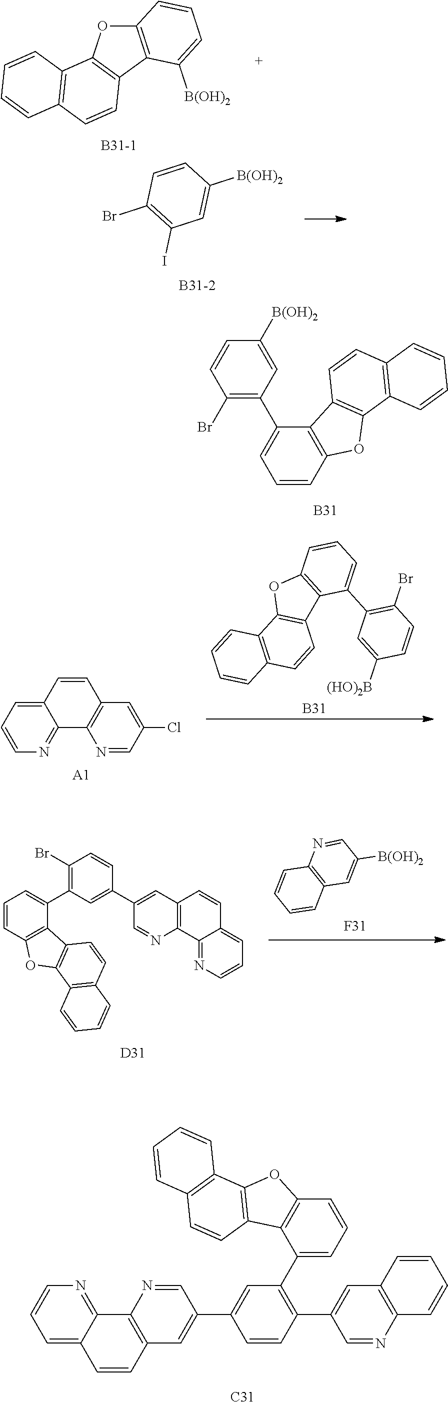

Synthesis of Reactant B31: Nitrogen was introduced in a 250 mL three-necked flask, 0.05 mol of raw material B31-1, 100 mL of THF, 0.04 mol of raw material B31-2 and 0.0004 mol of tetrakis(triphenylphosphine)palladium were added and stirred, 0.06 mol of K₂CO₃ aqueous solution (2 M) was added, and the resulting mixture was heated to 78° C. and reacted at reflux for 10 h. The samples were collected and subjected to TLC until the reaction was completed. The reaction was naturally cooled. The reaction product was extracted with 200 mL of dichloromethane, the layers were separated, and the extract liquor was dried with anhydrous sodium sulfate and filtered. The filtrate was rotary evaporated and purified by a silica gel column to give the reactant B18. The elemental analysis results were as follows: molecular formula: C₂₂H₁₄BBrO₃, Theoretical: C, 63.36; H, 3.38; B, 2.59; Br, 19.16; O, 11.51; Found: C, 63.34; H, 3.39; B, 2.59; Br, 19.16; O, 11.51. m/z: Calc: 416.02, Found: 416.62.

The synthesis method of Compound C31 is similar to the synthesis method of Compound C01, except that B1 was replaced with an equal molar amount of B31, wherein the elemental analysis results of the resulting D31 were as follows: molecular formula: C₃₄H₁₉BrN₂O, Theoretical: C, 74.06; H, 3.47; Br, 14.49; N, 5.08; O, 2.90; Found: C, 74.05; H, 3.47; Br, 14.49; N, 5.08; O, 2.90, and m/z: Calc: 550.07, Found: 550.45; and that F1 in the reaction was replaced with an equal molar amount of F31. C31: MALDI-TOF: m/z: Calc for C₄₃H₂₅N₃₀: 599.20; Found: 599.53.

The elemental analysis results of the compound were as follows: Calc for C₄₃H₂₅N₃₀ (%): C, 86.12; H, 4.20; N, 7.01; O, 2.67; Found: C, 86.13; H, 4.20; N, 7.00; 0, 2.66.

### EXAMPLE 5 COMPOUND C41

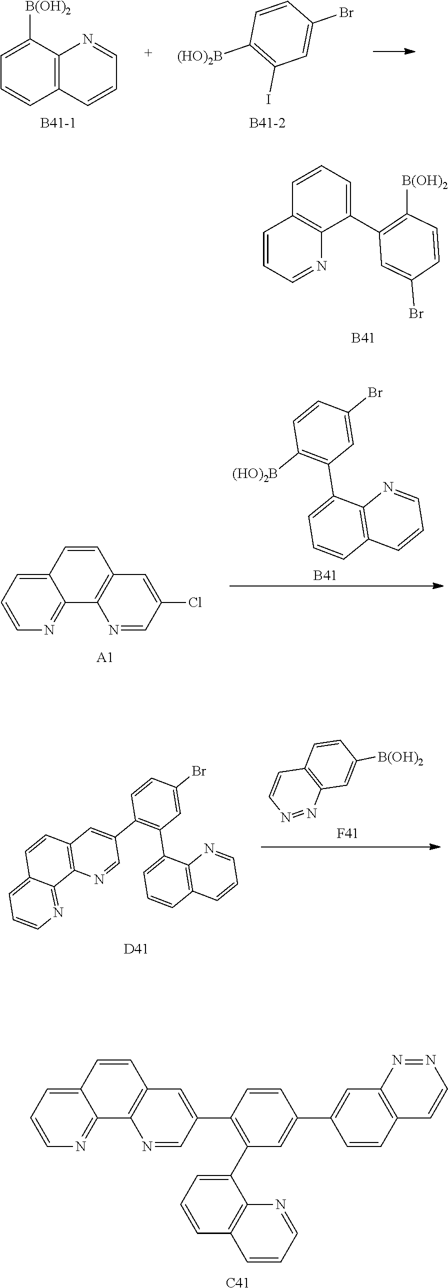

Synthesis of Reactant B41: Nitrogen was introduced in a 250 mL three-necked flask, 0.05 mol of raw material B41-1, 100 mL of THF, 0.04 mol of raw material B41-2 and 0.0004 mol of tetrakis(triphenylphosphine)palladium were added and stirred, 0.06 mol of K₂CO₃ aqueous solution (2 M) was added, and the resulting mixture was heated to 75° C. and reacted at reflux for 10 h. The samples were collected and subjected to TLC until the reaction was completed. The reaction was naturally cooled. The reaction product was extracted with 200 mL of dichloromethane, the layers were separated, and the extract liquor was dried with anhydrous sodium sulfate and filtered. The filtrate was rotary evaporated and purified by a silica gel column to give the reactant B18. The elemental analysis results were as follows: molecular formula: C₁₅H₁₁BBrNO₂, Theoretical: C, 54.93; H, 3.38; B, 3.30; Br, 24.36; N, 4.27; O, 9.76; Found: C, 54.95; H, 3.38; B, 3.30; Br, 24.35; N, 4.27; O, 9.76. m/z: Calc: 327.01, Found: 327.53.

The synthesis method of Compound C41 is similar to the synthesis method of Compound C01, except that B1 was replaced with an equal molar amount of B41, wherein the elemental analysis results of the resulting D31 were as follows: molecular formula: C₂₇H₁₆BrN₃, Theoretical: C, 70.14; H, 3.49; Br, 17.28; N, 9.09; Found: C, 70.11; H, 3.50; Br, 17.29; N, 9.09, and m/z: Calc: 461.05, Found: 461.28; and that F1 in the reaction was replaced with an equal molar amount of F41. C41: MALDI-TOF: m/z: Calc for C₃₅H₂₁N₅: 511.18; Found: 511.44.

The elemental analysis results of the compound were as follows: Calc for C₃₅H₂₁N₅ (%): C, 82.17; H, 4.14; N, 13.69; Found: C, 82.18; H, 4.13; N, 13.68.

### EXAMPLE 6 COMPOUND C97

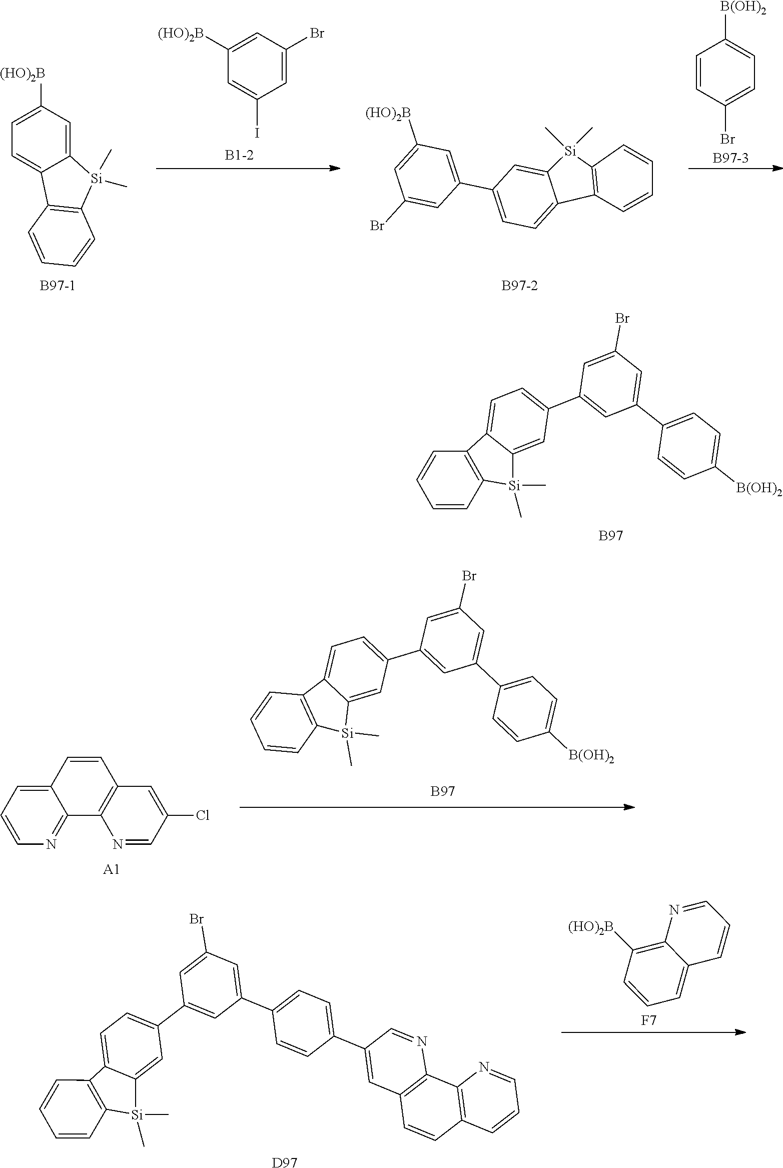

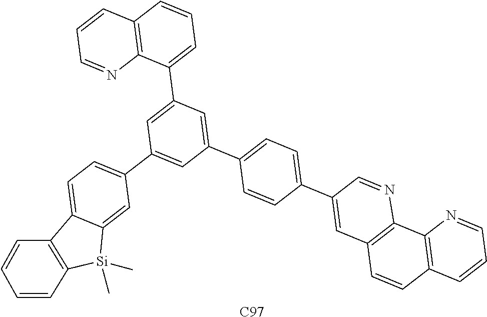 [SMILES: C[Si]1(C)c2ccccc2-c2ccc(-c3cc(-c4ccc(-c5cnc6c(ccc7cccnc76)c5)cc4)cc(-c4cccc5cccnc45)c3)cc21 ; "C97"]

Synthesis of Reactant B97: Nitrogen was introduced in a 250 mL three-necked flask, 0.05 mol of raw material B97-1, 100 mL of THF, 0.04 mol of raw material B1-2 and 0.0004 mol of tetrakis(triphenylphosphine)palladium were added and stirred, 0.06 mol of K₂CO₃ aqueous solution (2 M) was added, and the resulting mixture was heated to 75° C. and reacted at reflux for 10 h. The samples were collected and subjected to TLC until the reaction was completed. The reaction was naturally cooled. The reaction product was extracted with 200 mL of dichloromethane, the layers were separated, and the extract liquor was dried with anhydrous sodium sulfate and filtered. The filtrate was rotary evaporated and purified by a silica gel column to give the reactant B97-2. The elemental analysis results were as follows: molecular formula: C₂₀H₁₈BBrO₂Si,

Theoretical: C, 58.71; H, 4.43; B, 2.64; Br, 19.53; O, 7.82; Si, 6.86; Found: C, 58.70; H, 4.42; B, 2.64; Br, 19.54; O, 7.82; Si, 6.86. m/z: Calc: 408.04, Found: 408.33.

Nitrogen was introduced in a 250 mL three-necked flask, 0.05 mol of reactant

B97-2, 100 mL of THF, 0.04 mol of reactant B97-3 and 0.0004 mol of tetrakis(triphenylphosphine)palladium were added and stirred, 0.06 mol of K₂CO₃ aqueous solution (2 M) was added, and the resulting mixture was heated to 77° C. and reacted at reflux for 10 h. The samples were collected and subjected to TLC until the reaction was completed. The reaction was naturally cooled. The reaction product was extracted with 200 mL of dichloromethane, the layers were separated, and the extract liquor was dried with anhydrous sodium sulfate and filtered. The filtrate was rotary evaporated and purified by a silica gel column to give the reactant B97. The elemental analysis results were as follows: molecular formula: C₂₆H₂₂BBrO₂Si, Theoretical: C, 64.35; H, 4.57; B, 2.23; Br, 16.47; O, 6.59; Si, 5.79; Found: C, 64.37; H, 4.56; B, 2.23; Br, 16.47; O, 6.59; Si, 5.79. m/z: Calc: 484.07, Found: 484.48.

The synthesis method of Compound C97 is similar to the synthesis method of Compound C01, except that B1 was replaced with an equal molar amount of B97, wherein the elemental analysis results of the resulting D97 were as follows: molecular formula: C₃₈H₂₇BrN₂Si, Theoretical: C, 73.66; H, 4.39; Br, 12.90; N, 4.52; Si, 4.53; Found: C, 73.65; H, 4.38; Br, 12.91; N, 4.52; Si, 4.53, and m/z: Calc: 618.11, Found: 618.35; and that F1 in the reaction was replaced with an equal molar amount of F7. C97: MALDI-TOF: m/z: Calc for C47H33N3Si: 667.24; Found: 667.57.

The elemental analysis results of the compound were as follows: Calc for C₄₇H₃₃N₃Si (%): C, 84.52; H, 4.98; N, 6.29; Si, 4.21; Found: C, 84.53; H, 4.99; N, 6.28; Si, 4.21.

### EXAMPLE 7 COMPOUND C111

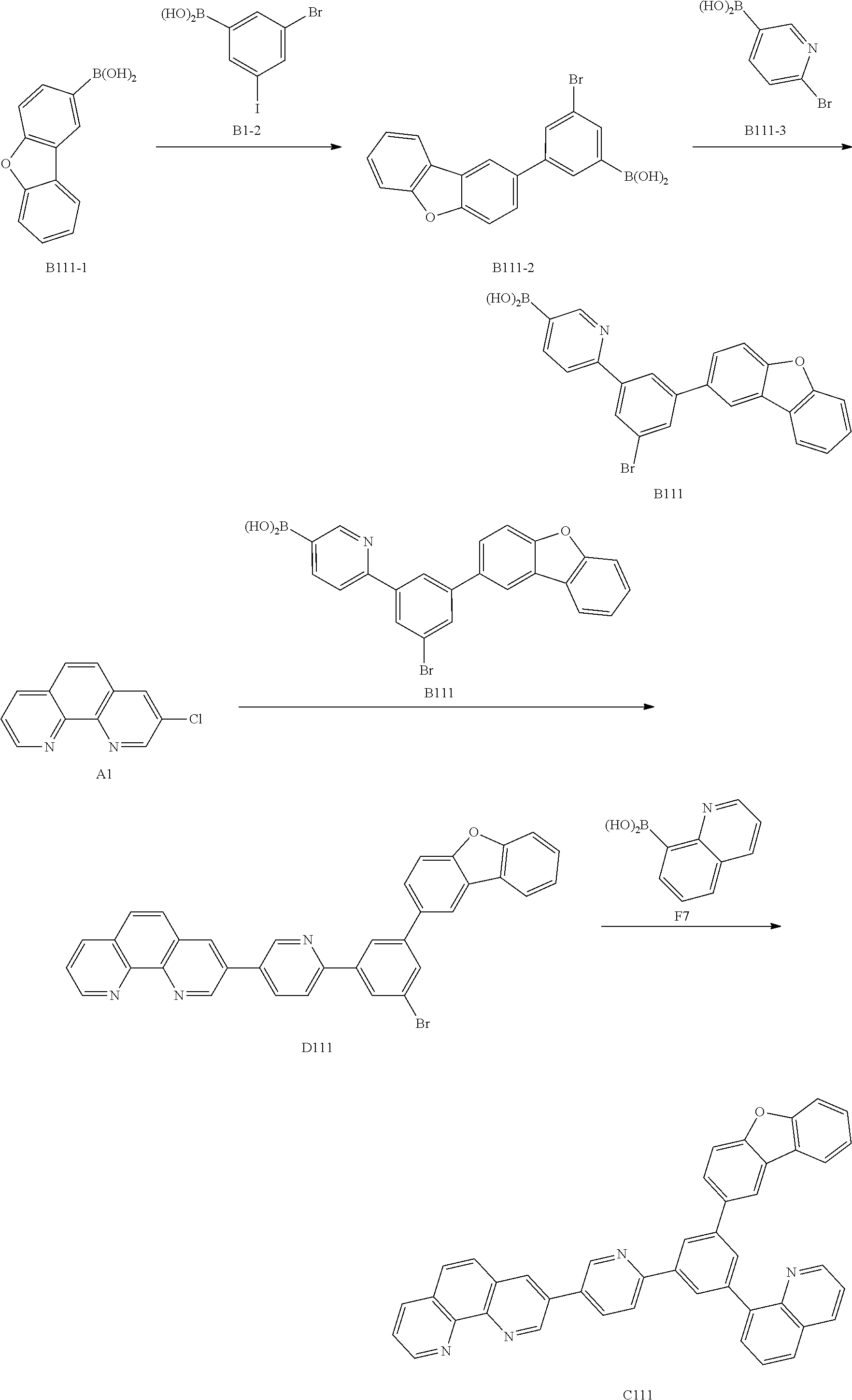

Synthesis of Reactant B111: Nitrogen was introduced in a 250 mL three-necked flask, 0.05 mol of raw material B111-1, 100 mL of THE, 0.04 mol of raw material B1-2 and 0.0004 mol of tetrakis(triphenylphosphine)palladium were added and stirred, 0.06 mol of K₂CO₃ aqueous solution (2 M) was added, and the resulting mixture was heated to 74° C. and reacted at reflux for 10 h. The samples were collected and subjected to TLC until the reaction was completed. The reaction was naturally cooled. The reaction product was extracted with 200 mL of dichloromethane, the layers were separated, and the extract liquor was dried with anhydrous sodium sulfate and filtered. The filtrate was rotary evaporated and purified by a silica gel column to give the reactant B111-2. The elemental analysis results were as follows: molecular formula: C₁₈H₁₂BBrO₃, Theoretical: C, 58.91; H, 3.30; B, 2.95; Br, 21.77; O, 13.08; Found: C, 58.89; H, 3.31; B, 2.95; Br, 21.77; O, 13.08. m/z: Calc: 366.01, Found: 366.37.

Nitrogen was introduced in a 250 mL three-necked flask, 0.05 mol of reactant B111-2, 100 mL of THE, 0.04 mol of reactant B111-3 and 0.0004 mol of tetrakis(triphenylphosphine)palladium were added and stirred, 0.06 mol of K₂CO₃ aqueous solution (2 M) was added, and the resulting mixture was heated to 76° C. and reacted at reflux for 10 h. The samples were collected and subjected to TLC until the reaction was completed. The reaction was naturally cooled. The reaction product was extracted with 200 mL of dichloromethane, the layers were separated, and the extract liquor was dried with anhydrous sodium sulfate and filtered. The filtrate was rotary evaporated and purified by a silica gel column to give the reactant B111. The elemental analysis results were as follows: molecular formula: C₂₃H₁₅BBrNO₃, Theoretical: C, 62.21; H, 3.40; B, 2.43; Br, 17.99; N, 3.15; O, 10.81; Found: C, 62.23; H, 3.40; B, 2.43; Br, 17.98; N, 3.15; O, 10.81. m/z: Calc: 443.03, Found: 443.29.

The synthesis method of Compound C111 is similar to the synthesis method of Compound C01, except that B1 was replaced with an equal molar amount of B111, wherein the elemental analysis results of the resulting D111 were as follows: molecular formula: C35H20BrN3O, Theoretical: C, 72.67; H, 3.48; Br, 13.81; N, 7.26; O, 2.77; Found: C, 72.69; H, 3.47; Br, 13.81; N, 7.26; O, 2.77, and m/z: Calc: 577.08, Found: 577.42; and that F1 in the reaction was replaced with an equal molar amount of F7. C111: MALDI-TOF: m/z: Calc for C₄₄H₂₆N₄₀: 626.21; Found: 626.46.

The elemental analysis results of the compound were as follows: Calc for C₄₄H₂₆N₄₀ (%): C, 84.33; H, 4.18; N, 8.94; O, 2.55; Found: C, 84.35; H, 4.17; N, 8.93; 0, 2.54.

### EXAMPLE 8 COMPOUND C142

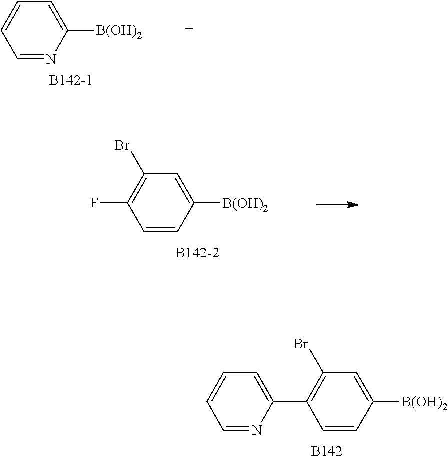 [SMILES: "B142-1" ; "B142-2" ; OB(O)c1ccccn1 ; "B142" ; OB(O)c1ccc(F)c(Br)c1 ; OB(O)c1ccc(-c2ccccn2)c(Br)c1]

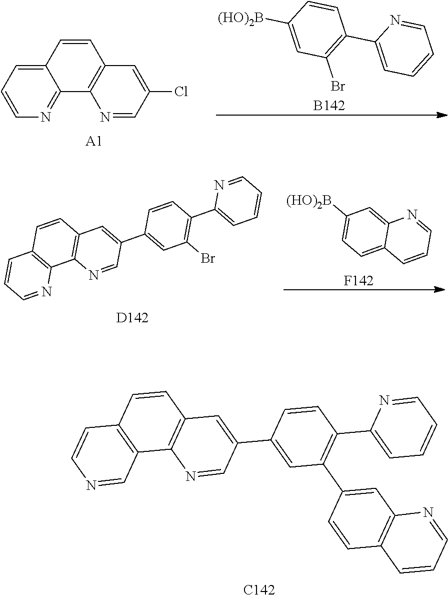

Synthesis of Reactant B142: Nitrogen was introduced in a 250 ml three-necked flask, 0.055 mol of raw material B142-1, 100 mL of THF, 0.04 mol of raw material B142-2, 0.0004 mol of tetrakis(triphenylphosphine)palladium and 0.0003 mol of 1,2-Bis(diphenylphosphino)ethane nickel(II) chloride were added and stirred, 0.06 mol of K₂CO₃ aqueous solution (2 M) was added, and the resulting mixture was heated to 75° C. and reacted at reflux for 10 h. The samples were collected and subjected to TLC until the reaction was completed. The reaction was naturally cooled. The reaction product was extracted with 200 mL of dichloromethane, the layers were separated, and the extract liquor was dried with anhydrous sodium sulfate and filtered. The filtrate was rotary evaporated and purified by a silica gel column to give the reactant B142. The elemental analysis results were as follows: molecular formula: C₁₁H₉BBrNO₂, Theoretical: C, 47.54; H, 3.26; B, 3.89; Br, 28.75; N, 5.04; O, 11.51; Found: C, 47.52; H, 3.27; B, 3.89; Br, 28.75; N, 5.04; O, 11.51. m/z: Calc: 276.99, Found: 277.36.

The synthesis method of Compound C142 is similar to the synthesis method of Compound C01, except that B1 was replaced with an equal molar amount of B142, wherein the elemental analysis results of the resulting D142 were as follows: molecular formula: C₂₃H₁₄BrN₃, Theoretical: C, 67.00; H, 3.42; Br, 19.38; N, 10.19; Found: C, 67.02; H, 3.41; Br, 19.38; N, 10.19, and m/z: Calc: 411.04, Found: 411.47; and that F1 in the reaction was replaced with an equal molar amount of F142. C142: MALDI-TOF: m/z: Calc for C₃₂H₂₀N₄: 460.17; Found: 460.39.

The elemental analysis results of the compound were as follows: Calc for C₃₂H₂₀N₄ (%): C, 83.46; H, 4.38; N, 12.17; Found: C, 83.48; H, 4.37; N, 12.16.

### EXAMPLE 9 COMPOUND C157

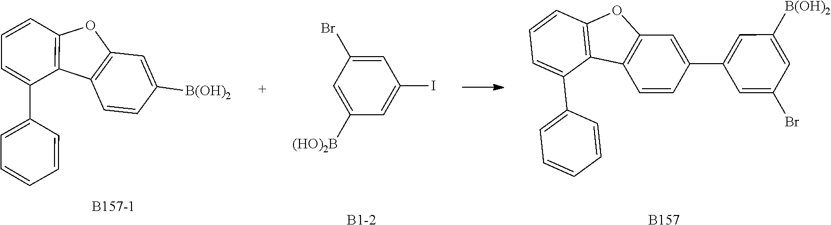 [SMILES: "B157-1" ; OB(O)c1ccc2c(c1)oc1cccc(-c3ccccc3)c12 ; "B157" ; OB(O)c1cc(Br)cc(I)c1 ; "B1-2" ; OB(O)c1cc(Br)cc(-c2ccc3c(c2)oc2cccc(-c4ccccc4)c23)c1]

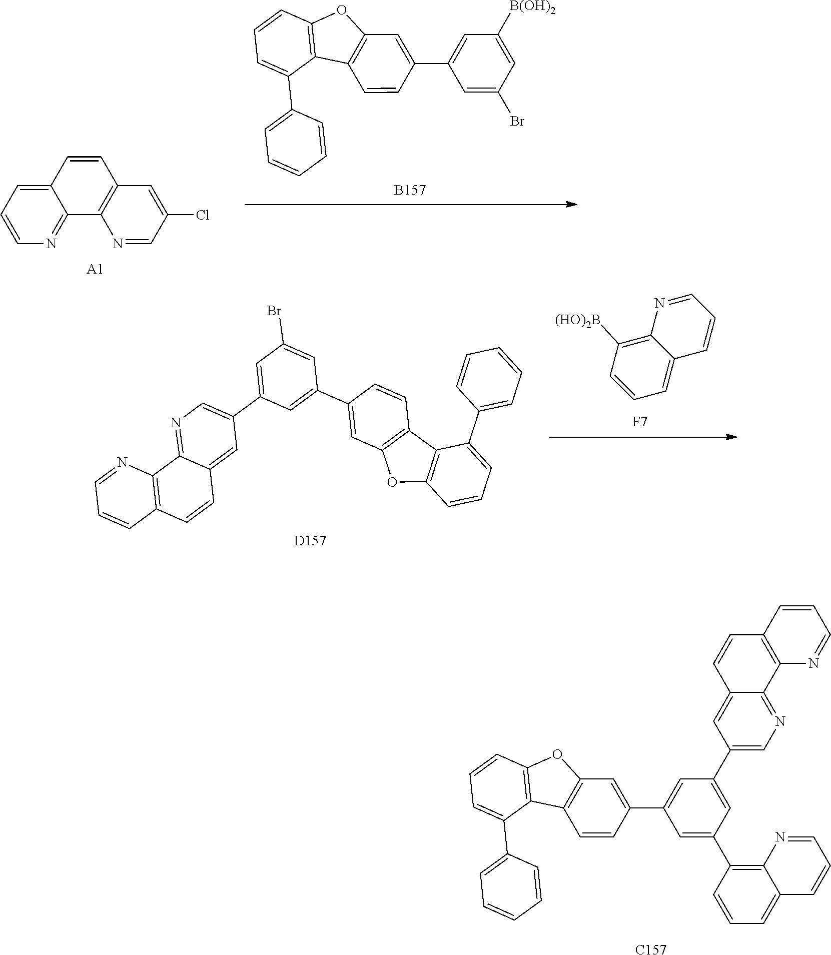

Synthesis of Reactant B157: Nitrogen was introduced in a 250 ml three-necked flask, 0.05 mol of raw material B157-1, 100 mL of THF, 0.04 mol of raw material B1-2 and 0.0004 mol of tetrakis(triphenylphosphine)palladium were added and stirred, 0.06 mol of K₂CO₃ aqueous solution (2 M) was added, and the resulting mixture was heated to 75° C. and reacted at reflux for 10 h. The samples were collected and subjected to TLC until the reaction was completed. The reaction was naturally cooled. The reaction product was extracted with 200 mL of dichloromethane, the layers were separated, and the extract liquor was dried with anhydrous sodium sulfate and filtered. The filtrate was rotary evaporated and purified by a silica gel column to give the reactant B157. The elemental analysis results were as follows: molecular formula: C₂₄H₁₆BBrO₃, Theoretical: C, 65.06; H, 3.64; B, 2.44; Br, 18.03; O, 10.83; Found: C, 65.09; H, 3.63; B, 2.44; Br, 18.03; O, 10.82. m/z: Calc: 442.04, Found: 442.37.

The synthesis method of Compound C157 is similar to the synthesis method of Compound C01, except that B1 was replaced with an equal molar amount of B157, wherein the elemental analysis results of the resulting D157 were as follows: molecular formula: C₃₆H₂₁BrN₂O, Theoretical: C, 74.88; H, 3.67; Br, 13.84; N, 4.85; O, 2.77; Found: C, 74.89; H, 3.66; Br, 13.83; N, 4.85; O, 2.77, and m/z: Calc: 576.08, Found: 576.25; and that F1 in the reaction was replaced with an equal molar amount of F7. C157: MALDI-TOF: m/z: Calc for C₄₅H₂₇N₃₀: 625.22; Found: 625.50.

The elemental analysis results of the compound were as follows: Calc for C₄₅H₂₇N₃₀ (%): C, 86.38; H, 4.35; N, 6.72; O, 2.56; Found: C, 86.39; H, 4.33; N, 6.73; O, 2.55.

### EXAMPLE 10 COMPOUND C162

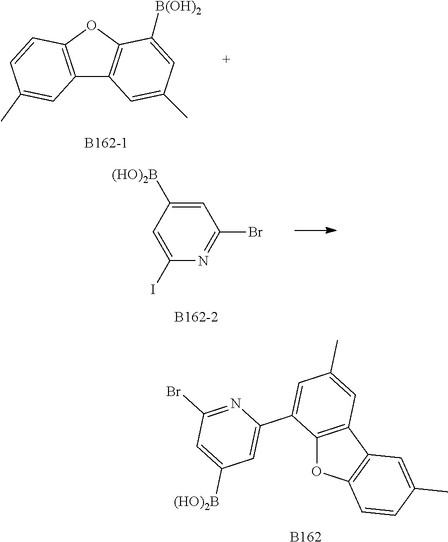

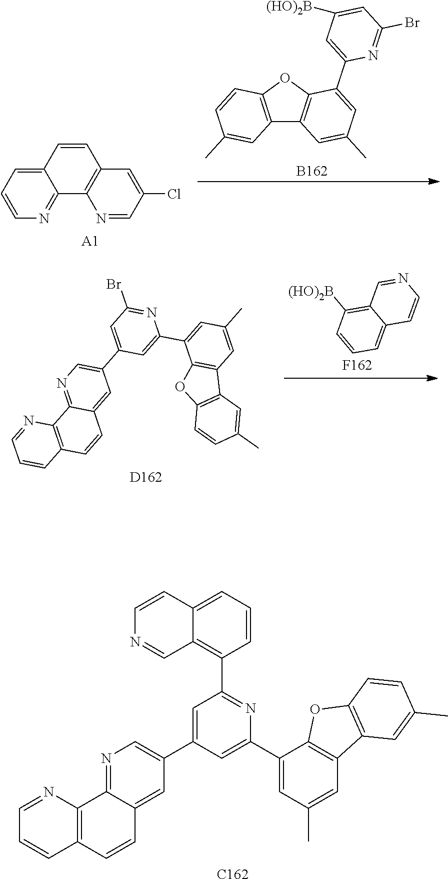

Synthesis of Reactant B162: Nitrogen was introduced in a 250 mL three-necked flask, 0.05 mol of raw material B162-1, 100 mL of THF, 0.04 mol of raw material B162-2 and 0.0004 mol of tetrakis(triphenylphosphine)palladium were added and stirred, 0.06 mol of K₂CO₃ aqueous solution (2 M) was added, and the resulting mixture was heated to 75° C. and reacted at reflux for 10 h. The samples were collected and subjected to TLC until the reaction was completed. The reaction was naturally cooled. The reaction product was extracted with 200 mL of dichloromethane, the layers were separated, and the extract liquor was dried with anhydrous sodium sulfate and filtered. The filtrate was rotary evaporated and purified by a silica gel column to give the reactant B162. The elemental analysis results were as follows: molecular formula: C₁₉H₁₅BBrNO₃, Theoretical: C, 57.62; H, 3.82; B, 2.73; Br, 20.18; N, 3.54; O, 12.12; Found: C, 57.61; H, 3.83; B, 2.73; Br, 20.18; N, 3.54; O, 12.11. m/z: Calc: 395.03, Found: 395.28.

The synthesis method of Compound C162 is similar to the synthesis method of Compound C01, except that B1 was replaced with an equal molar amount of B162, wherein the elemental analysis results of the resulting D162 were as follows: molecular formula: C₃₁H₂₀BrN₃O, Theoretical: C, 70.20; H, 3.80; Br, 15.06; N, 7.92; O, 3.02; Found: C, 70.23; H, 3.79; Br, 15.05; N, 7.92; O, 3.02, and m/z: Calc: 529.08, Found: 529.51; and that F1 in the reaction was replaced with an equal molar amount of F162. C162: MALDI-TOF: m/z: Calc for C₄₀H₂₆N₄O: 578.21; Found: 578.63.

The elemental analysis results of the compound were as follows: Calc for C₄₀H₂₆N₄O (%): C, 83.02; H, 4.53; N, 9.68; O, 2.76; Found: C, 83.03; H, 4.52; N, 9.69; O, 2.76.

EXAMPLE 11 COMPOUND C175

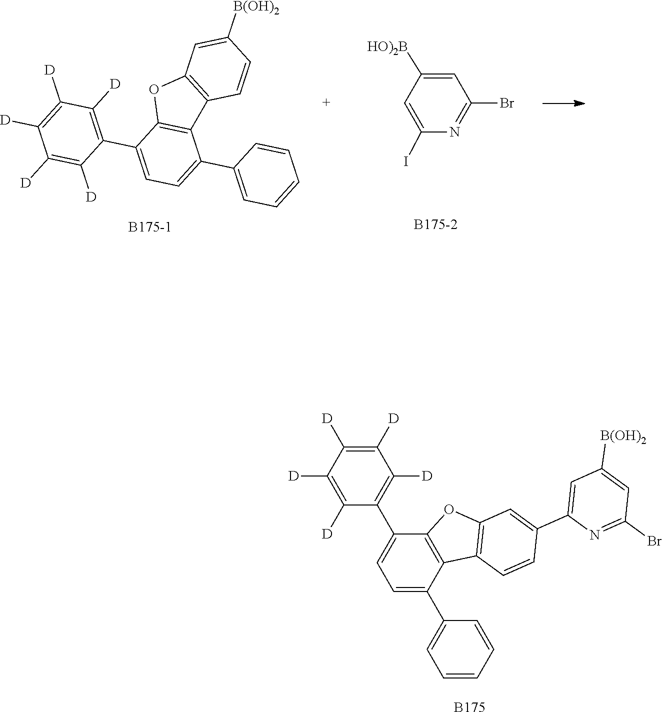

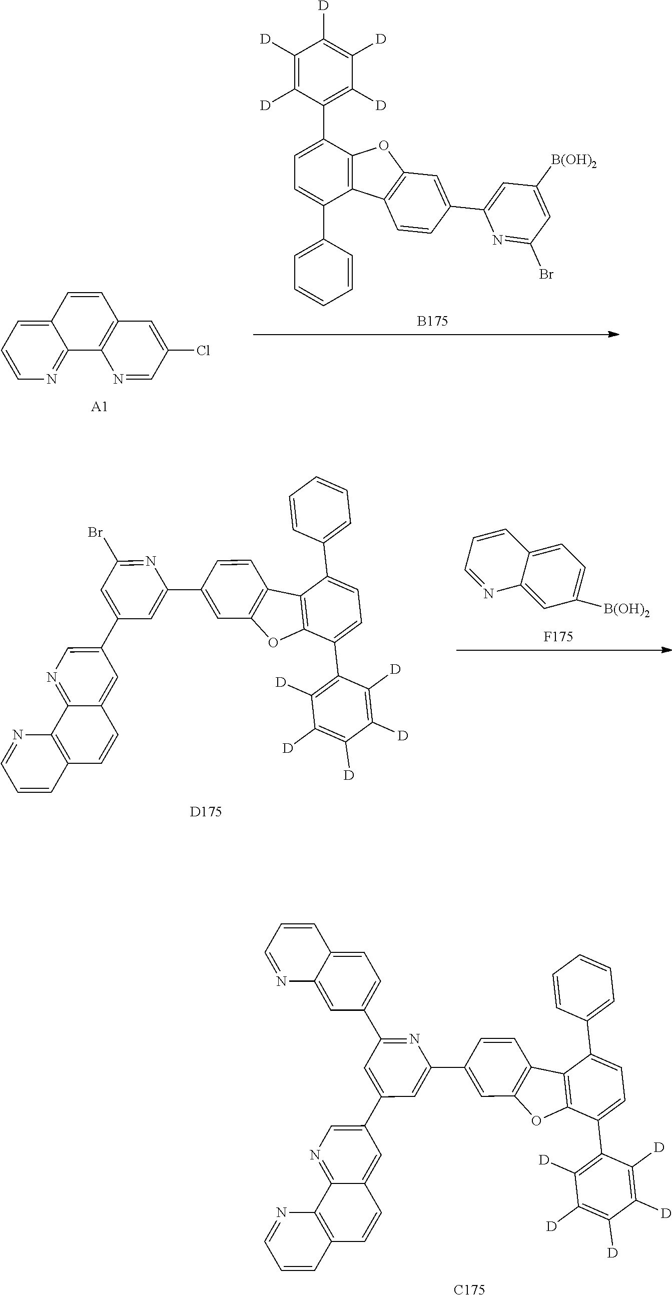

Synthesis of Reactant B175: Nitrogen was introduced in a 250 mL three-necked flask, 0.05 mol of raw material B175-1, 100 mL of THF, 0.04 mol of raw material B175-2 and 0.0004 mol of tetrakis(triphenylphosphine)palladium were added and stirred, 0.06 mol of K₂CO₃ aqueous solution (2 M) was added, and the resulting mixture was heated to 75° C. and reacted at reflux for 10 h. The samples were collected and subjected to TLC until the reaction was completed. The reaction was naturally cooled. The reaction product was extracted with 200 mL of dichloromethane, the layers were separated, and the extract liquor was dried with anhydrous sodium sulfate and filtered. The filtrate was rotary evaporated and purified by a silica gel column to give the reactant B175. The elemental analysis results were as follows: molecular formula: C₂₉H₁₄D₅BBrNO₃, Theoretical: C, 66.32; H, 4.60; B, 2.06; Br, 15.21; N, 2.67; O, 9.14; Found: C, 66.30; H, 4.61; B, 2.06; Br, 15.22; N, 2.67; 0, 9.14. m/z: Calc: 524.10, Found: 524.58.

The synthesis method of Compound C175 is similar to the synthesis method of Compound C01, except that B1 was replaced with an equal molar amount of B175, wherein the elemental analysis results of the resulting D175 were as follows: molecular formula: C₄₁H₁₉D₅BrN₃O, Theoretical: C, 74.66; H, 4.43; Br, 12.11; N, 6.37; O, 2.43; Found: C, 74.64; H, 4.44; Br, 12.10; N, 6.37; O, 2.43, and m/z: Calc: 658.14, Found: 658.50; and that F1 in the reaction was replaced with an equal molar amount of F175. C175: MALDI-TOF: m/z: Calc for C₅₀H₂₅D₅N₄₀: 707.27; Found: 707.55.

The elemental analysis results of the compound were as follows: Calc for C₅₀H₂₅D₅N₄₀ (%): C, 84.84; H, 4.98; N, 7.92; O, 2.26; Found: C, 84.82; H, 4.99; N, 7.92; 0, 2.26.

### EXAMPLE 12 COMPOUND C182

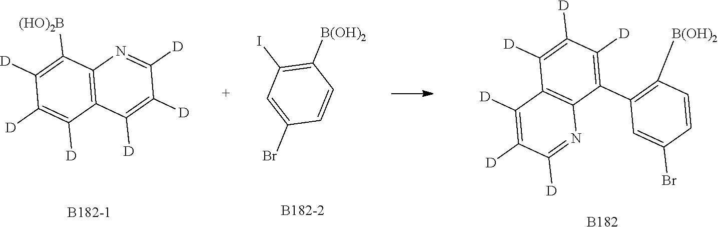 [SMILES: "B182-1" ; "B182-2" ; [2H]c1nc2c(B(O)O)c([2H])c([2H])c([2H])c2c([2H])c1[2H] ; "B182" ; OB(O)c1ccc(Br)cc1I ; [2H]c1nc2c(-c3cc(Br)ccc3B(O)O)c([2H])c([2H])c([2H])c2c([2H])c1[2H]]

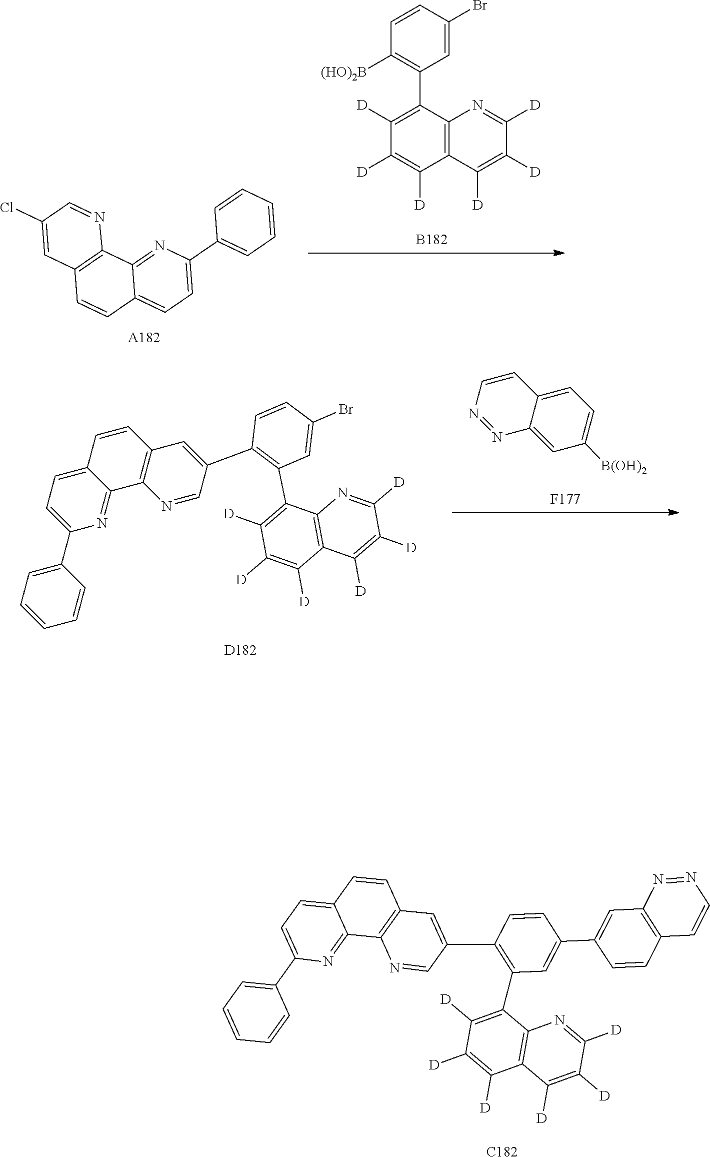

Synthesis of Reactant B182: Nitrogen was introduced in a 250 mL three-necked flask, 0.05 mol of raw material B182-1, 100 mL of THF, 0.04 mol of raw material B182-2 and 0.0004 mol of tetrakis(triphenylphosphine)palladium were added and stirred, 0.06 mol of K₂CO₃ aqueous solution (2 M) was added, and the resulting mixture was heated to 75° C. and reacted at reflux for 10 h. The samples were collected and subjected to TLC until the reaction was completed. The reaction was naturally cooled. The reaction product was extracted with 200 mL of dichloromethane, the layers were separated, and the extract liquor was dried with anhydrous sodium sulfate and filtered. The filtrate was rotary evaporated and purified by a silica gel column to give the reactant B182. The elemental analysis results were as follows: molecular formula: C₁₅H₅D₆BBrNO₂, Theoretical: C, 53.94; H, 5.13; B, 3.24; Br, 23.92; N, 4.19; O, 9.58; Found: C, 53.97; H, 5.12; B, 3.24; Br, 23.91; N, 4.19; O, 9.57. m/z: Calc: 333.04, Found: 333.62.

The synthesis method of Compound C182 is similar to the synthesis method of Compound C01, except that A1 and B1 were replaced with an equal molar amount of A182 and an equal molar amount of B182, respectively, wherein the elemental analysis results of the resulting D182 were as follows: molecular formula: C₃₃H₁₄D₆BrN₃,

Theoretical: C, 72.80; H, 4.81; Br, 14.68; N, 7.72; Found: C, 72.82; H, 4.80; Br, 14.68; N, 7.71, and m/z: Calc: 543.12, Found: 543.49; and that F1 in the reaction was replaced with an equal molar amount of F177. C182: MALDI-TOF: m/z: Calc for C₄₁H₁₉D₆N₅: 593.25; Found: 593.54.

The elemental analysis results of the compound were as follows: Calc for C₄₁H₁₉D₆N₅ (%): C, 82.94; H, 5.26; N, 11.80; Found: C, 82.97; H, 5.25; N, 11.79.

### Device Example 1

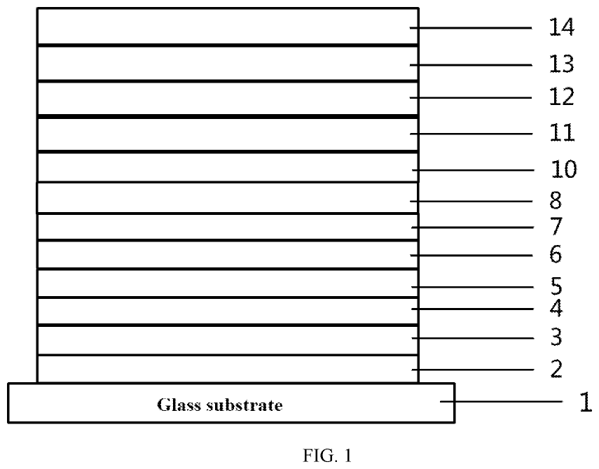

This example provides a tandem OLED device. As shown in FIG. 1, the tandem OLED device includes a substrate 1, an anode 2, a first hole transport layer 3, a second hole transport layer 4, a first light-emitting layer 5, a first electron transport layer 6, an N-type electron generation layer 7, a P-type electron generation layer 8, a third hole transport layer 9, a second light-emitting layer 10, a second electron transport layer 11, a third electron transport layerb 12, a cathode 13, and a capping layer (CPL) 14.

The thickness of the indium tin oxide (ITO) anode is 15 nm, the thickness of the first hole transport layer is 10 nm, the thickness of the second hole transport layer is 20 nm, the thickness of the first light-emitting layer is 45 nm, the thickness of the first electron transport layer is 10 nm, the thickness of the N-type electron generation layer is 20 nm, the thickness of the P-type electron generation layer is 10 nm, the thickness of the third hole transport layer is 40 nm, the thickness of the second light-emitting layer is 45 nm, the thickness of the second electron transport layer is 30 nm, the thickness of the third electron transport layer is 1 nm, the thickness of the cathode is 15 nm, and the thickness of the CPL is 70 nm.

The device was prepared in the following manner.

(1) A glass substrate 1 was cut into a size of 50 mm×50 mm×0.7 mm, sonicated in isopropyl alcohol and deionized water for 30 min separately, and cleaned under ozone for 10 min. The glass substrate having the ITO anode 2 obtained by magnetron sputtering was installed onto a vacuum deposition apparatus.

(2) The compound HAT-CN was deposited by vacuum evaporation on the ITO

anode layer 2 at a vacuum degree of 2×10⁻⁶ Pa as a first hole transport layer 3 with a thickness of 10 nm.

(3) The compound TAPC was deposited by vacuum evaporation on the first hole transport layer 3 as a second hole transport layer 4 with a thickness of 20 nm.

(4) A first light-emitting layer 5 with a thickness of 45 nm was deposited by vacuum evaporation on the second hole transport layer 4 with the organic compound MN as a host material and Ir(piq)₃ as a doped material, wherein the mass ratio of the host material to Ir(piq)₃ was 97:3.

(5) The compound BCP was deposited by vacuum evaporation on the first light-emitting layer as a first electron transport layer 6 with a thickness of 10 nm.

(6) The organic compound C01 provided by the present disclosure was deposited by vacuum evaporation on the first electron transport layer 6 as an N-type electron generation layer 7 with a thickness of 20 nm (the compound C01 was doped with Yb, and the mass ratio of C01 to Yb was 98.5:1.5).

(7) A P-type electron generation layer 8 with a thickness of 10 nm was deposited by vacuum evaporation on the N-type electron generation layer 7 (TAPC was doped with HAT-CN, and the mass ratio of TAPC to HAT-CN was 93:7).

(8) TAPC was deposited by vacuum evaporation on the P-type electron generation layer as a third hole transport layer 9 with a thickness of 40 nm.

(9) A second light-emitting layer 10 with a thickness of 45 nm was deposited by vacuum evaporation on the third hole transport layer 9 with the organic compound MN as a host material and Ir(piq)₃ as a doped material, wherein the mass ratio of the host material to Ir(piq)₃ was 97:3.

(10) The compound BCP was deposited by vacuum evaporation on the second light-emitting layer 10 as a second electron transport layer 11 with a thickness of 30 nm.

(11) Yb was deposited by vacuum evaporation on the second electron transport layer 11 as a third electron transport layer 12 with a thickness of 1 nm.

(12) A magnesium-silver electrode was deposited by vacuum evaporation on the third electron transport layer 12 as a cathode 13 with a thickness of 15 nm, wherein the mass ratio of Mg to Ag was 1:9.

(13) The compound CBP with a high refractive index was deposited by vacuum evaporation on the cathode 13 as a cathode covering layer (that is, a capping layer 14) with a thickness of 70 nm.

The structures of the compounds used in the OLED device are as follows:

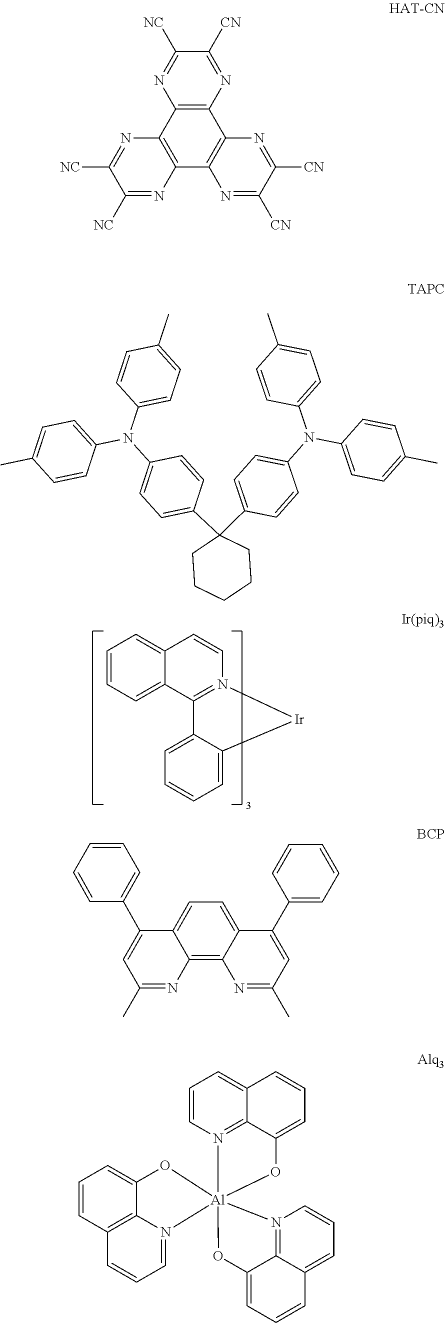

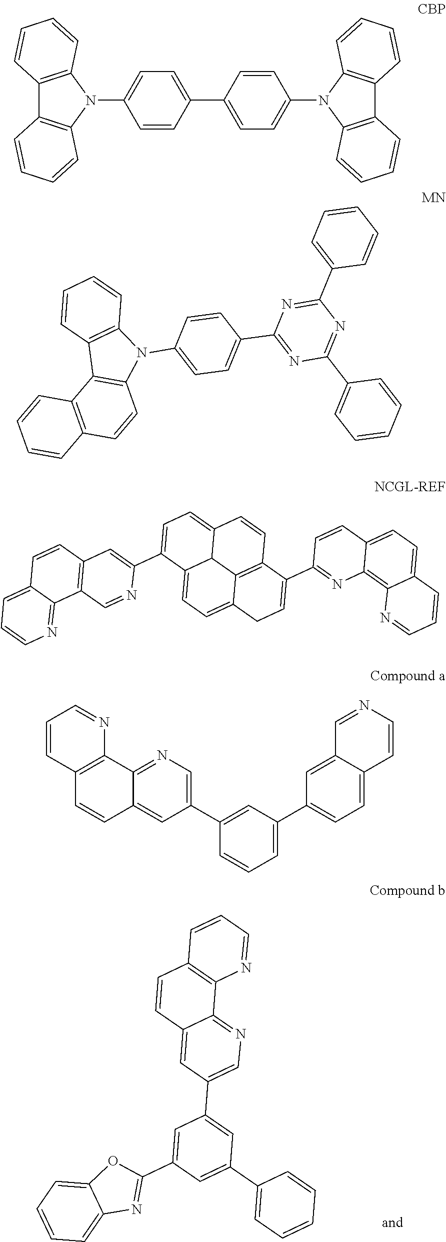

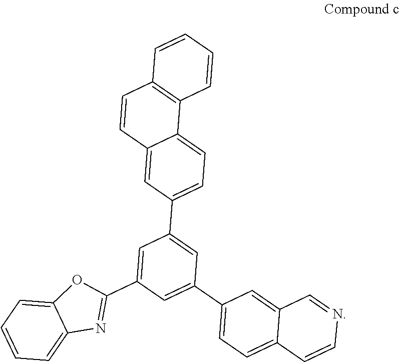 [SMILES: c1ccc2c(c1)ccc1cc(-c3cc(-c4ccc5ccncc5c4)cc(-c4nc5ccccc5o4)c3)ccc12]

### Device Examples 2 to 12

Device Examples 2 to 12 differ from Device Example 1 in that the organic compound C 01 in step (6) in Device Example 1 was replaced with equal amounts of compounds C07, C18, C31, C41, C97, C111, C142, C157, C162, C175, and C182, respectively, and the remaining raw materials and preparation steps are the same.

### Device Comparative Examples 1 to 4

An OLED device was provided here. Device Comparative Examples 1 to 4 differ from Device Example 1 only in that the NCGL material in step (6) was replaced with equal amounts of comparison compounds NCGL-REF, Compound a, Compound b and Compound c, respectively, and the remaining raw materials and preparation steps are the same.

### Performance Evaluation of OLED Devices

The currents of each OLED device at different voltages were tested using a Keithley 2365A digital nanovoltmeter, and then the obtained currents were divided by the luminescence area to obtain the current densities of the OLED device at different voltages. The brightness and radiation energy flux densities of each OLED device at different voltages were tested using a Konicaminolta CS-2000 spectroradiometer. According to the current densities and brightness of each OLED device at different voltages, a working turn-on voltage and current efficiency (Cd/A) at the same current density (10 mA/cm²) were obtained, wherein Vₒₙ denotes the turn-on voltage when the brightness was 1 Cd/m². The lifetime LT95 was obtained by measuring the time taken for each OLED device to reach 95% of its initial brightness.

TABLE 1
Performance evaluation results of OLED devices

| | Light-emitting | Vₒₙ/ | CE/ | LT95/ |
|---|---|---|---|---|
| OLED device | layer material | V_{REF} | CE_{REF} | LT95_{REF} |
| Device Example 1 | C01 | 98.1% | 104.2% | 103.2% |
| Device Example 2 | C07 | 97.9% | 103.9% | 104.5% |
| Device Example 3 | C18 | 97.1% | 105.3% | 104.6% |
| Device Example 4 | C31 | 98.2% | 104.6% | 103.9% |
| Device Example 5 | C41 | 98.5% | 105.3% | 105.1% |
| Device Example 6 | C97 | 97.9% | 104.1% | 104.7% |
| Device Example 7 | C111 | 97.8% | 103.8% | 103.4% |
| Device Example 8 | C142 | 98.0% | 104.7% | 104.7% |
| Device Example 9 | C157 | 97.3% | 104.2% | 103.7% |
| Device Example 10 | C162 | 97.6% | 104.9% | 104.8% |
| Device Example 11 | C175 | 98.2% | 105.5% | 106.2% |
| Device Example 12 | C182 | 98.4% | 105.6% | 106.8% |
| Device Comparative | NCGL-REF | 100% | 100% | 100% |
| Example 1 | | | | |
| Device Comparative | Compound a | 98.2% | 101.5% | 99.8% |
| Example 2 | | | | |
| Device Comparative | Compound b | 101.9% | 99.6% | 101.5% |
| Example 3 | | | | |
| Device Comparative | Compound c | 103.4% | 92.5% | 91.4% |
| Example 4 | | | | |

As can be seen from Table 1, compared with the performance of the organic electroluminescent devices of the device comparative examples, the performance of the organic electroluminescent devices of Examples 1 to 12 is greatly improved. The performance improvement mainly embodies in that the drive voltages of the devices are reduced by about 2%, the efficiency is improved by about 4.7%, and the LT95 lifetimes of the devices are improved by about 4.6%. The improvement may benefit from the particular structure of the compound of the present disclosure. In the present disclosure, the three-dimensional structure of the compound is modified by introducing groups having a larger steric hindrance to enhance the steric nature of the structure of the compound, thereby increasing the singlet energy level of the compound and achieving a high luminescence efficiency. The compound of the present disclosure has excellent thermal stability and thin-film stability, is stable when the OLED works, enables the OLED device prepared with the compound to have a long lifetime, and is a light-emitting material with excellent performance.

The applicant has stated that although the organic compound and the use thereof in the present disclosure are described through the preceding embodiments, the present disclosure is not limited to the preceding embodiments, which means that the implementation of the present disclosure does not necessarily depend on the preceding embodiments. It is to be apparent to those skilled in the art that any improvements made to the present disclosure, equivalent replacements of raw materials of the product, additions of adjuvant ingredients, selections of specific methods, etc. in the present disclosure all fall within the protection scope and the disclosure scope of the present disclosure.

What is claimed is:

1. An organic compound, wherein the organic compound has a structure represented by Formula I: 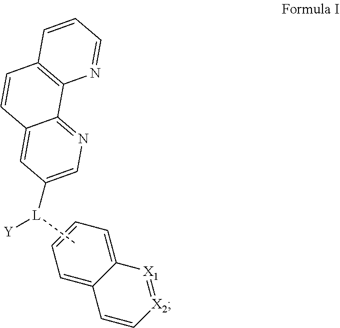
wherein X₁ and X₂ are each independently selected from CR₁ or N; R₁ is selected from hydrogen, a deuterium atom, substituted or unsubstituted C1 to C20 alkyl, substituted or unsubstituted C6 to C30 aryl, substituted or unsubstituted C3 to C20 cycloalkyl, substituted or unsubstituted C1 to C20 alkoxy, substituted or unsubstituted C3 to C30 heterocyclyl or a substituted or unsubstituted C6 to C30 fused ring group;
L is selected from substituted or unsubstituted C6 to C30 aryl or substituted or unsubstituted C3 to C30 heteroaryl; and
Y is selected from substituted or unsubstituted C6 to C30 aryl or substituted or unsubstituted C3 to C30 heteroaryl.

2. The organic compound according to claim 1, wherein a substituent in substituted C6 to C30 aryl or substituted C3 to C30 heteroaryl is selected from at least one of deuterium, cyano, halogen, unsubstituted or halogenated C1 to C10 linear or branched alkyl, unsubstituted or halogenated C1 to C10 alkoxy, C6 to C20 aryl, C2 to C20 heteroaryl or C6 to C18 arylamine.

3. The organic compound according to claim 1, wherein one of X₁ and X₂ is selected from CR₁ and the other is selected from N.

4. The organic compound according to claim 1, wherein X₁ and X₂ are both selected from N.

5. The organic compound according to claim 1, wherein L is selected from phenyl, biphenyl, naphthyl, pyridyl,

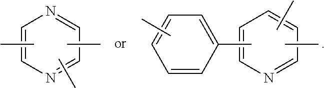

6. The organic compound according to claim 1, wherein L is selected from 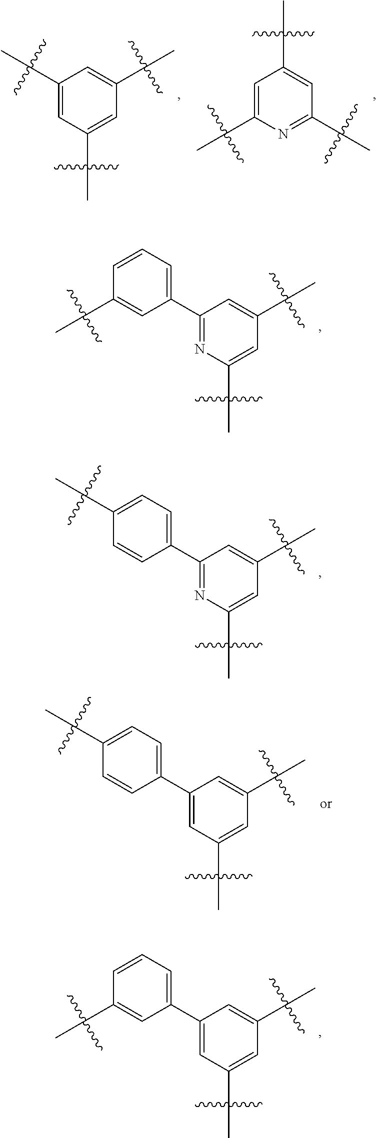 [SMILES: *c1cc(*)cc(*)c1 ; *c1cc(*)nc(*)c1 ; *c1cccc(-c2cc(*)cc(*)n2)c1 ; *c1ccc(-c2cc(*)cc(*)n2)cc1 ; *c1ccc(-c2cc(*)cc(*)c2)cc1 ; *c1cccc(-c2cc(*)cc(*)c2)c1]
wherein the wavy line represents a position of attachment to a group.

7. The organic compound according to claim 1, wherein Y is selected from substituted or unsubstituted phenyl, substituted or unsubstituted naphthyl, substituted or unsubstituted dibenzofuryl, substituted or unsubstituted fluorenyl, spirobifluorenyl, benzodibenzofuryl or substituted or unsubstituted dibenzothienyl.

8. The organic compound according to claim 1, wherein Y is selected from any one of phenyl, biphenyl, naphthyl, anthryl, anthryl-phenyl, phenanthryl, phenanthryl-phenyl, pyrenyl, cyano, carbazolyl, acridinyl, phenanthrolinyl, triarylamino, dibenzothienyl, dibenzofuryl, benzoxazolyl, dimethylfluorenyl, spirobifluorenyl or benzoquinolyl.

9. The organic compound according to claim 1, wherein the organic compound comprises any one of the following compounds: 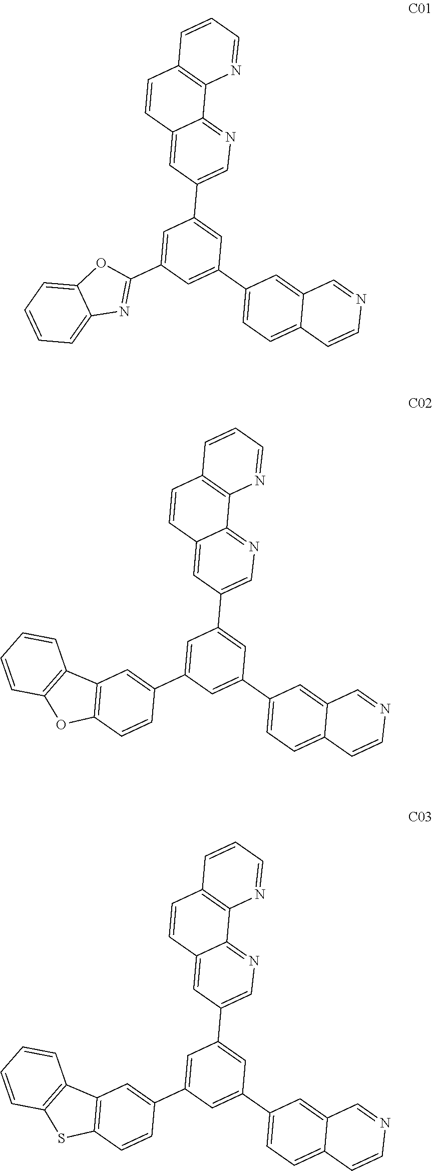 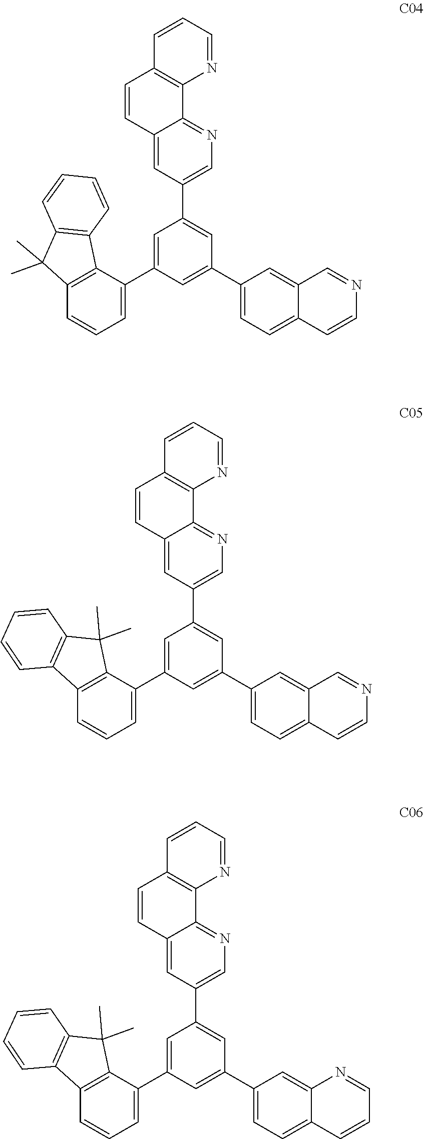 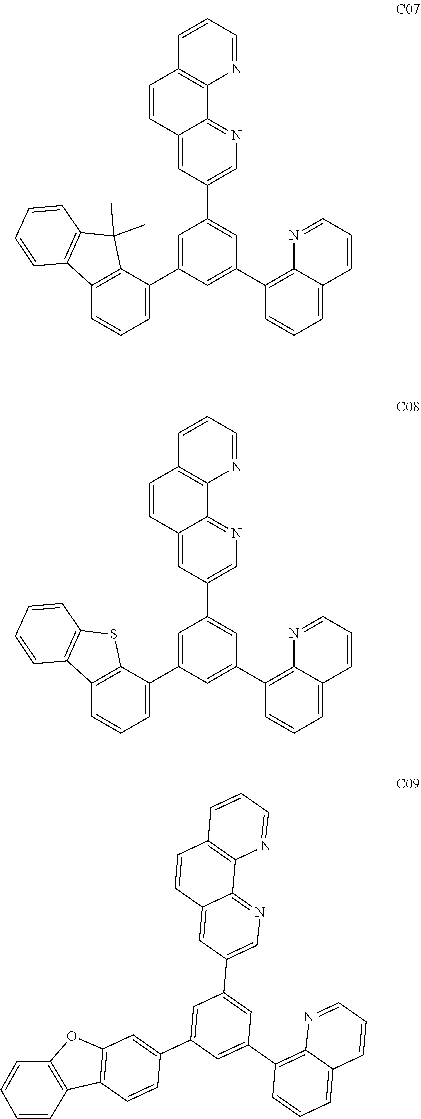 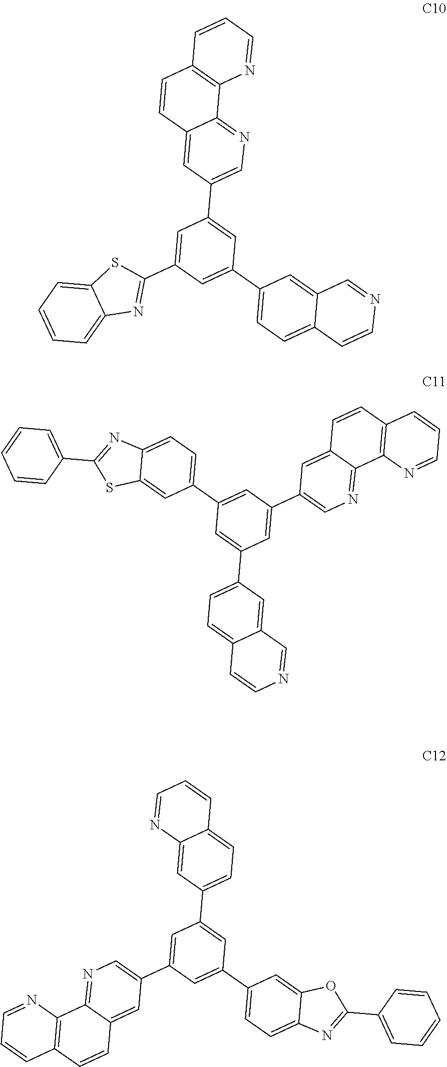 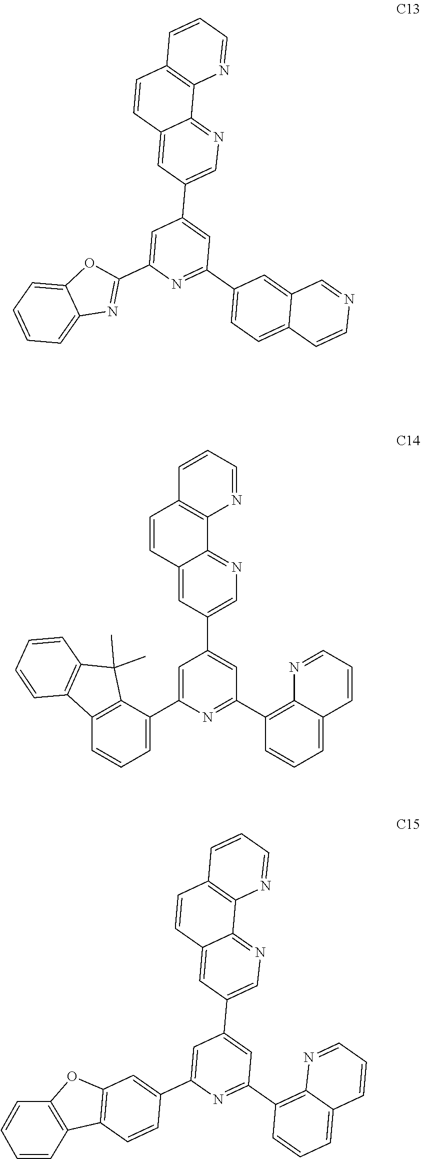 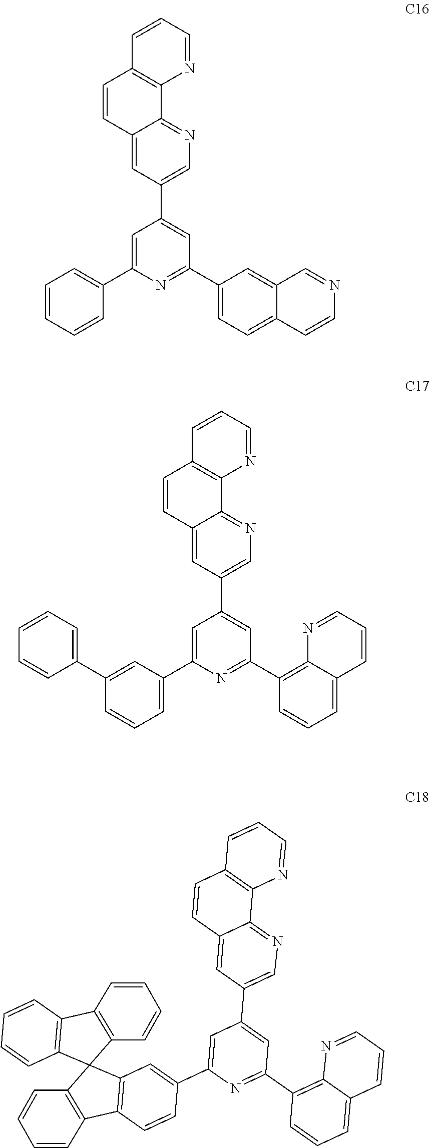 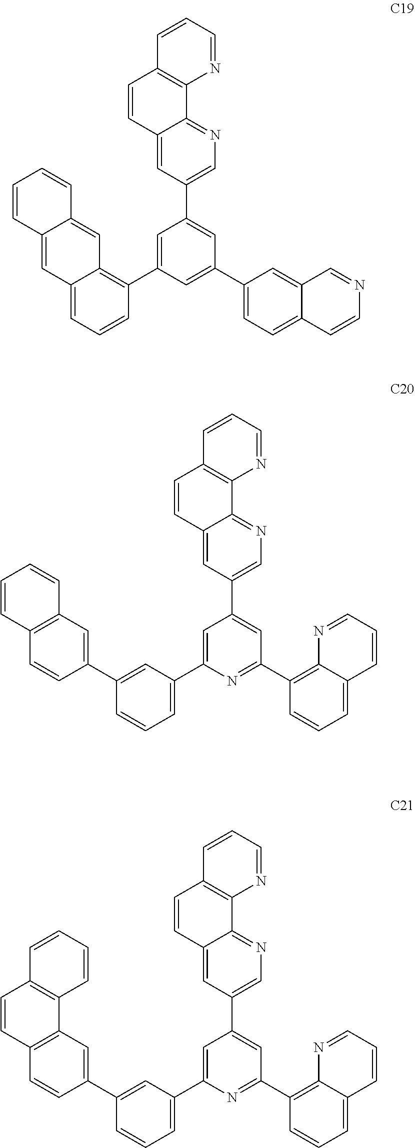 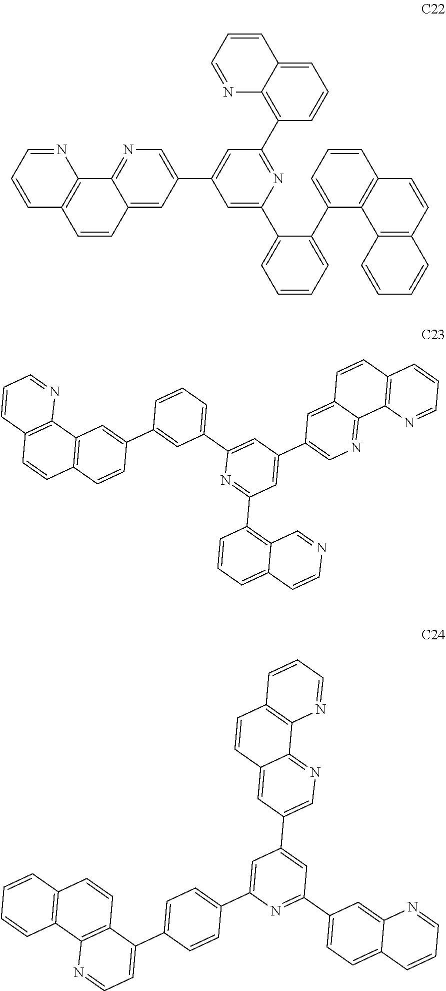 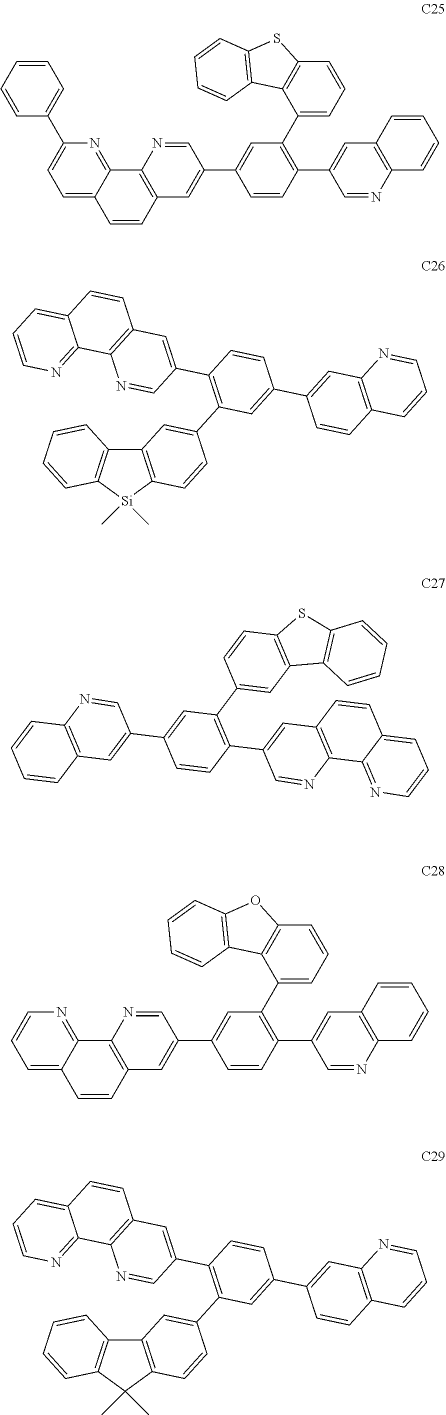 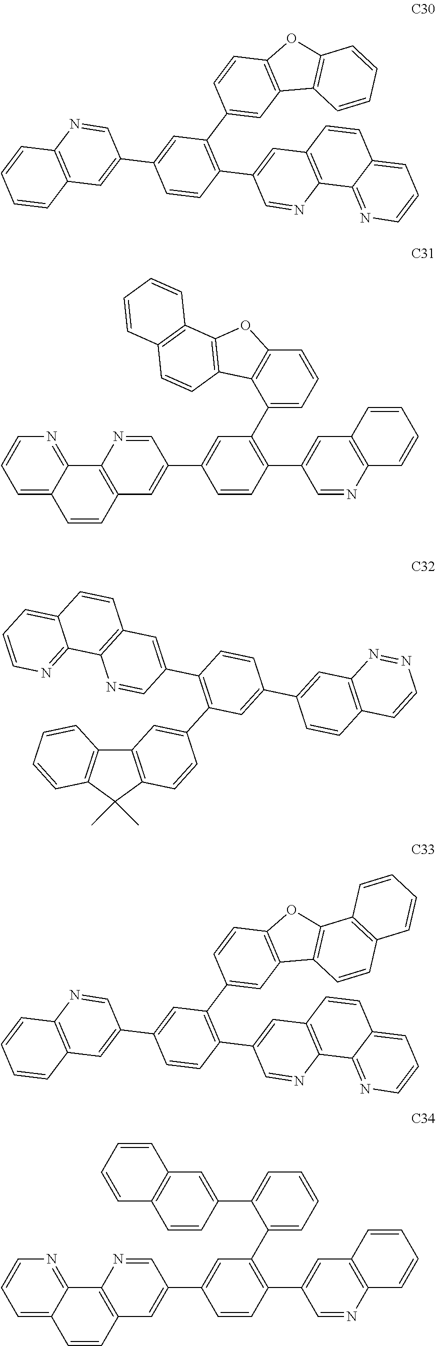 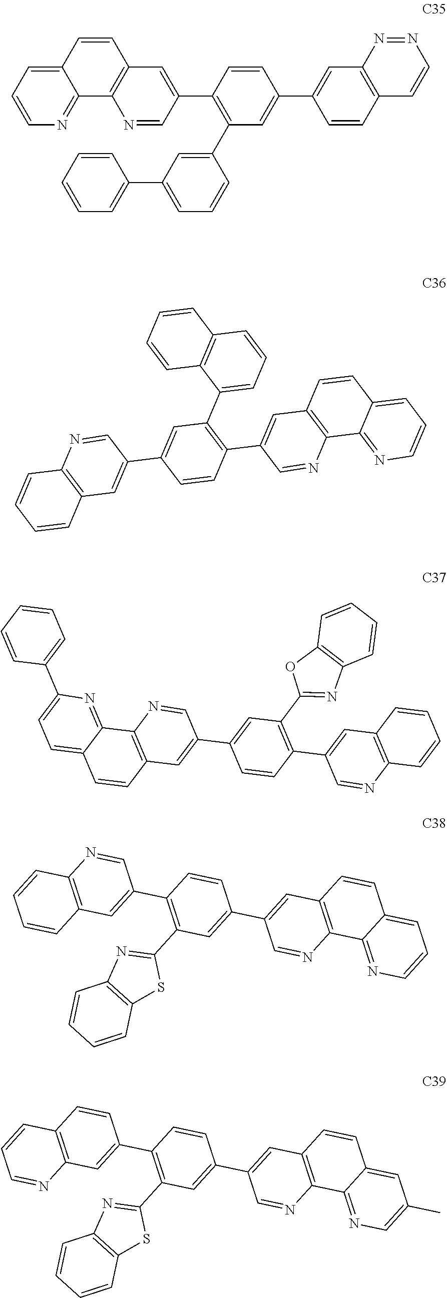 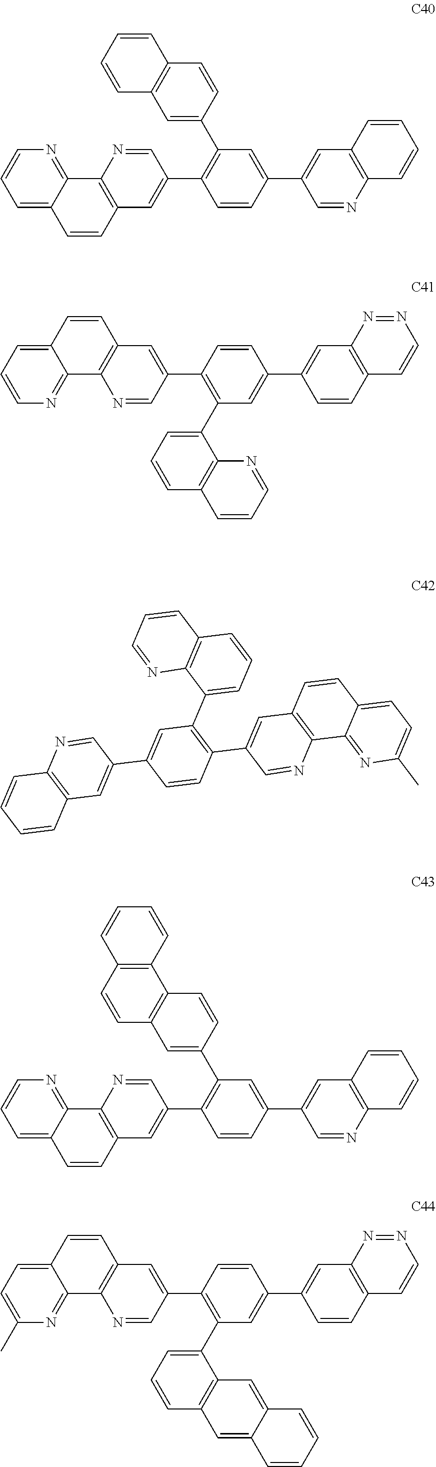 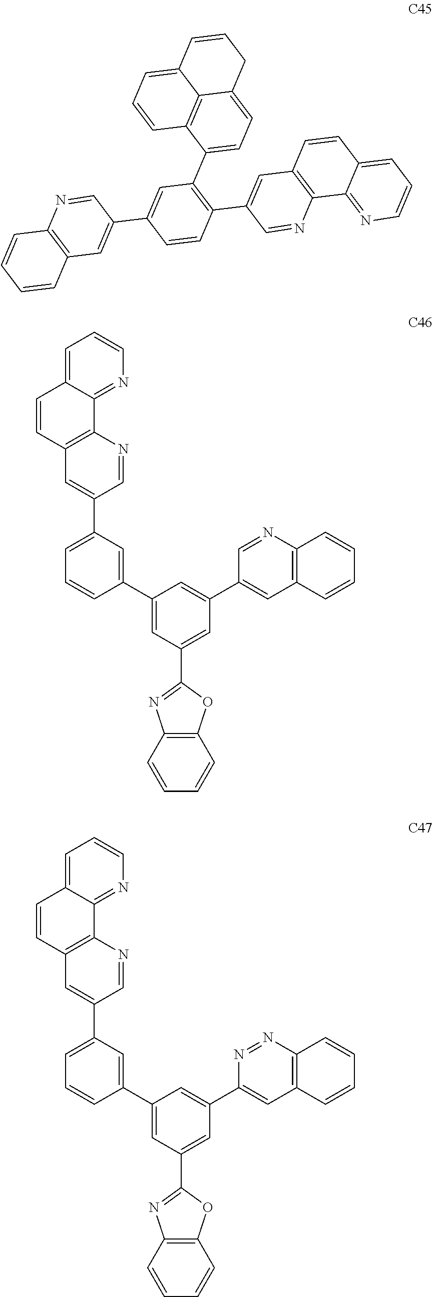 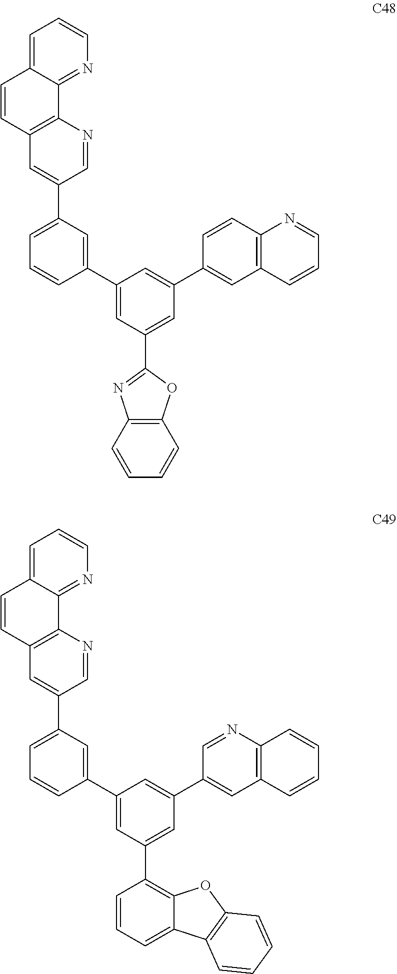 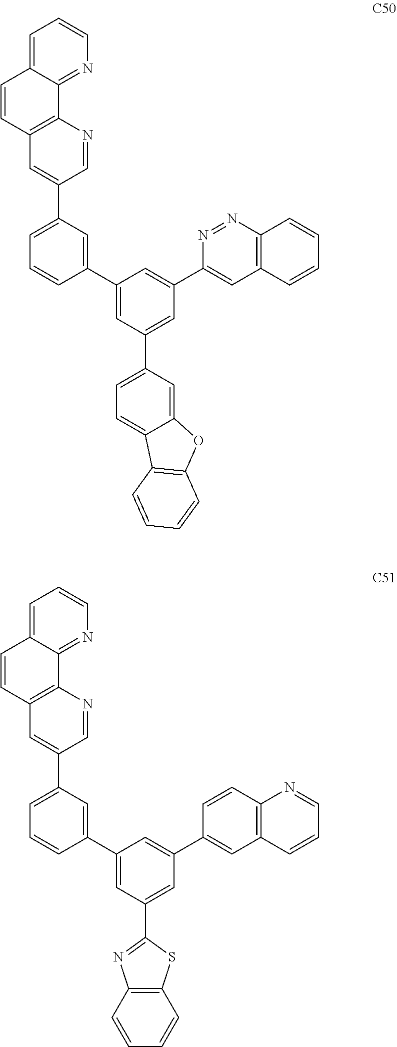 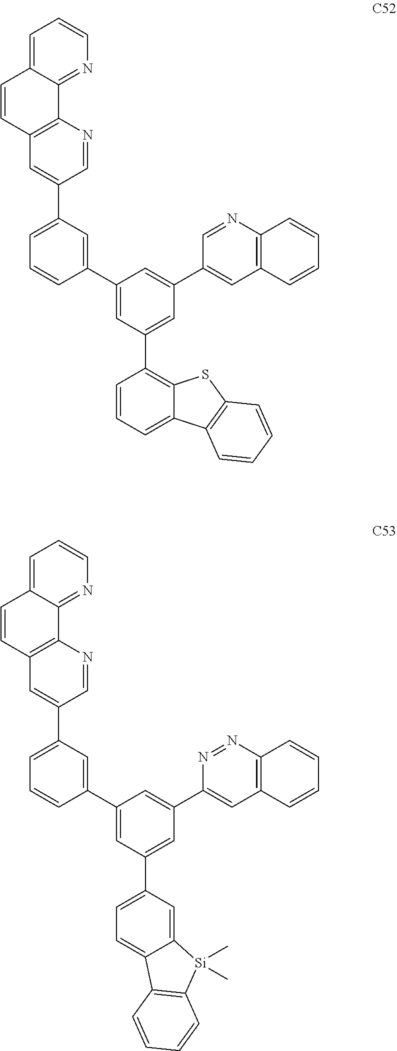 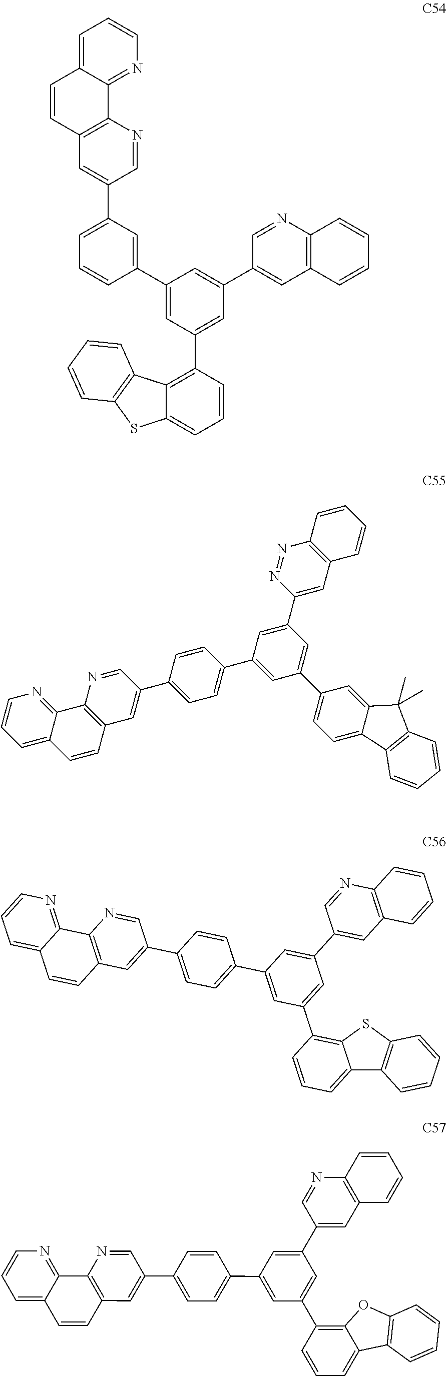 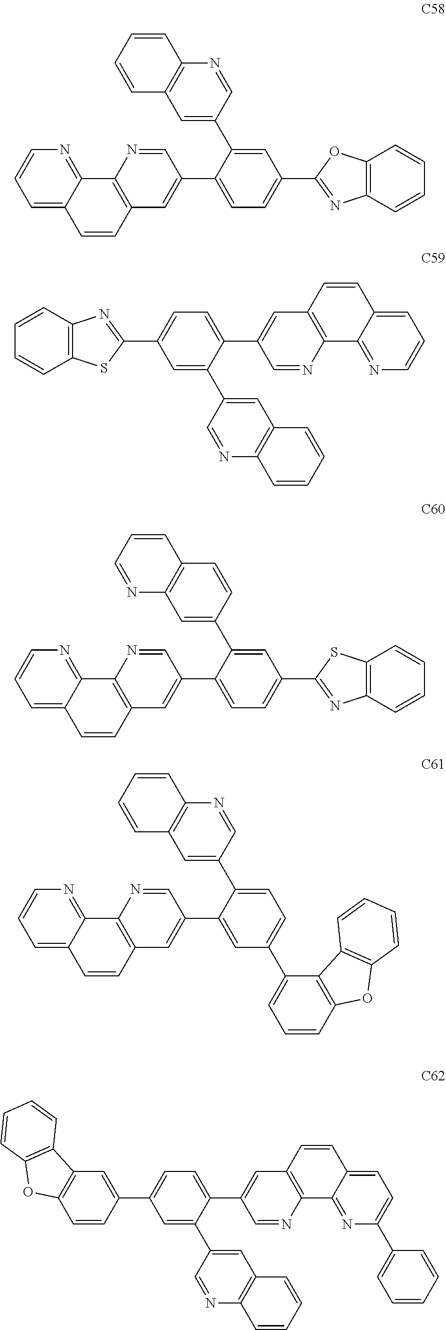 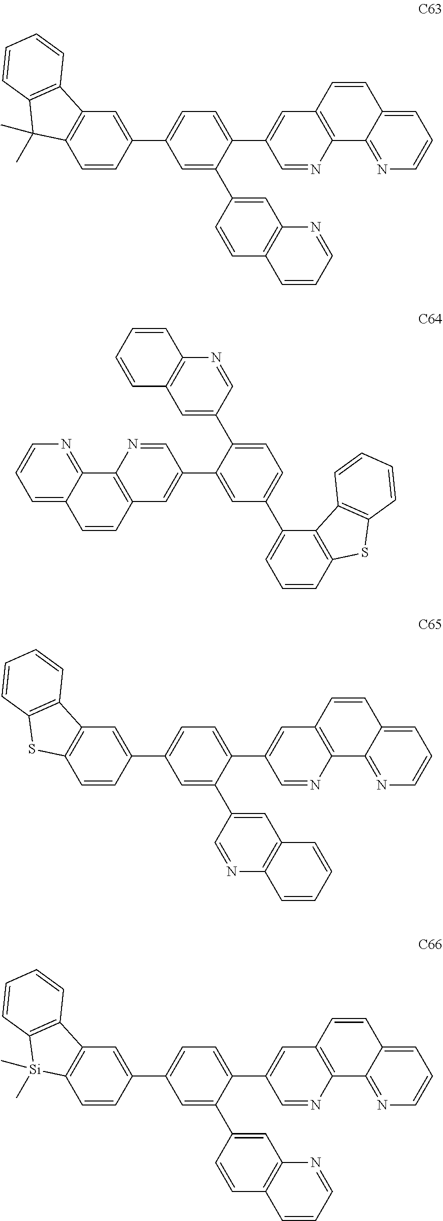 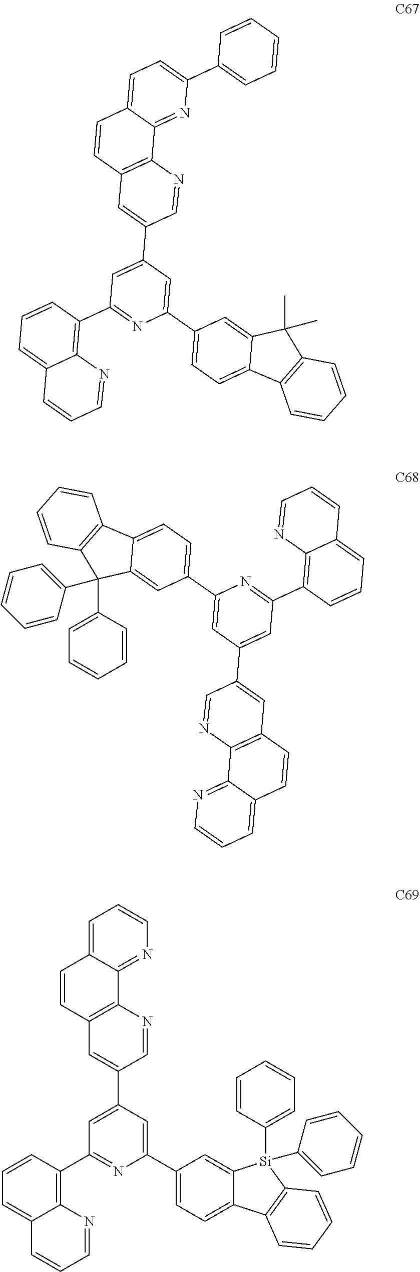 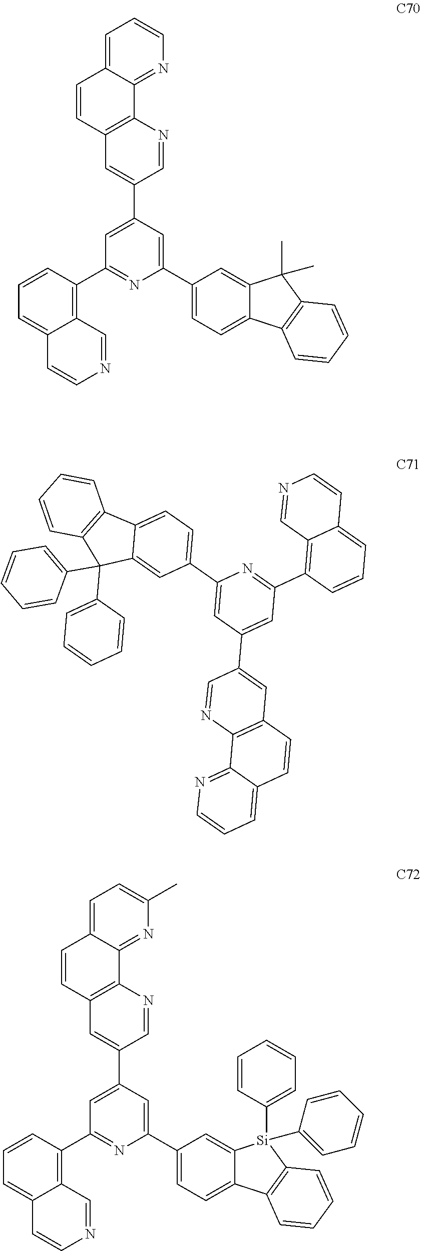 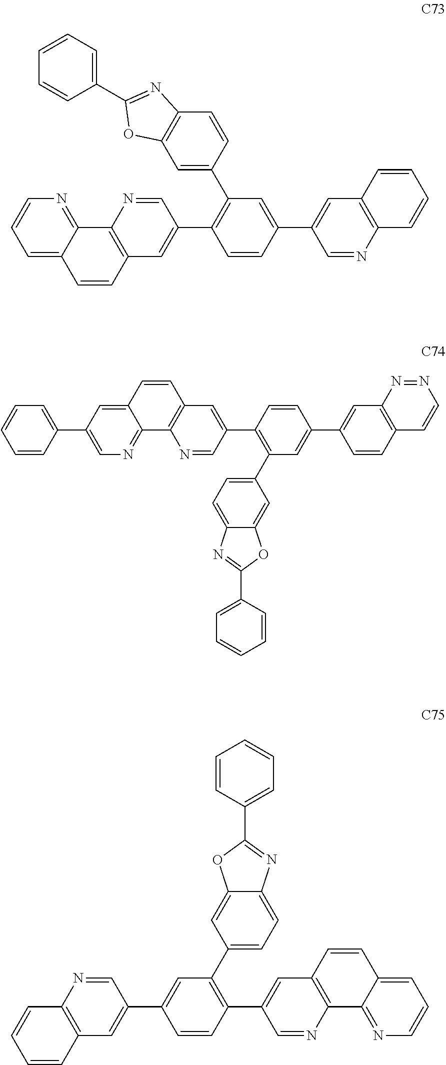 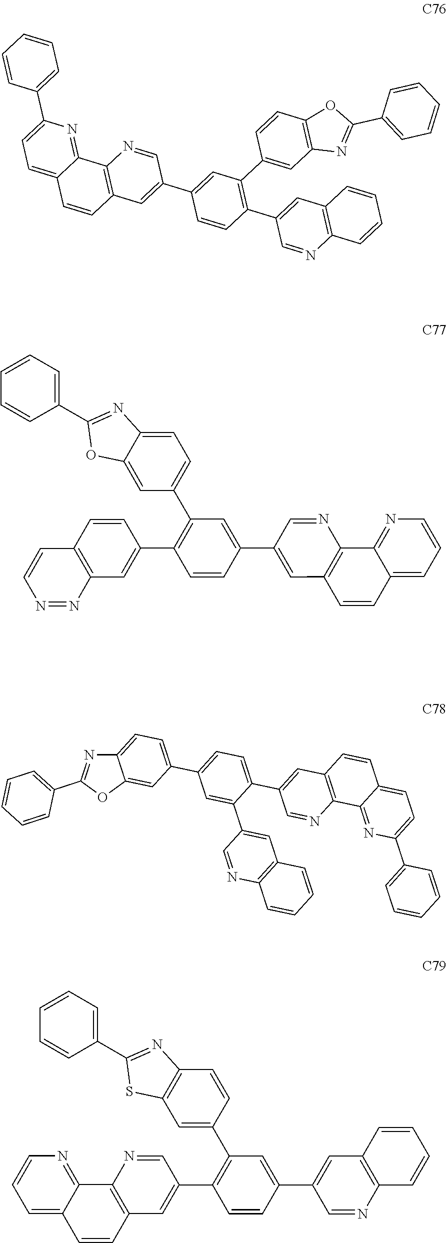 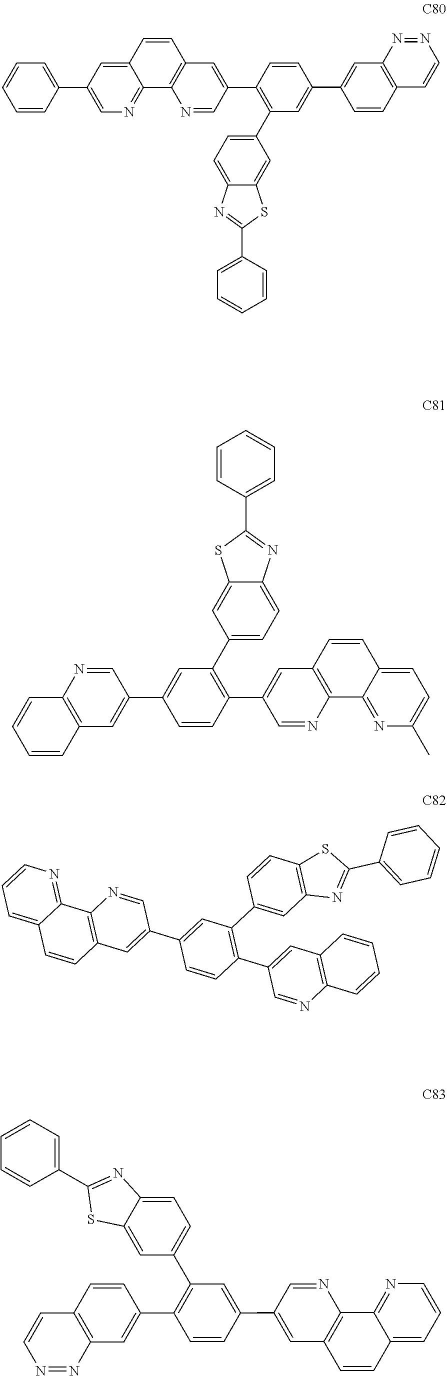 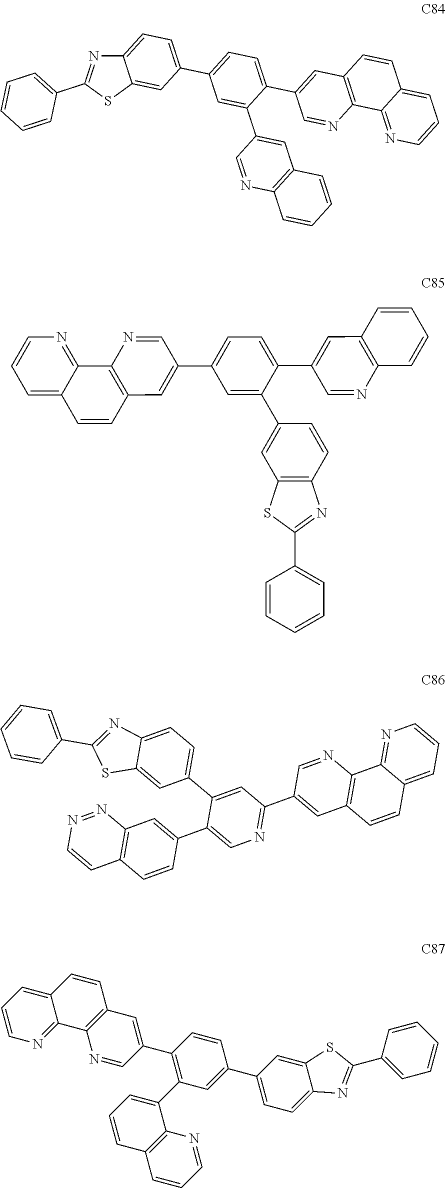 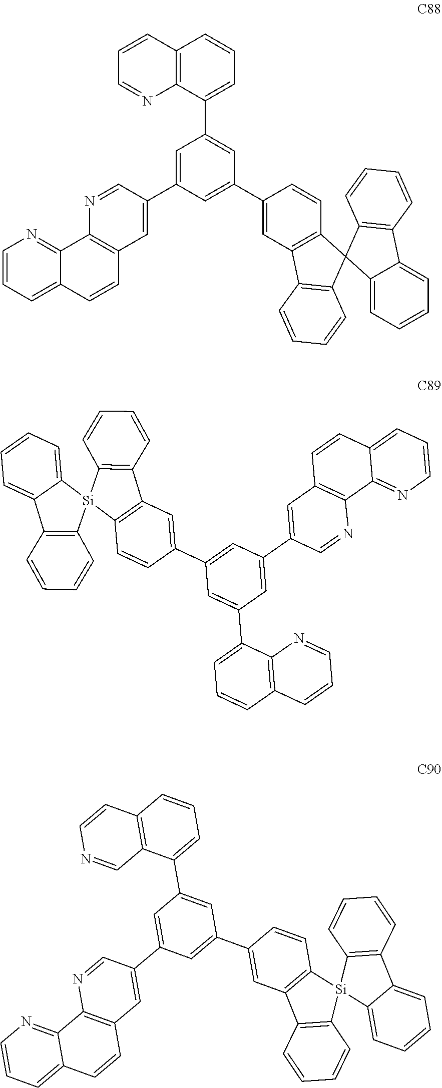 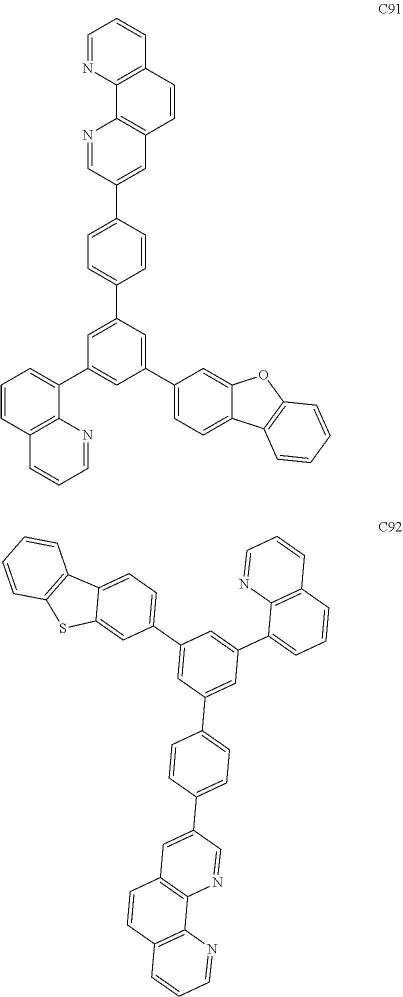 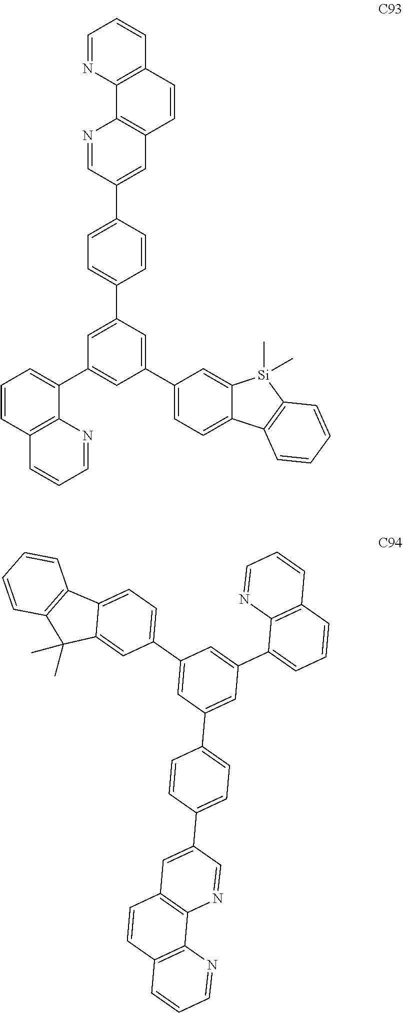 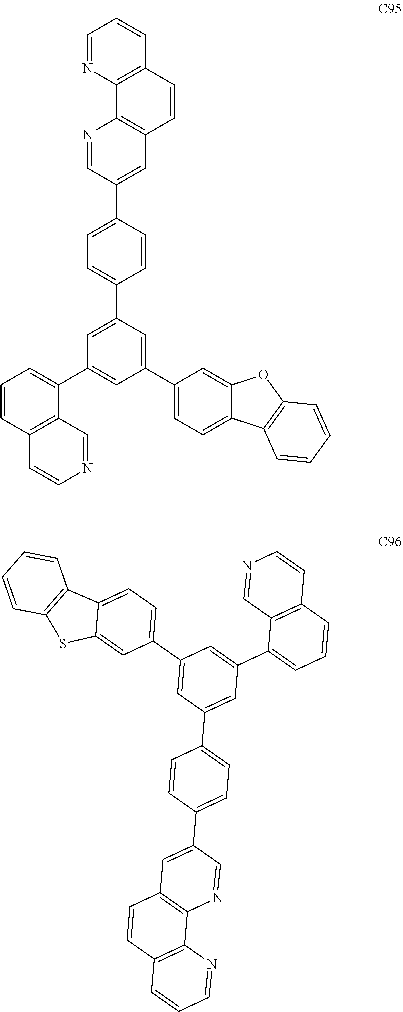 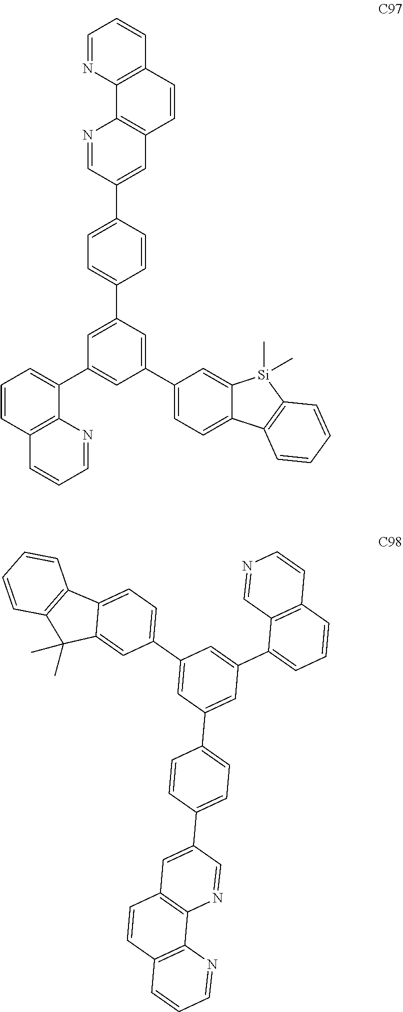 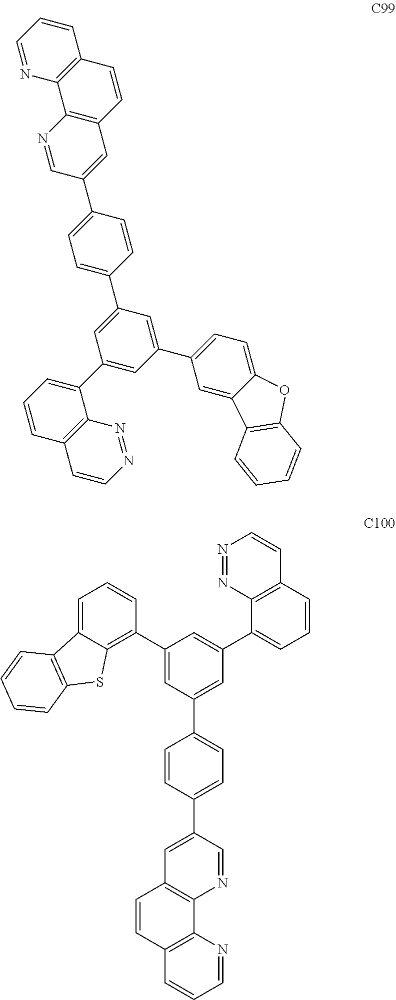 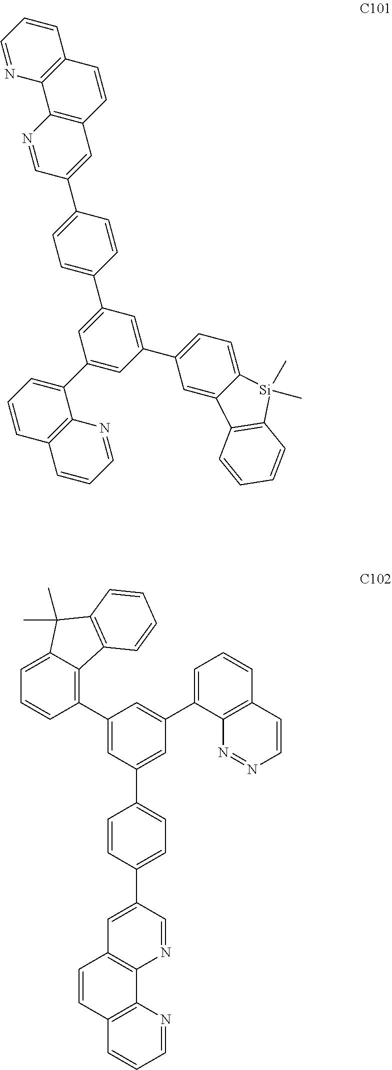 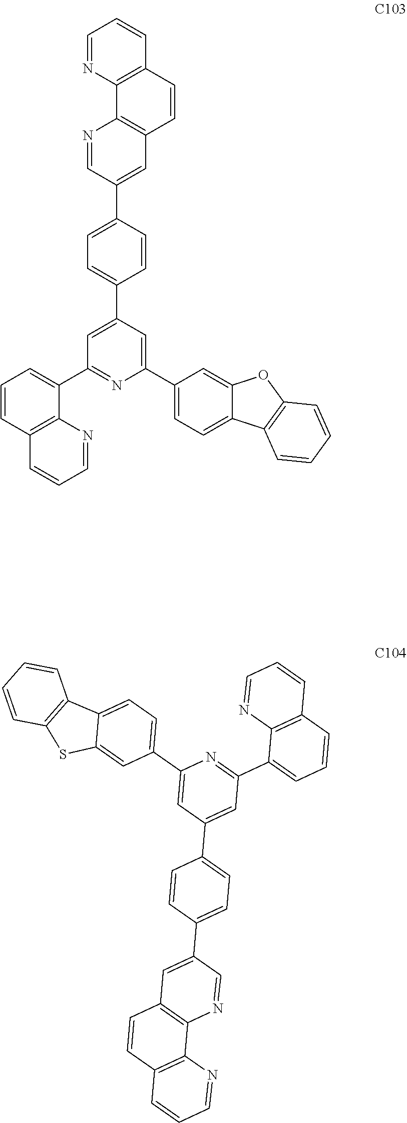 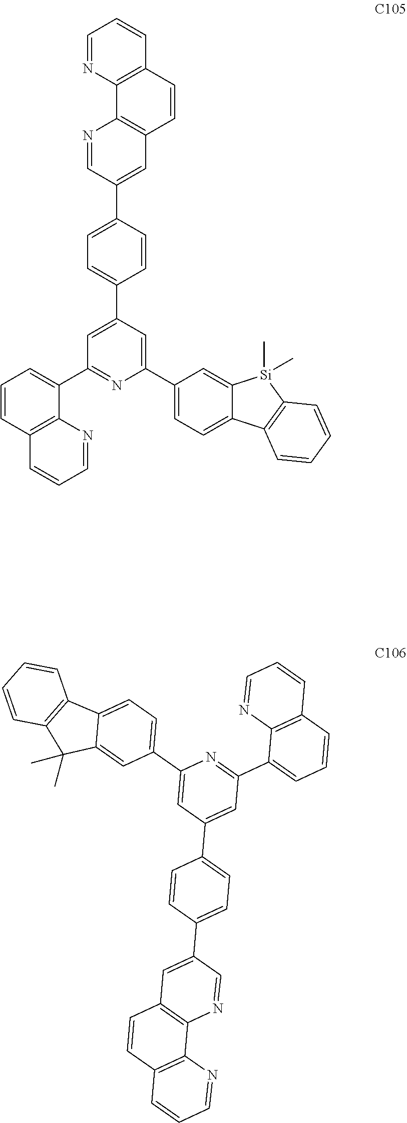 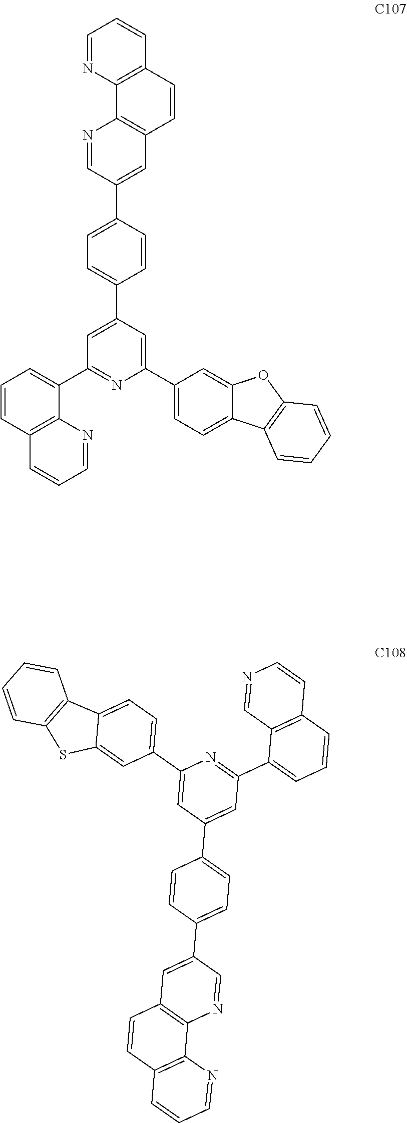 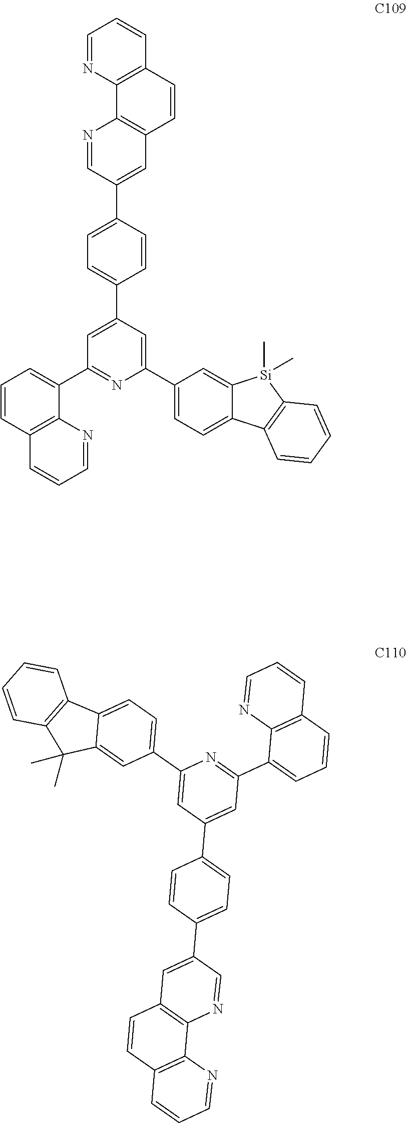 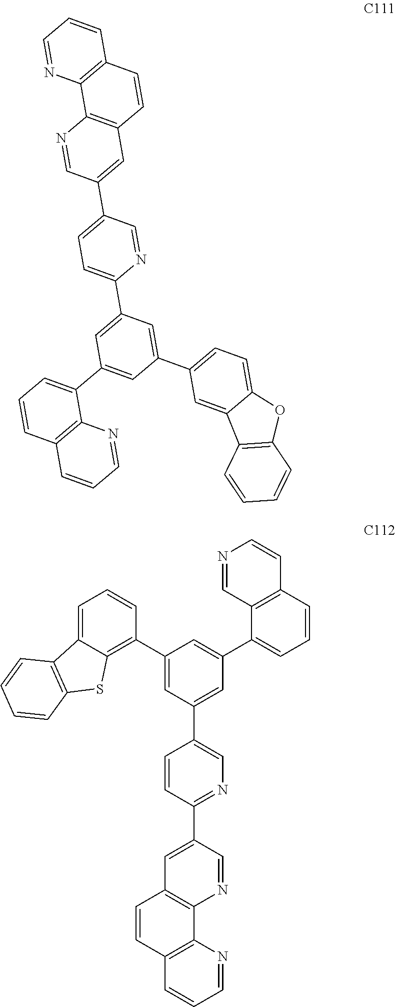 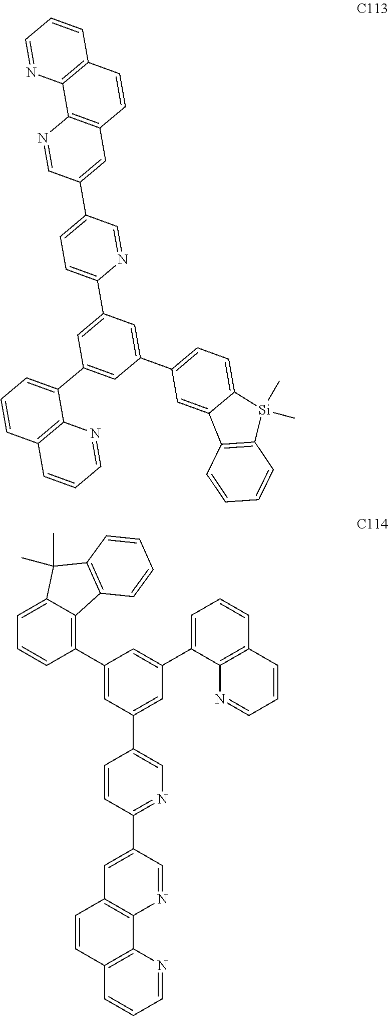 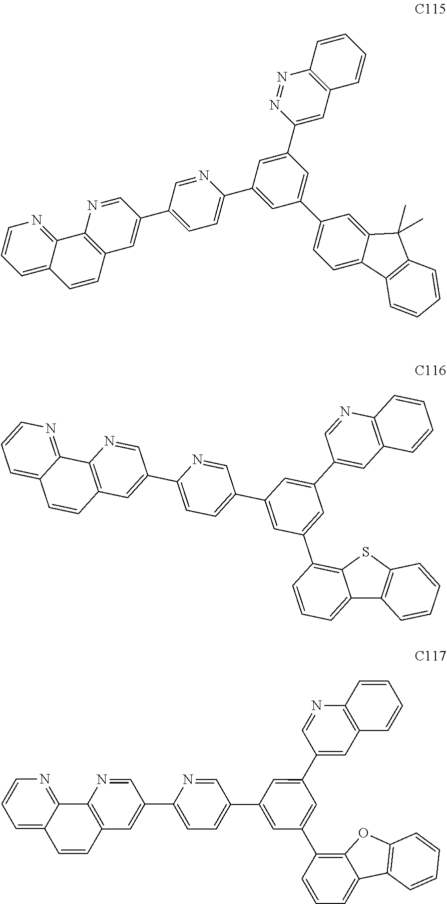 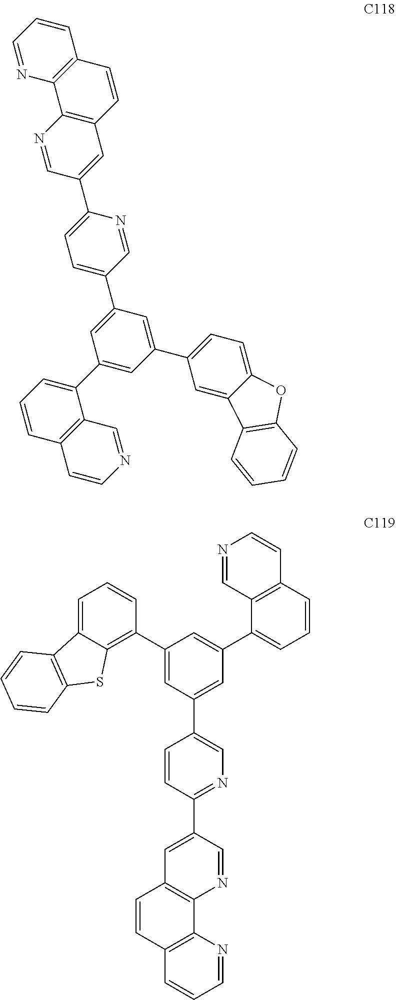 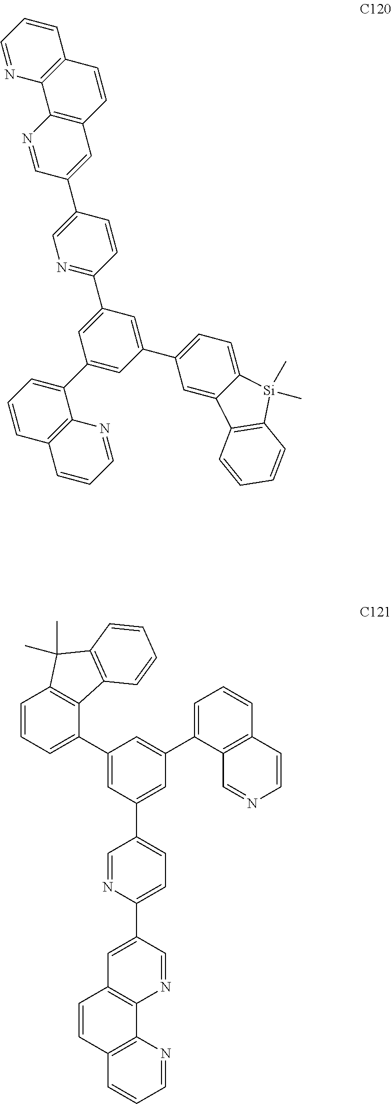 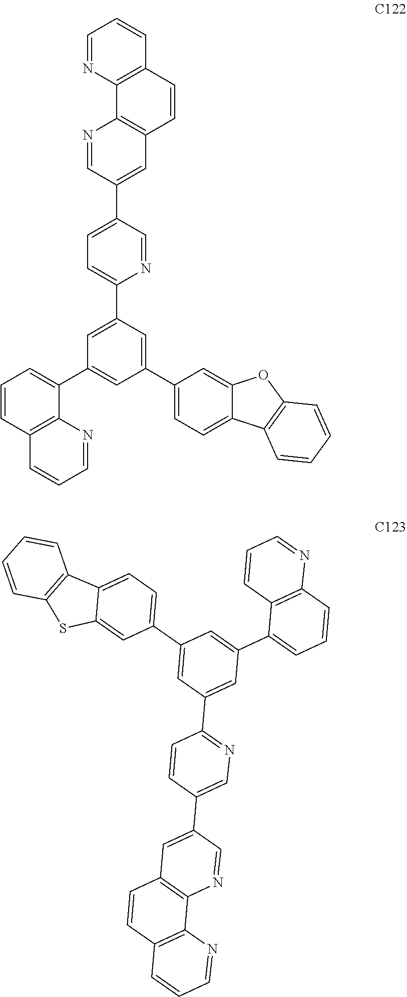 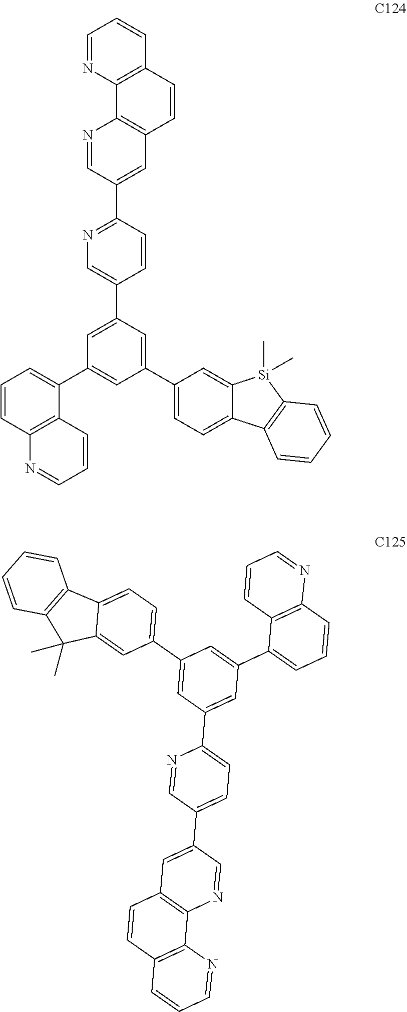 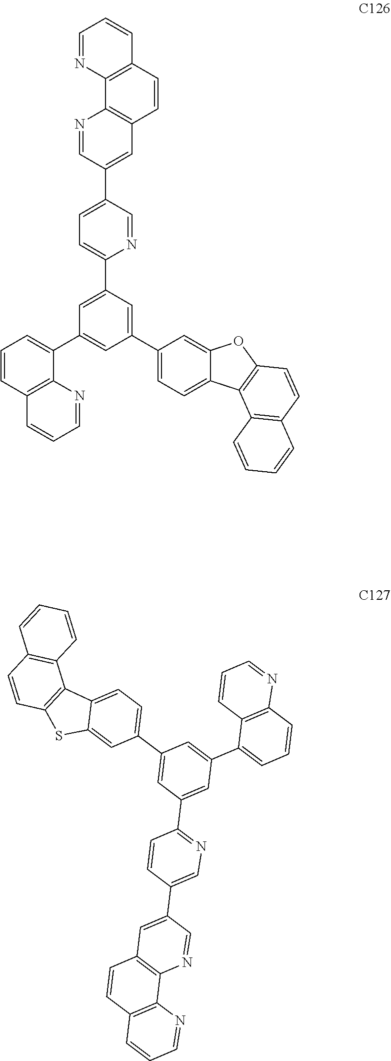 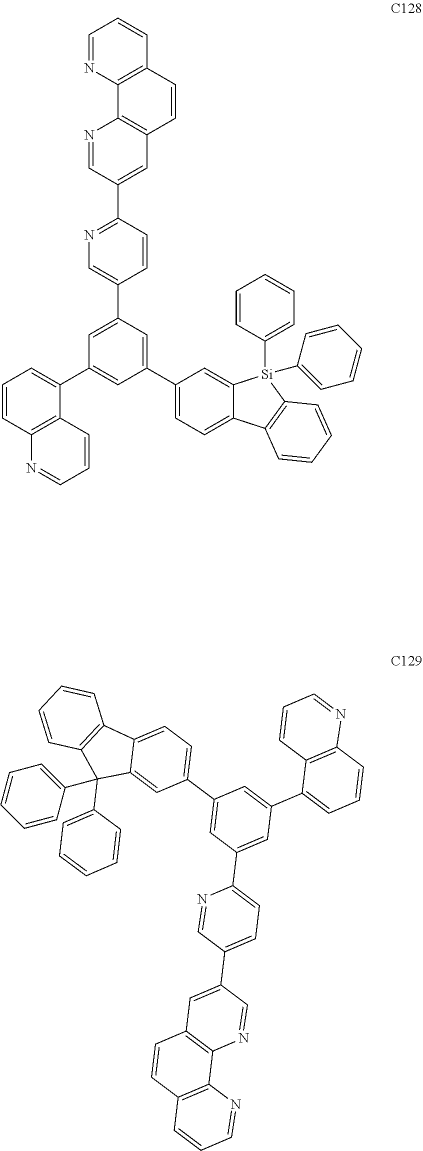 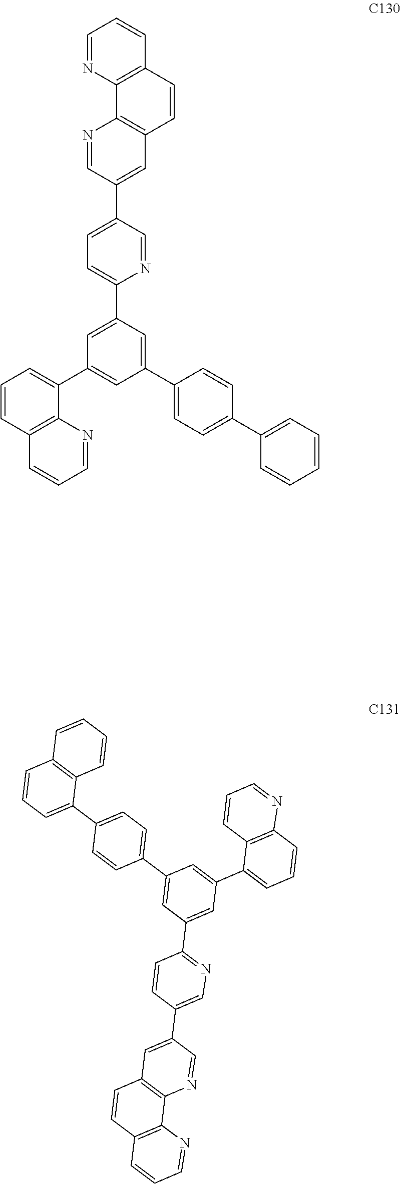 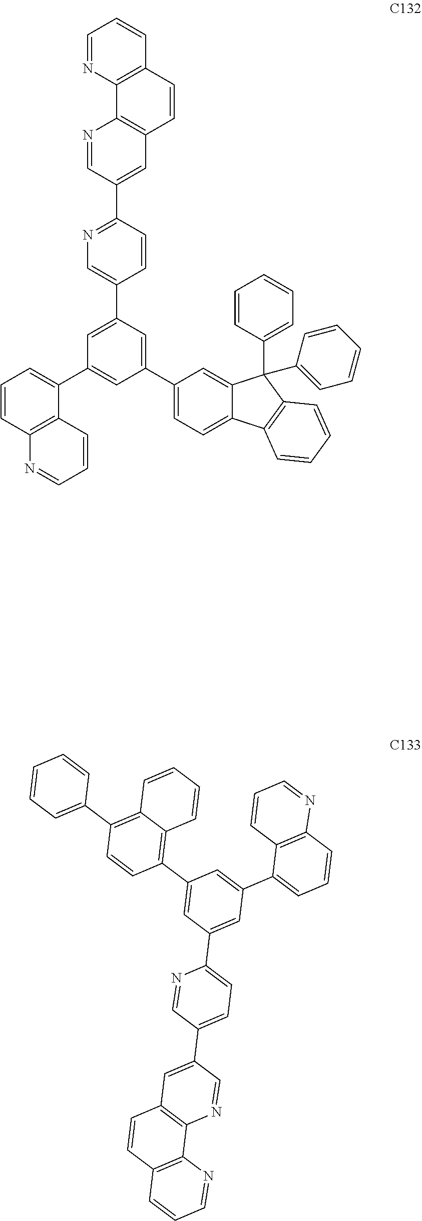 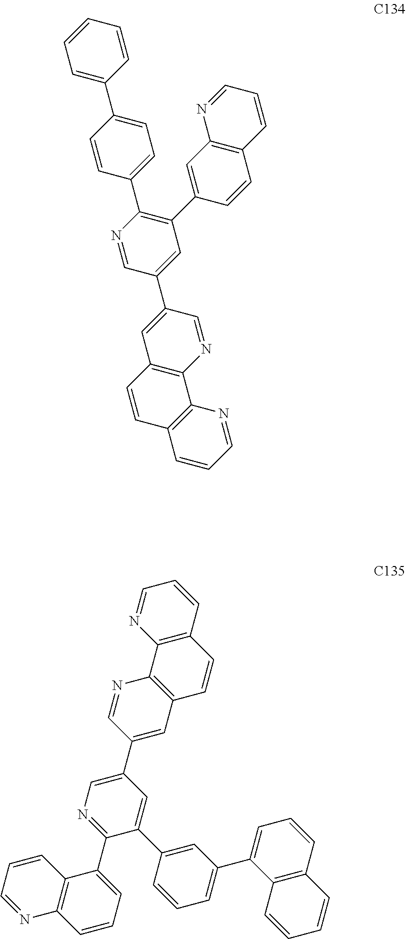 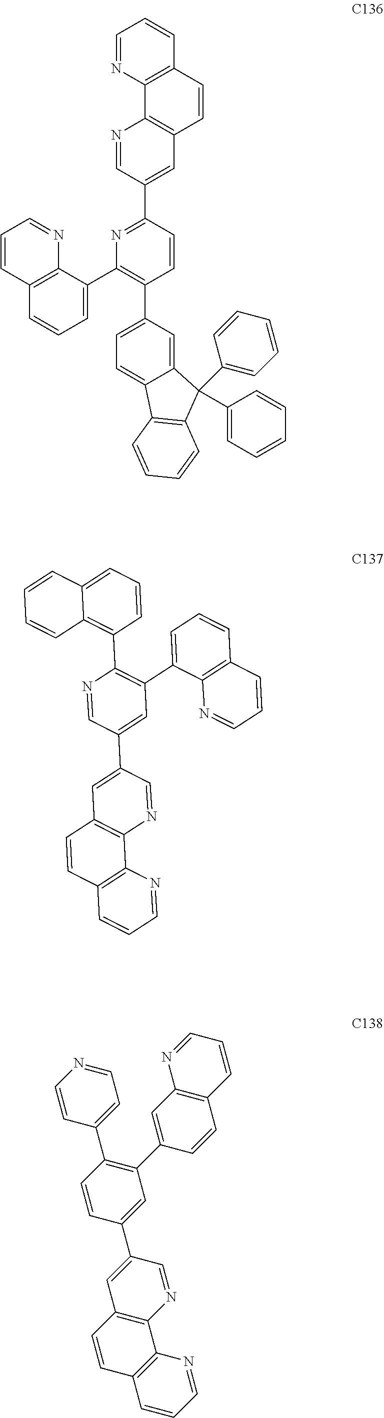 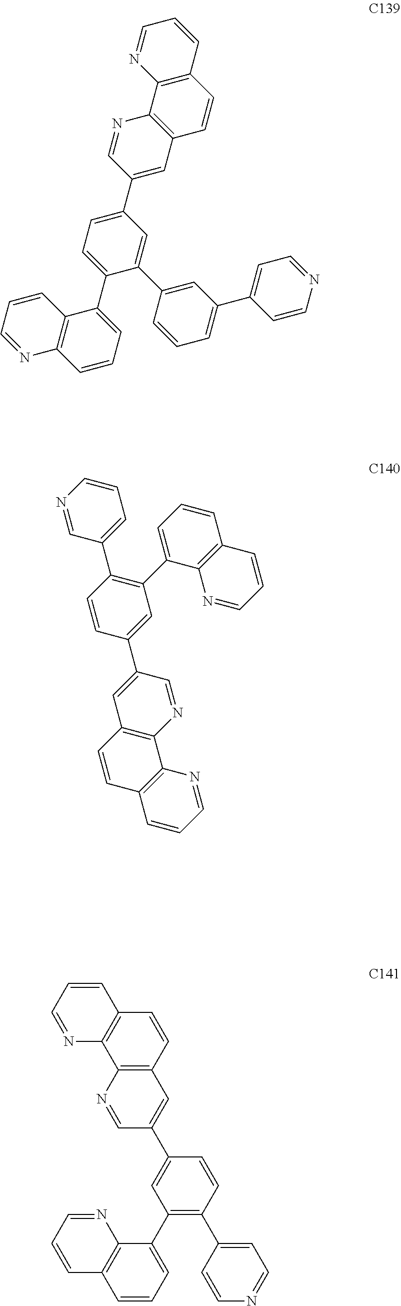 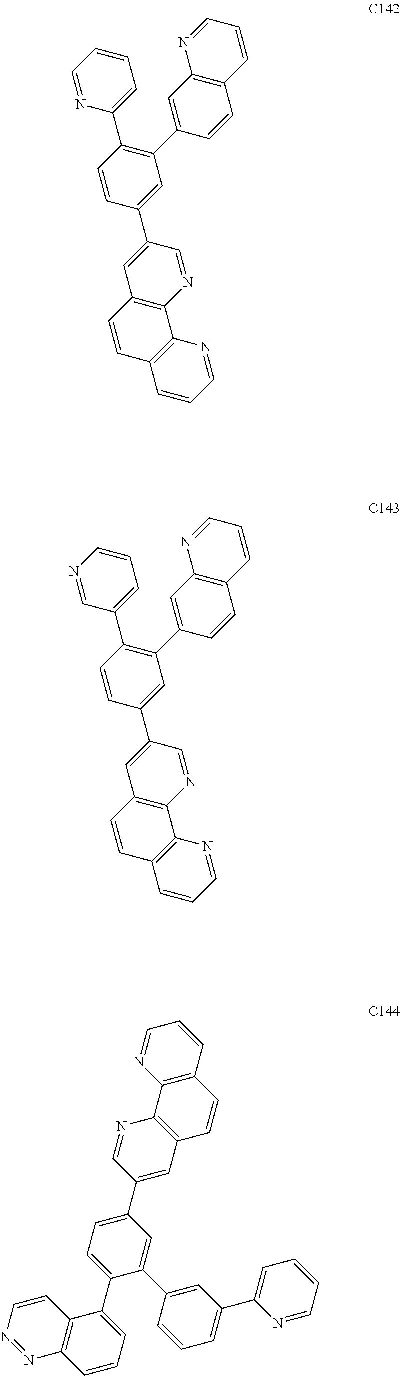 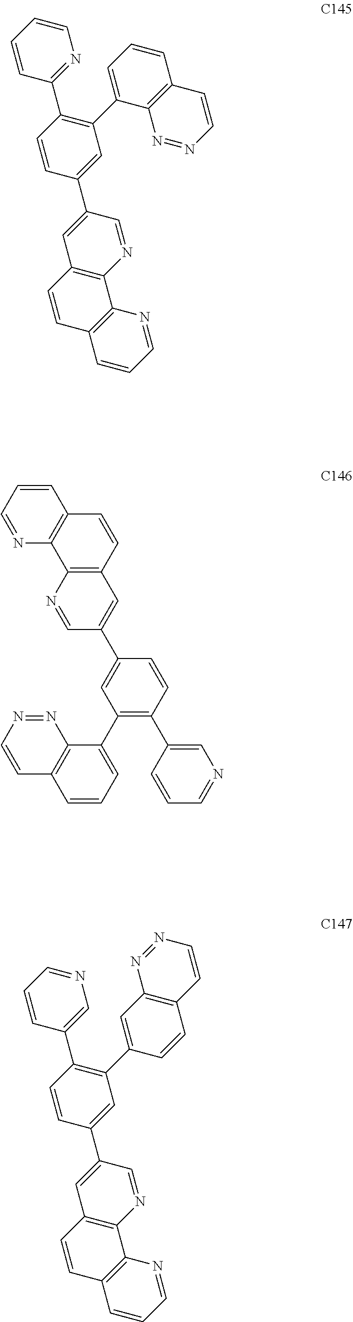 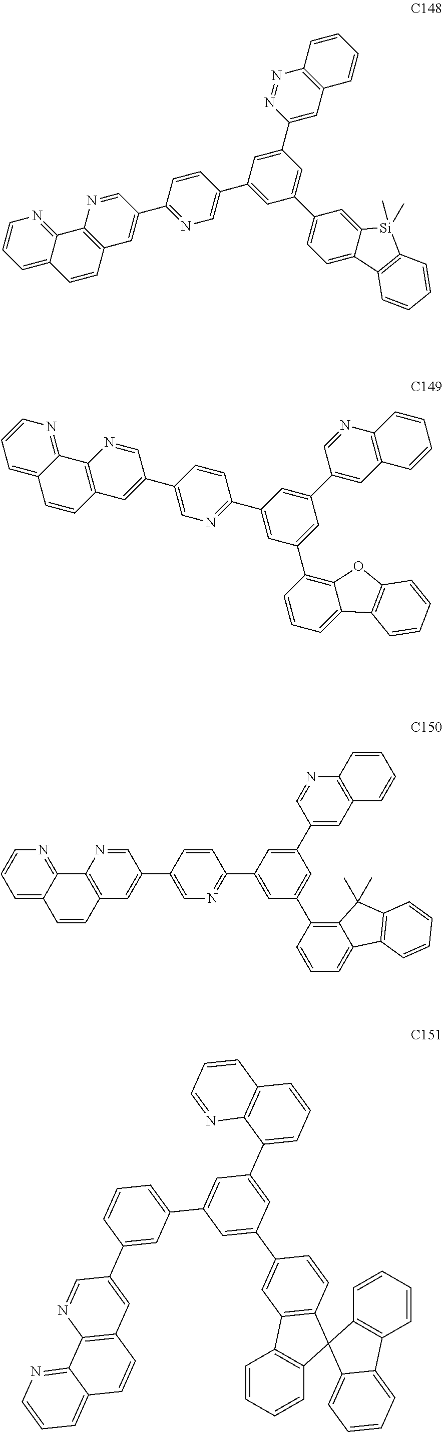 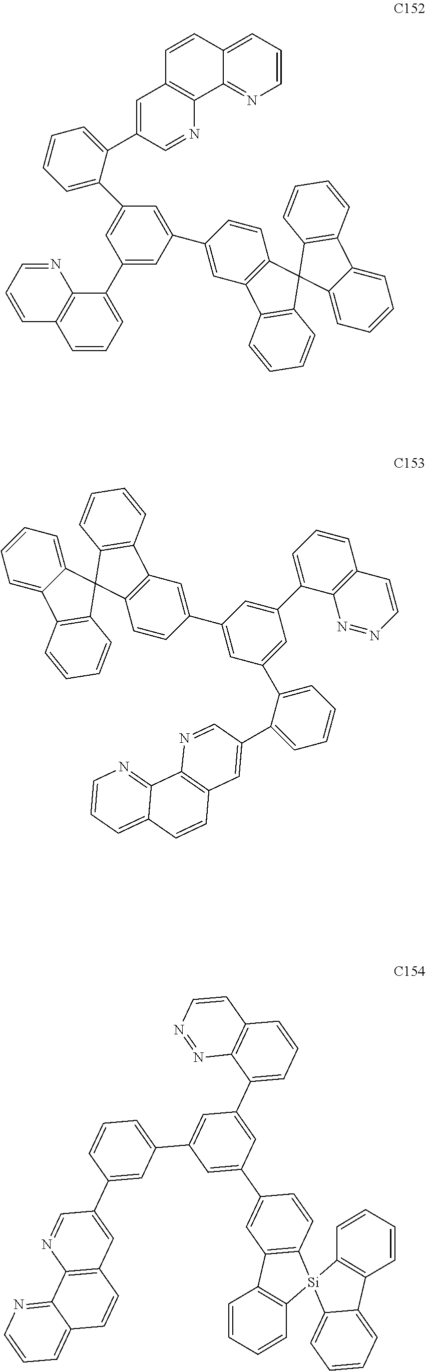 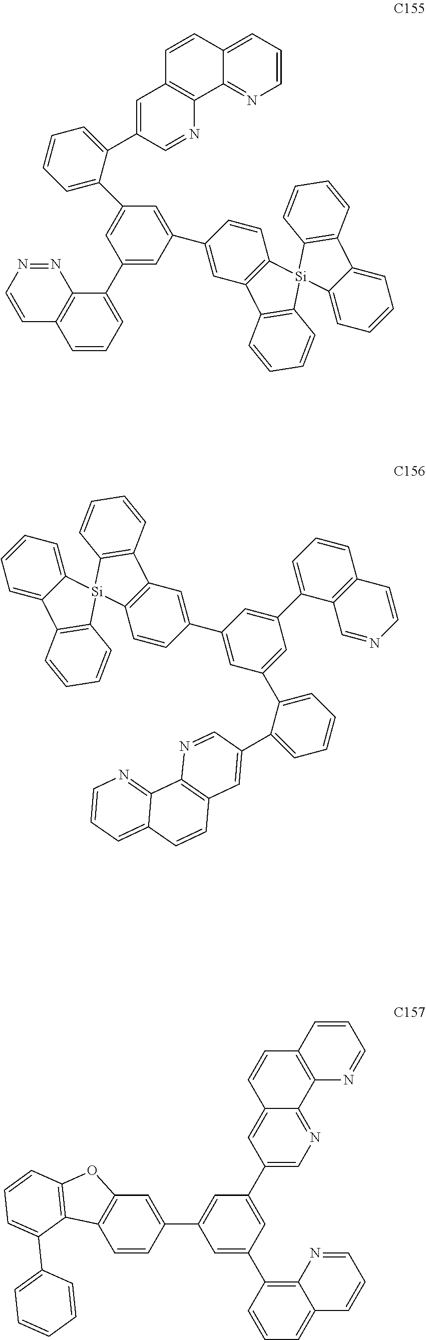 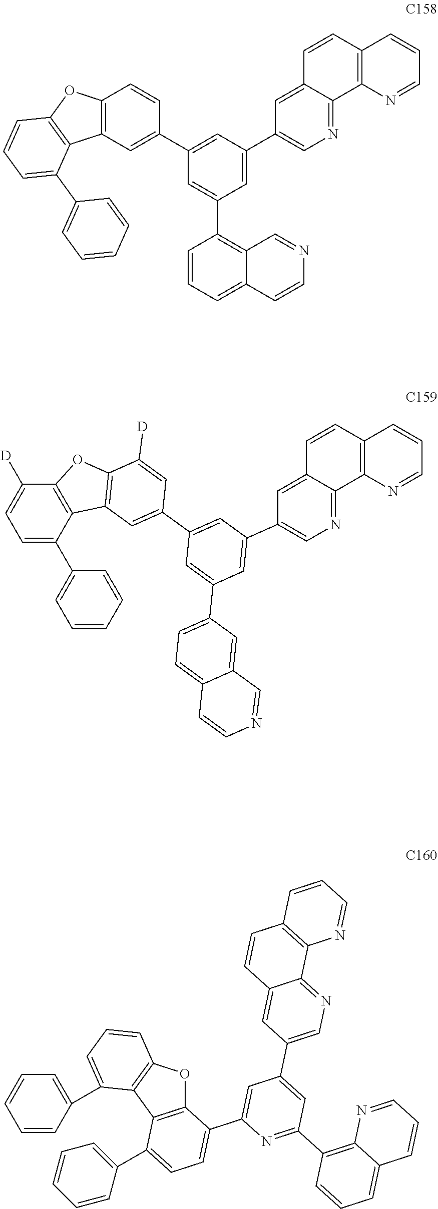 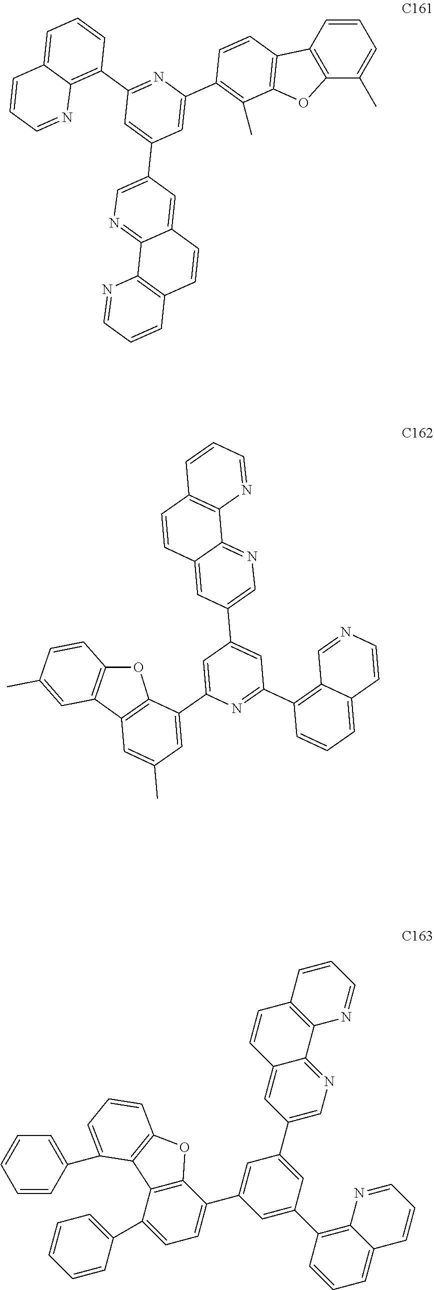 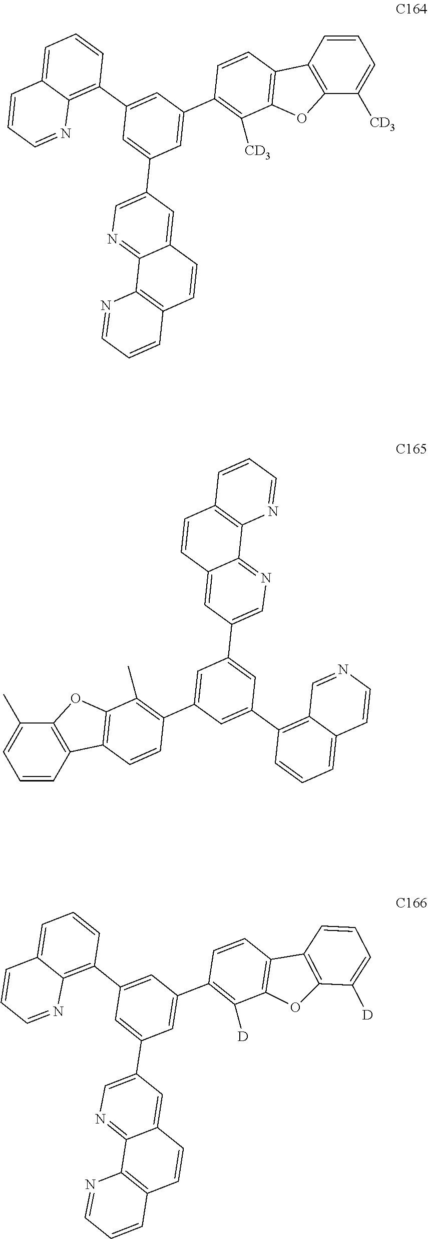 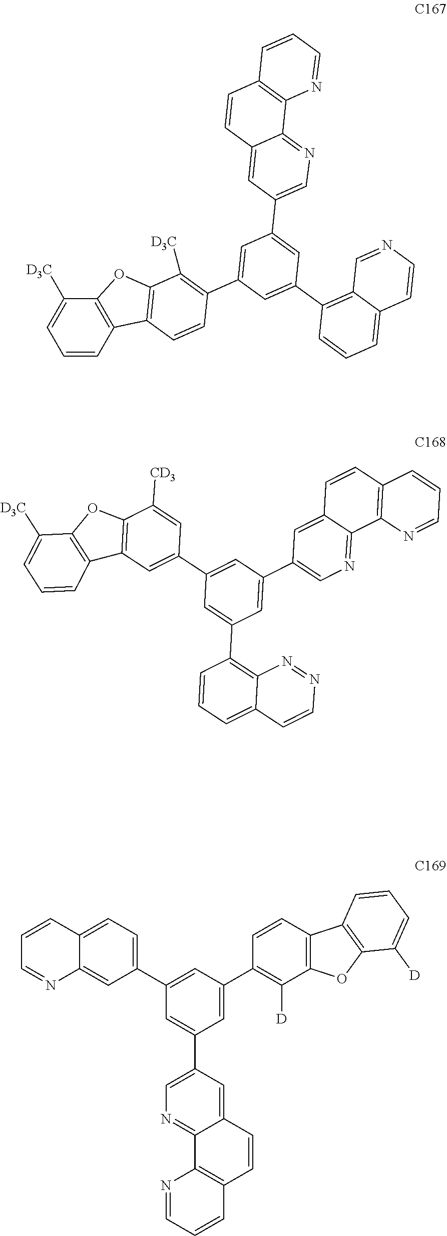 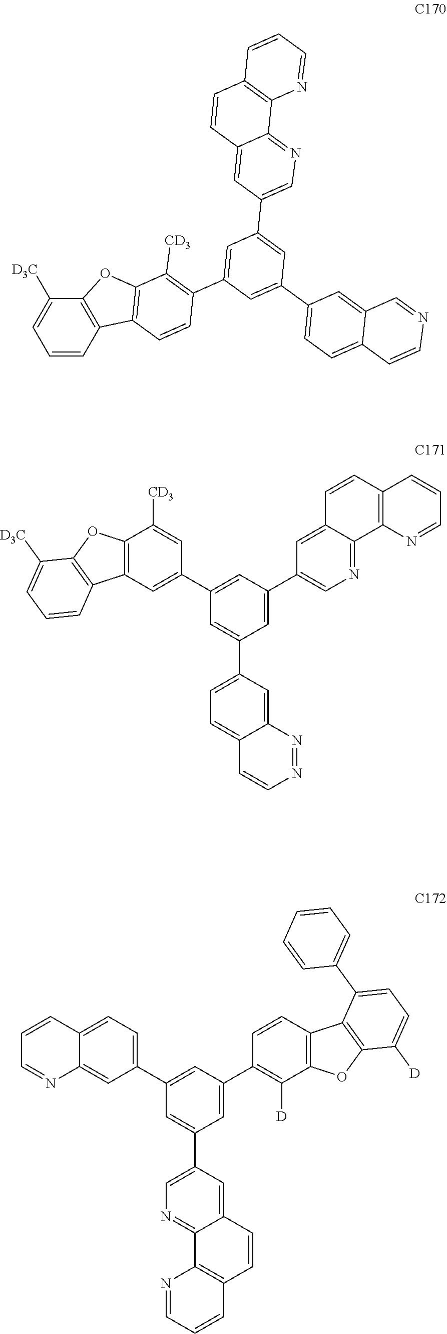 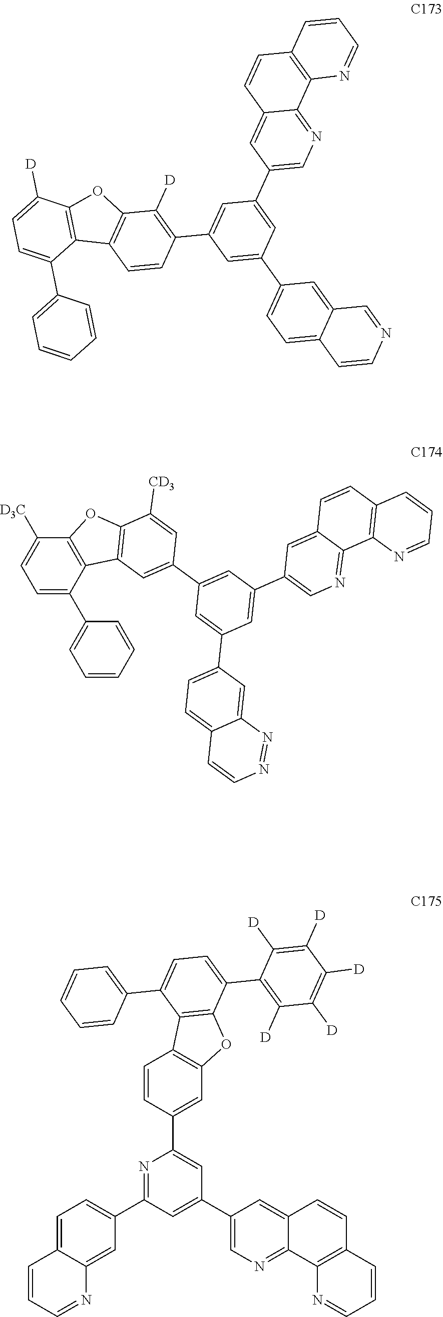 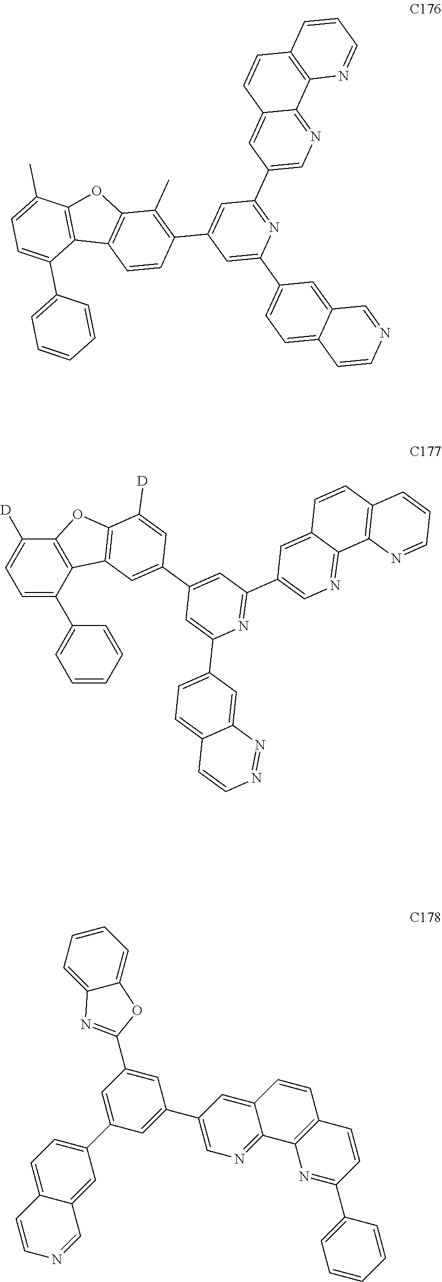 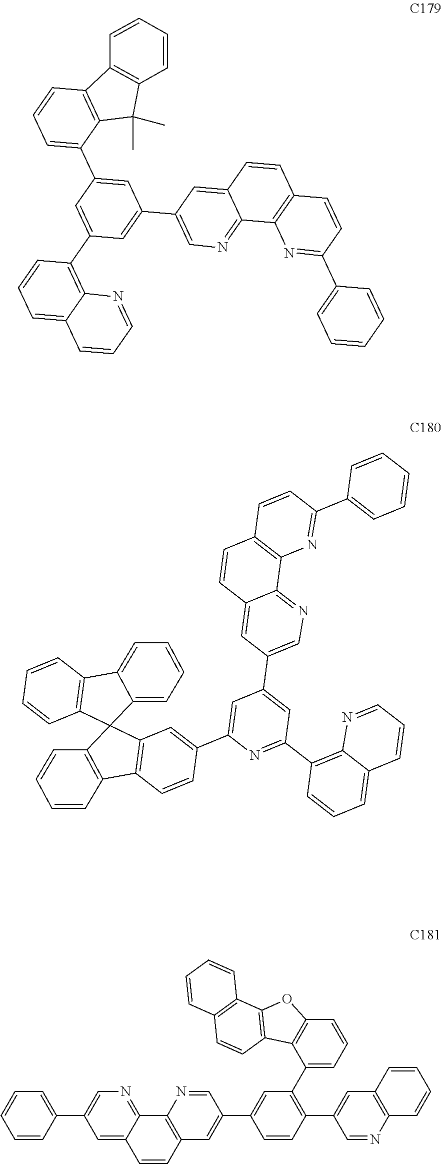 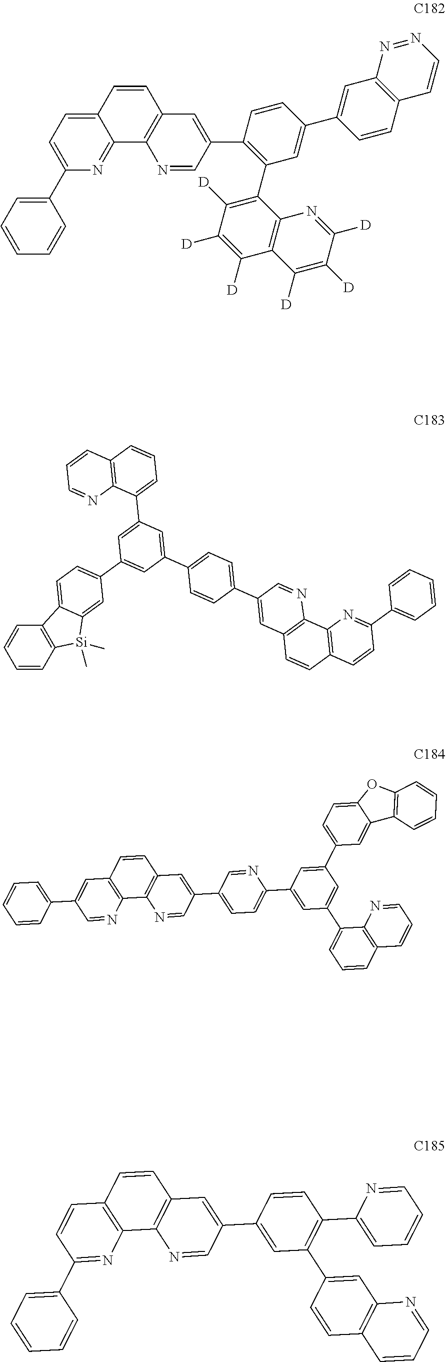 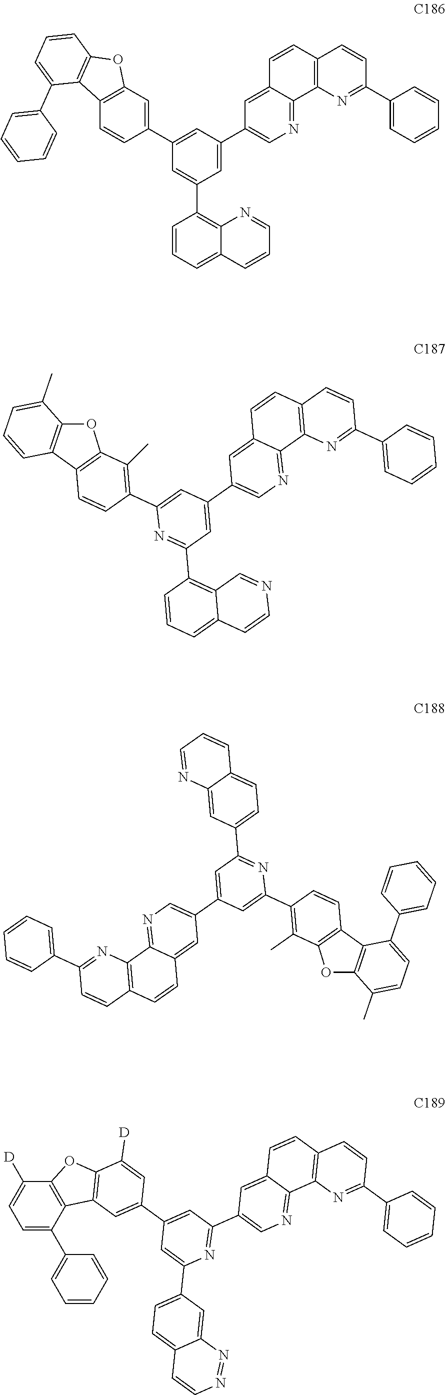 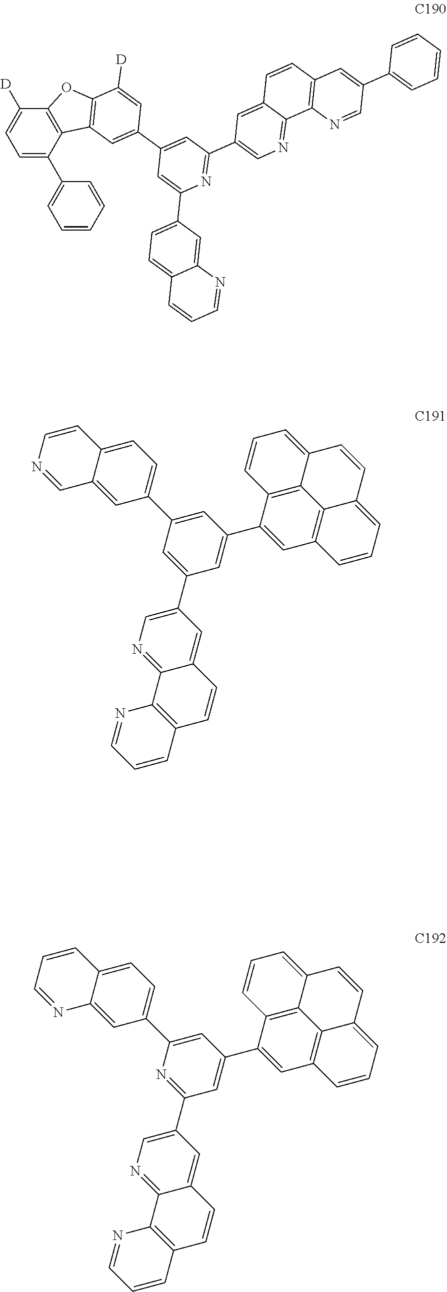 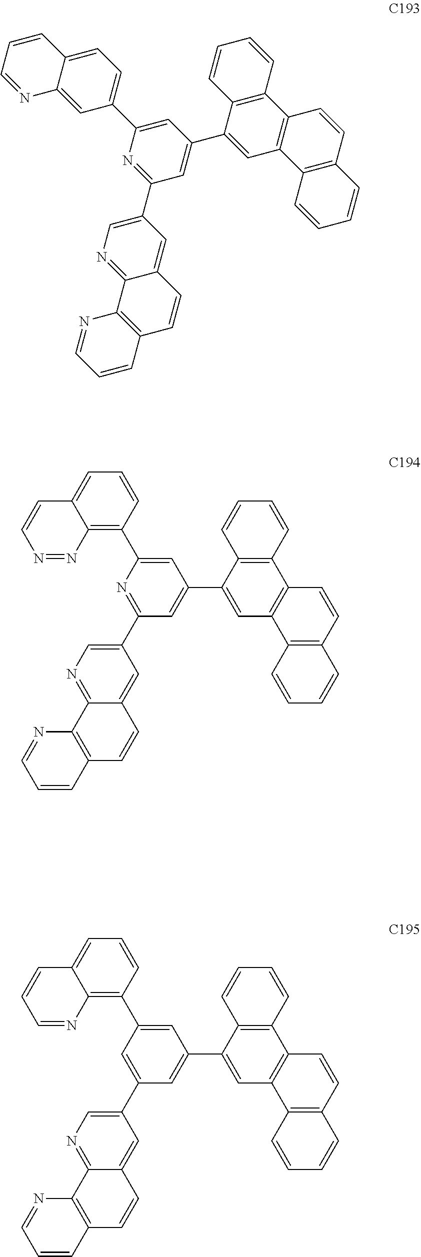 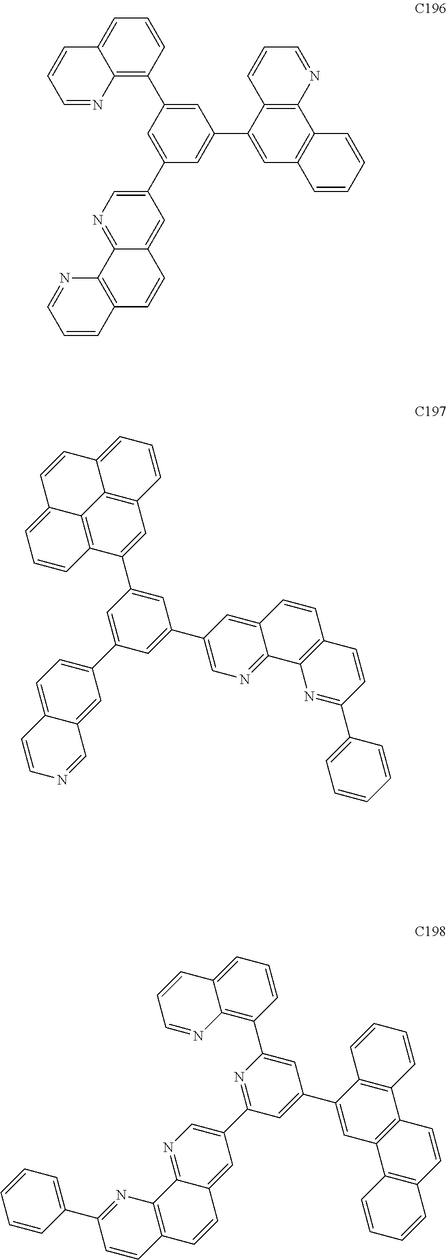 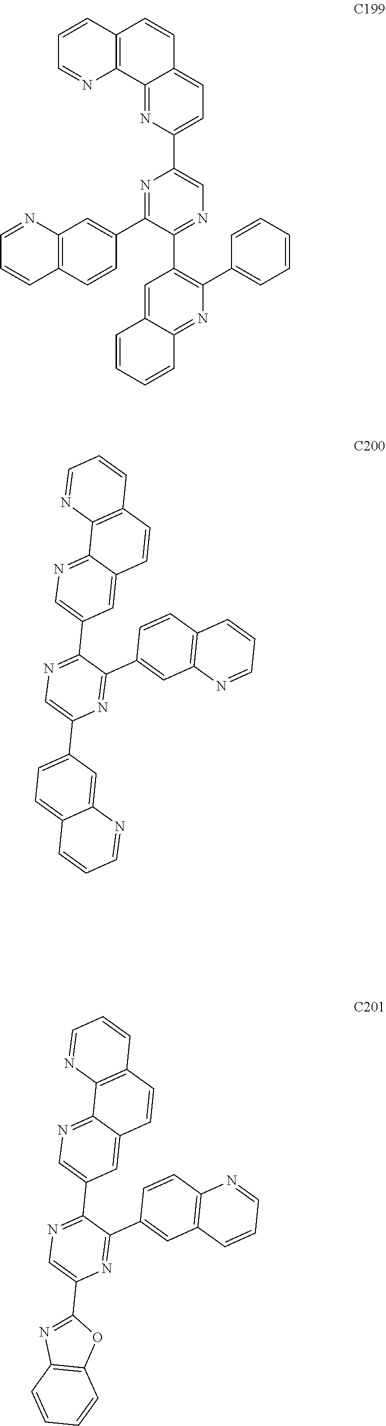 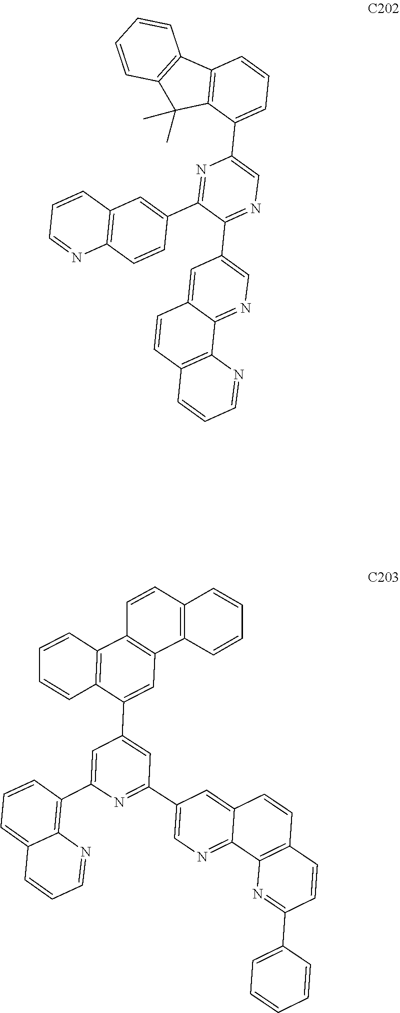 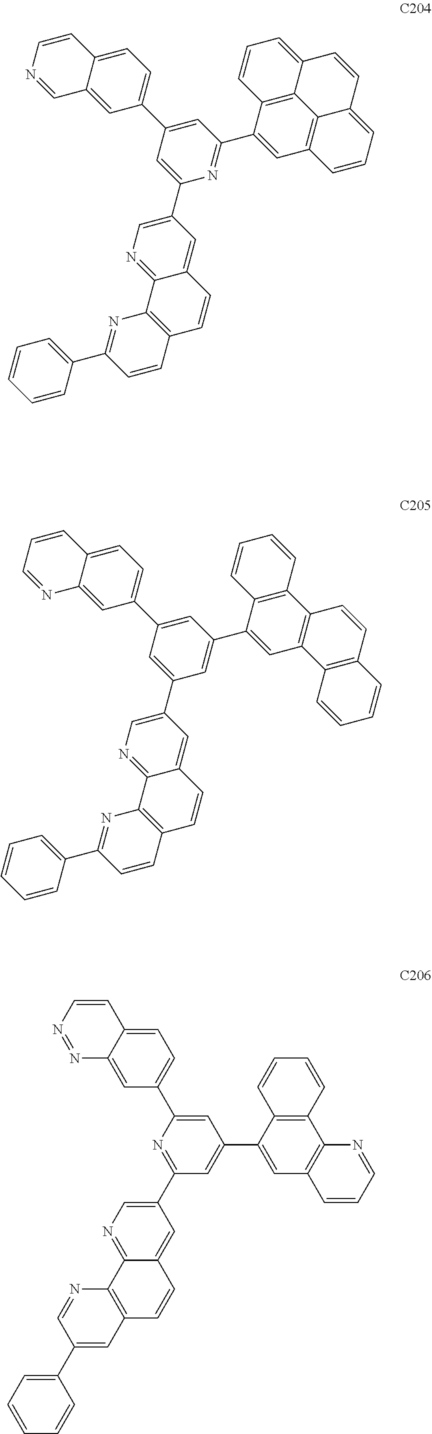 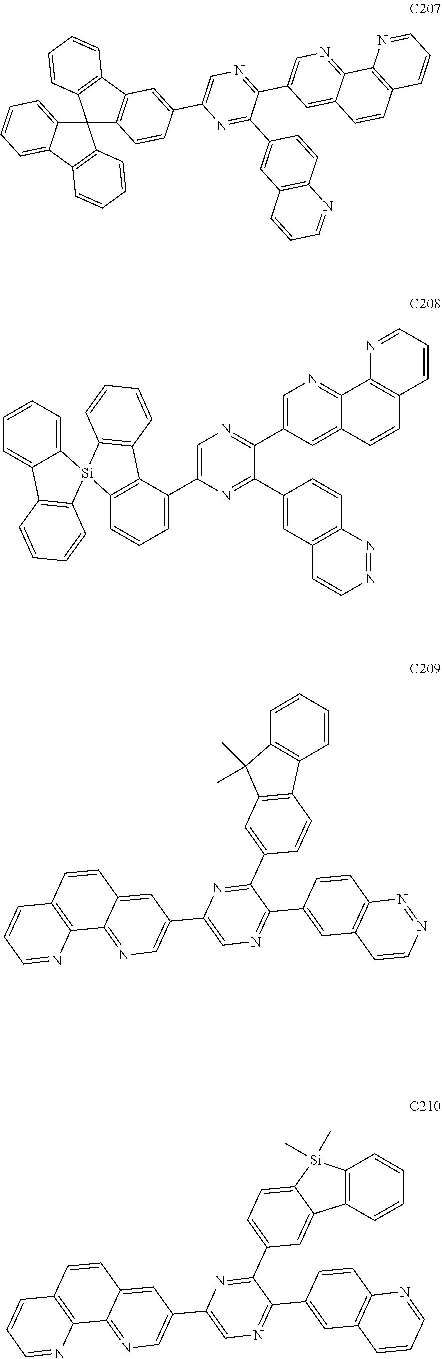 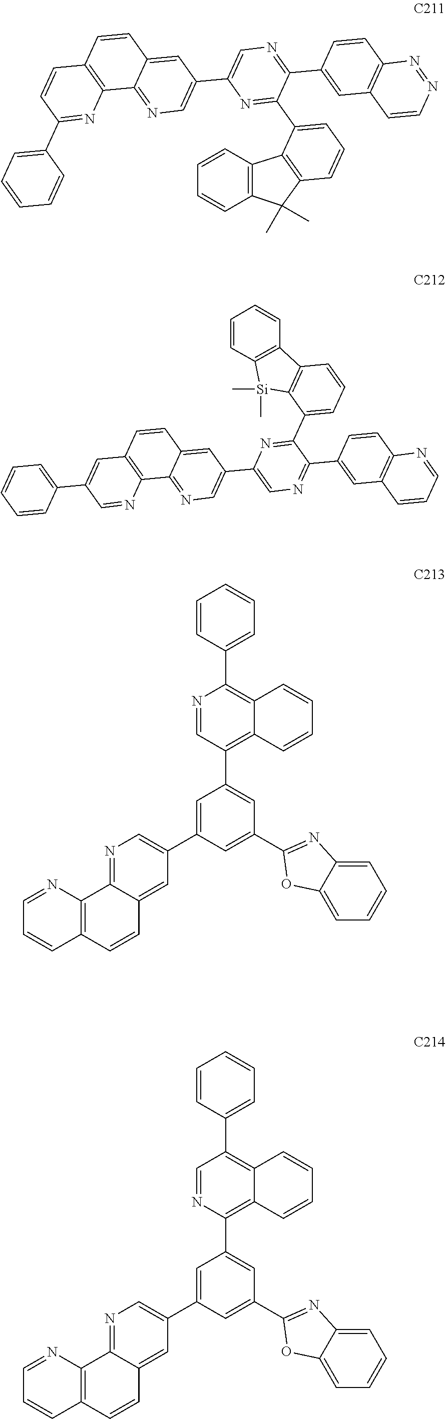 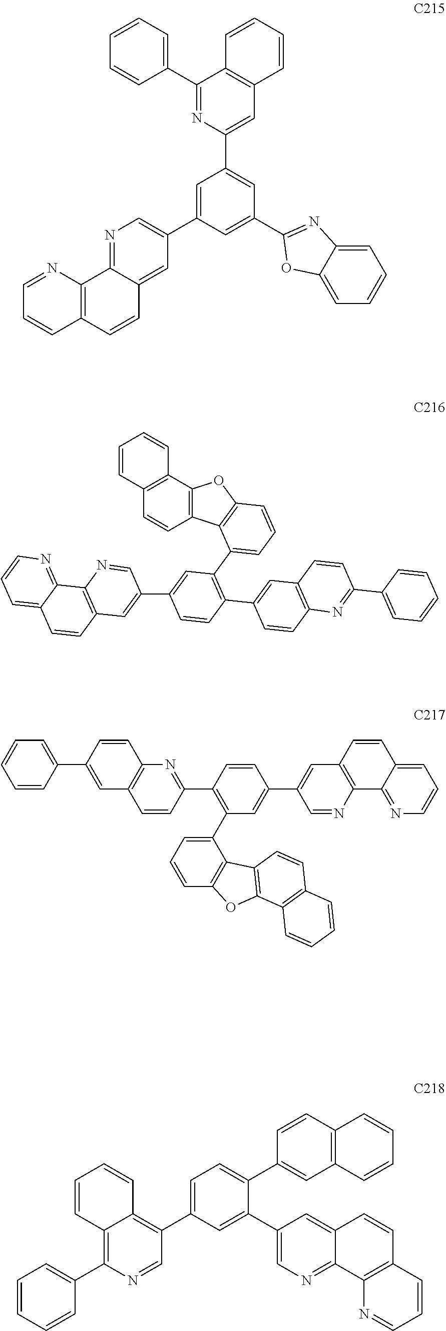 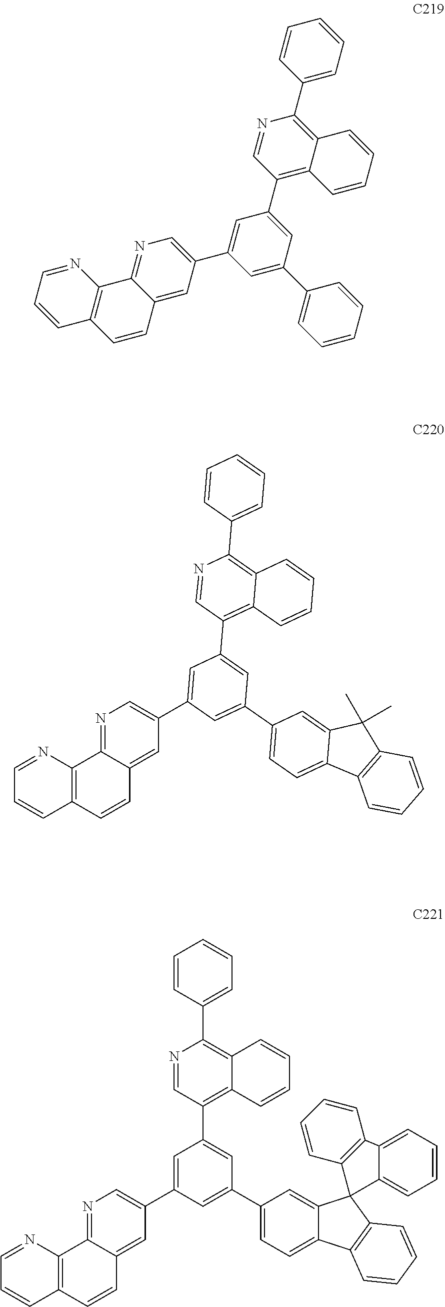 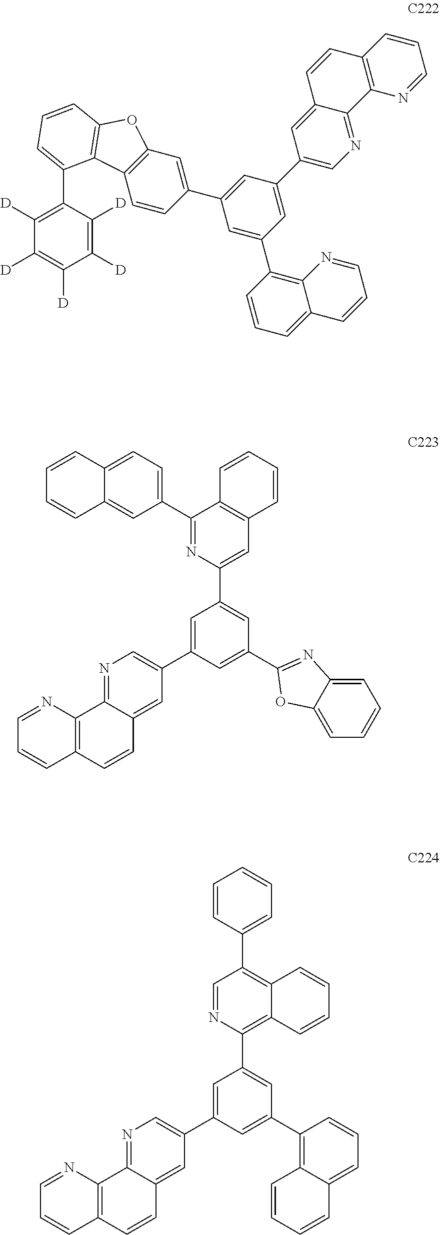 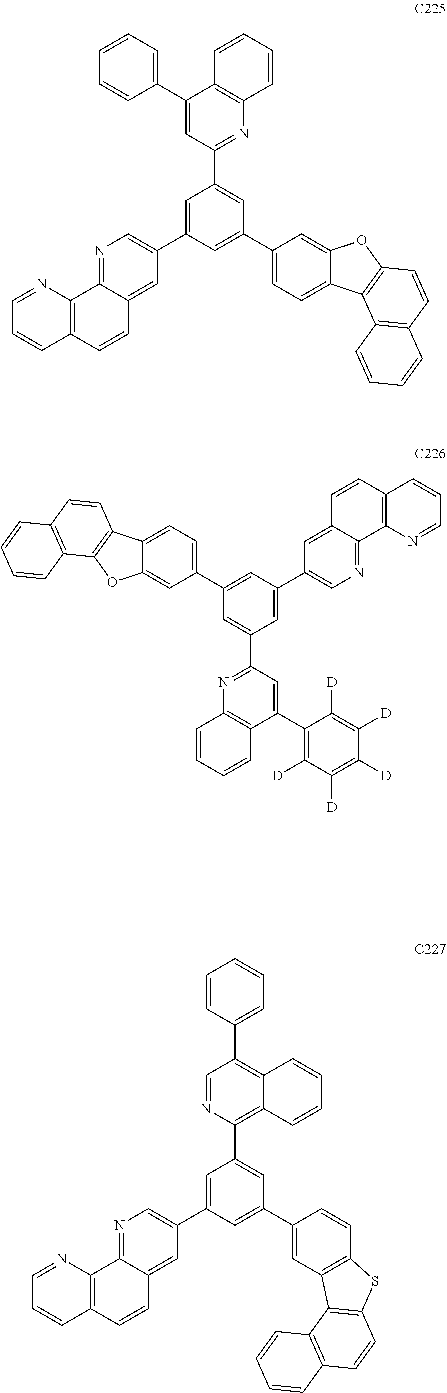 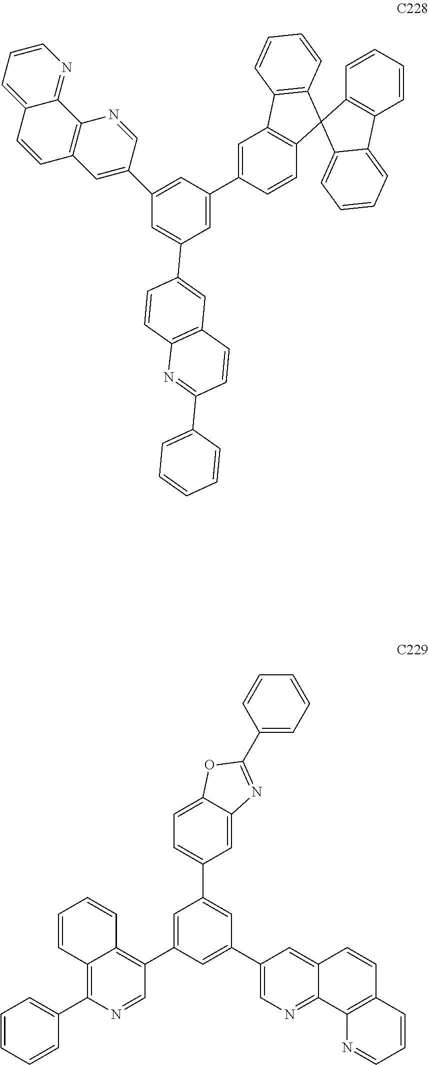 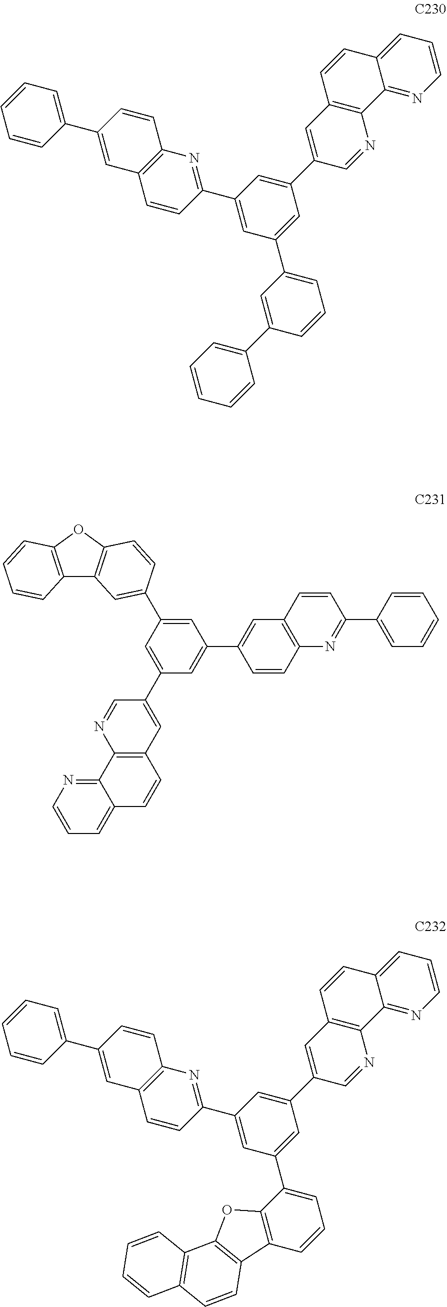 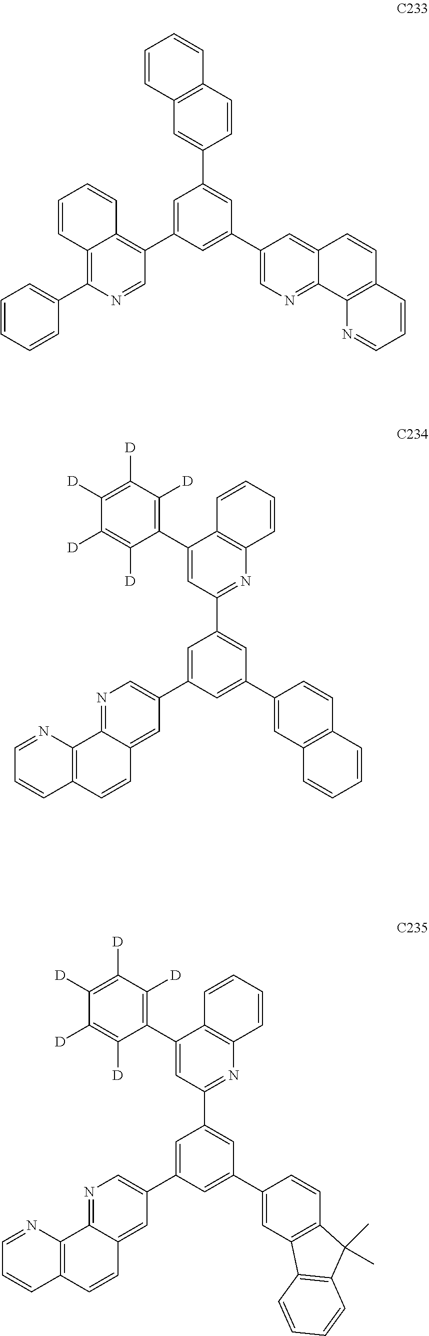 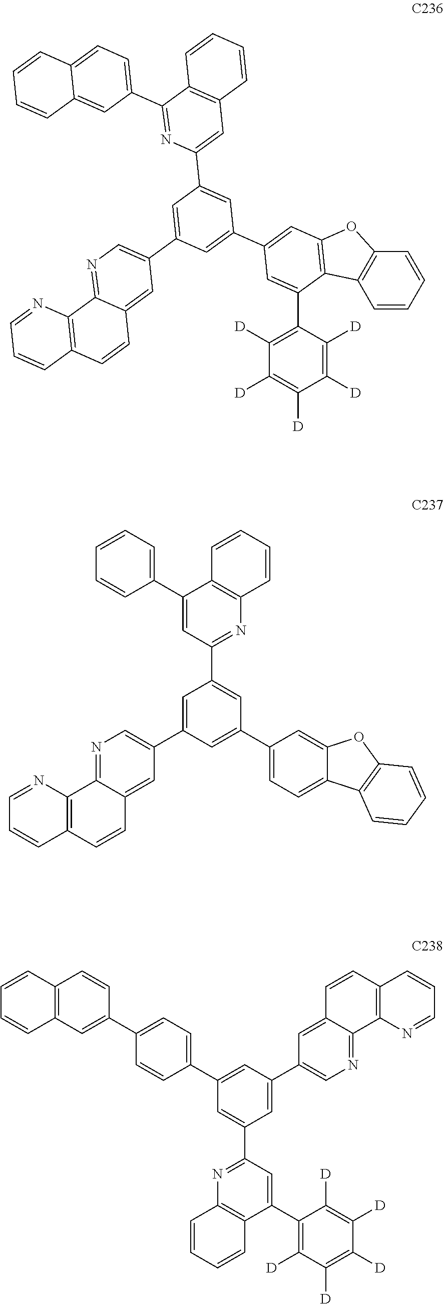 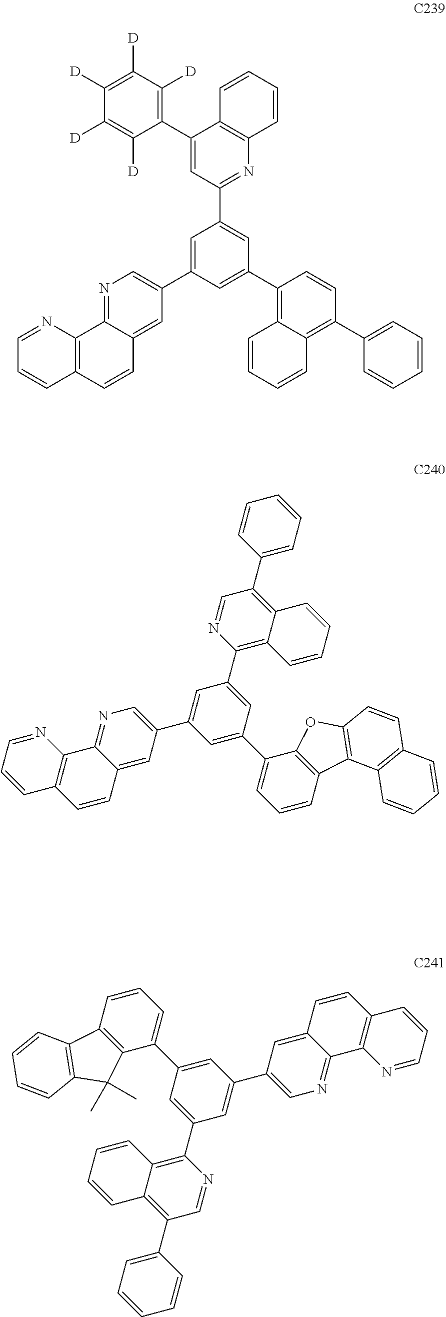 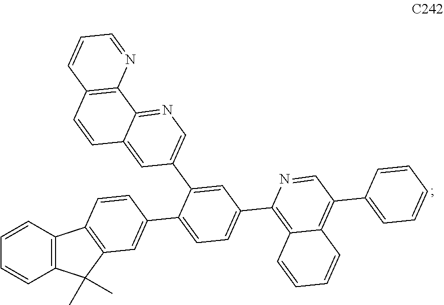
wherein D represents deuterium.

10. An electron transport layer material comprising an organic compound, wherein the organic compound has a structure represented by Formula I: 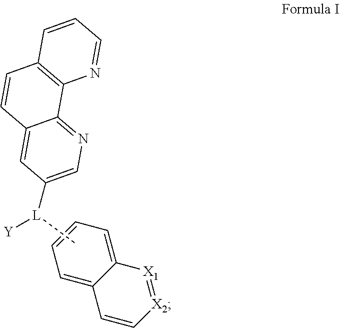
wherein X₁ and X₂ are each independently selected from CR₁ or N; R₁ is selected from hydrogen, a deuterium atom, substituted or unsubstituted C1 to C20 alkyl, substituted or unsubstituted C6 to C30 aryl, substituted or unsubstituted C3 to C20 cycloalkyl, substituted or unsubstituted C1 to C20 alkoxy, substituted or unsubstituted C3 to C30 heterocyclyl or a substituted or unsubstituted C6 to C30 fused ring group;
L is selected from substituted or unsubstituted C6 to C30 aryl or substituted or unsubstituted C3 to C30 heteroaryl; and
Y is selected from substituted or unsubstituted C6 to C30 aryl or substituted or unsubstituted C3 to C30 heteroaryl.

11. An OLED device, comprising an anode, a cathode, at least two organic light-emitting units disposed between the anode and cathode, and a charge generation layer connected to adjacent light-emitting units, wherein the charge generation layer or each of the at least two organic light-emitting units comprises an organic compound, wherein the organic compound has a structure represented by Formula I: 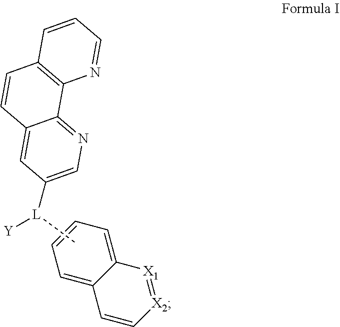
wherein X₁ and X₂ are each independently selected from CR₁ or N; R₁ is selected from hydrogen, a deuterium atom, substituted or unsubstituted C1 to C20 alkyl, substituted or unsubstituted C6 to C30 aryl, substituted or unsubstituted C3 to C20 cycloalkyl, substituted or unsubstituted C1 to C20 alkoxy, substituted or unsubstituted C3 to C30 heterocyclyl or a substituted or unsubstituted C6 to C30 fused ring group;
L is selected from substituted or unsubstituted C6 to C30 aryl or substituted or unsubstituted C3 to C30 heteroaryl; and
Y is selected from substituted or unsubstituted C6 to C30 aryl or substituted or unsubstituted C3 to C30 heteroaryl.

12. The OLED device according to claim 11, wherein each of the at least two organic light-emitting units comprises any one or a combination of at least two of a hole injection layer, a hole transport layer, a light-emitting layer, an electron blocking layer, a hole blocking layer, an electron transport layer and an electron injection layer.

13. The OLED device according to claim 11, wherein each of the at least two organic light-emitting units comprises an electron transport layer, and the electron transport layer comprises the organic compound.
